SEQUENCE LISTING (947 sequences)

SEQ ID NO: 1 - RNA, 22 nt, Homo sapiens
uaacacugucugguaaagaugg

SEQ ID NO: 2 - RNA, 22 nt, Homo sapiens
ugagaacugaauuccauaggcu

SEQ ID NO: 3 - RNA, 23 nt, Homo sapiens
ugugcaaaucuaugcaaaacuga

SEQ ID NO: 4 - RNA, 22 nt, Homo sapiens
uuaaugcuaaucgugauagggg

SEQ ID NO: 5 - RNA, 23 nt, Homo sapiens
uguaguguuuccuacuuuaugga

SEQ ID NO: 6 - RNA, 22 nt, Homo sapiens
uggcucaguucagcaggaacag

SEQ ID NO: 7 - RNA, 20 nt, Homo sapiens
cauaaaguagaaagcacuac

SEQ ID NO: 8 - RNA, 23 nt, Homo sapiens
ugugcaaauccaugcaaaacuga

SEQ ID NO: 9 - RNA, 23 nt, Homo sapiens
uaaggugcaucuagugcagauag

SEQ ID NO: 10 - RNA, 23 nt, Homo sapiens
caaagugcuuacagugcagguag

SEQ ID NO: 11 - RNA, 21 nt, Homo sapiens
ugucaguuugucaaauacccc

SEQ ID NO: 12 - RNA, 20 nt, Homo sapiens
cuuccucgucugucugcccc

SEQ ID NO: 13 - RNA, 23 nt, Homo sapiens
gacacgggcgacagcugcggccc

SEQ ID NO: 14 - RNA, 22 nt, Homo sapiens
ugagguaguagguuguauaguu

SEQ ID NO: 15 - RNA, 22 nt, Homo sapiens
ugagguaguagguugugugguu

SEQ ID NO: 16 - RNA, 22 nt, Homo sapiens
ugagguaguagguuguaugguu

SEQ ID NO: 17 - RNA, 21 nt, Homo sapiens
agagguaguagguugcauagu

SEQ ID NO: 18 - RNA, 21 nt, Homo sapiens
ugagguaggagguuguauagu

SEQ ID NO: 19 - RNA, 22 nt, Homo sapiens
ugagguaguagauuguauaguu

SEQ ID NO: 20 - RNA, 21 nt, Homo sapiens
ugagguaguaguuuguacagu

SEQ ID NO: 21 - RNA, 21 nt, Homo sapiens
ugagguaguaguuugugcugu

SEQ ID NO: 22 - RNA, 21 nt, Homo sapiens
uggaauguaaagaaguaugua

SEQ ID NO: 23 - RNA, 22 nt, Homo sapiens
uggaagacuagugauuuuguug

SEQ ID NO: 24 - RNA, 23 nt, Homo sapiens
uacccuguagauccgaauuugug

SEQ ID NO: 25 - RNA, 22 nt, Homo sapiens
uacccuguagaaccgaauuugu

SEQ ID NO: 26 - RNA, 22 nt, Homo sapiens
uagcagcacauaaugguuugug

SEQ ID NO: 27 - RNA, 22 nt, Homo sapiens
uagcagcacaucaugguuuaca

SEQ ID NO: 28 - RNA, 22 nt, Homo sapiens
uagcagcacguaaauauuggcg

SEQ ID NO: 29 - RNA, 20 nt, Homo sapiens
acugcagugaaggcacuugu

SEQ ID NO: 30 - RNA, 24 nt, Homo sapiens
caaagugcuuacagugcagguagu

SEQ ID NO: 31 - RNA, 22 nt, Homo sapiens
uaaggugcaucuagugcagaua

SEQ ID NO: 32 - RNA, 22 nt, Homo sapiens
uagcuuaucagacugauguuga

SEQ ID NO: 33 - RNA, 22 nt, Homo sapiens
aagcugccaguugaagaacugu

SEQ ID NO: 34 - RNA, 21 nt, Homo sapiens
aucacauugccagggauuucc

SEQ ID NO: 35 - RNA, 21 nt, Homo sapiens
aucacauugccagggauuacc

SEQ ID NO: 36 - RNA, 22 nt, Homo sapiens
cauugcacuugucucggucuga

SEQ ID NO: 37 - RNA, 21 nt, Homo sapiens
uucaaguaauccaggauaggc

SEQ ID NO: 38 - RNA, 22 nt, Homo sapiens
uucaaguaauccaggauaggcu

SEQ ID NO: 39 - RNA, 22 nt, Homo sapiens
uucaaguaauucaggauagguu

SEQ ID NO: 40 - RNA, 21 nt, Homo sapiens
uucaaguaauucaggauaggu

SEQ ID NO: 41 - RNA, 21 nt, Homo sapiens
uucacaguggcuaaguuccgc

SEQ ID NO: 42 - RNA, 21 nt, Homo sapiens
uucacaguggcuaaguucugc

SEQ ID NO: 43 - RNA, 22 nt, Homo sapiens
aaggagcucacagucuauugag

SEQ ID NO: 44 - RNA, 21 nt, Homo sapiens
uagcaccaucugaaaucgguu

SEQ ID NO: 45 - RNA, 23 nt, Homo sapiens
uagcaccauuugaaaucaguguu

SEQ ID NO: 46 - RNA, 20 nt, Homo sapiens
uagcaccauuugaaaucggu

SEQ ID NO: 47 - RNA, 22 nt, Homo sapiens
cuuucagucggauguuugcagc

SEQ ID NO: 48 - RNA, 22 nt, Homo sapiens
uguaaacauccucgacuggaag

SEQ ID NO: 49 - RNA, 23 nt, Homo sapiens
uguaaacauccuacacucucagc

SEQ ID NO: 50 - RNA, 22 nt, Homo sapiens
uguaaacauccccgacuggaag

SEQ ID NO: 51 - RNA, 20 nt, Homo sapiens
uguaaacauccuugacugga

SEQ ID NO: 52 - RNA, 22 nt, Homo sapiens
cuuucagucggauguuuacagc

SEQ ID NO: 53 - RNA, 21 nt, Homo sapiens
uauugcacauuacuaaguugc

SEQ ID NO: 54 - RNA, 19 nt, Homo sapiens
gugcauuguaguugcauug

SEQ ID NO: 55 - RNA, 23 nt, Homo sapiens
uggcagugucuuagcugguuguu

SEQ ID NO: 56 - RNA, 22 nt, Homo sapiens
uggcagugucuuagcugguugu

SEQ ID NO: 57 - RNA, 23 nt, Homo sapiens
uaggcagugucauuagcugauug

SEQ ID NO: 58 - RNA, 23 nt, Homo sapiens
aggcaguguaguuagcugauugc

SEQ ID NO: 59 - RNA, 21 nt, Homo sapiens
uauugcacuugucccggccug

SEQ ID NO: 60 - RNA, 22 nt, Homo sapiens
uauugcacuugucccggccugu

SEQ ID NO: 61 - RNA, 22 nt, Homo sapiens
aaagugcuguucgugcagguag

SEQ ID NO: 62 - RNA, 22 nt, Homo sapiens
uucaacggguauuuauugagca

SEQ ID NO: 63 - RNA, 22 nt, Homo sapiens
uuuggcacuagcacauuuuugc

SEQ ID NO: 64 - RNA, 22 nt, Homo sapiens
aacccguagauccgaucuugug

SEQ ID NO: 65 - RNA, 22 nt, Homo sapiens
cacccguagaaccgaccuugcg

SEQ ID NO: 66 - RNA, 22 nt, Homo sapiens
aacccguagauccgaacuugug

SEQ ID NO: 67 - RNA, 22 nt, Homo sapiens
uacaguacugugauaacugaag

SEQ ID NO: 68 - RNA, 23 nt, Homo sapiens
agcagcauuguacagggcuauga

SEQ ID NO: 69 - RNA, 20 nt, Homo sapiens
ucaaaugcucagacuccugu

SEQ ID NO: 70 - RNA, 21 nt, Homo sapiens
uaaagugcugacagugcagau

SEQ ID NO: 71 - RNA, 23 nt, Homo sapiens
agcagcauuguacagggcuauca

SEQ ID NO: 72 - RNA, 23 nt, Homo sapiens
uggagugugacaaugguguuugu

SEQ ID NO: 73 - RNA, 22 nt, Homo sapiens
uuaaggcacgcggugaaugcca

SEQ ID NO: 74 - RNA, 23 nt, Homo sapiens
ucccugagacccuuuaaccugug

SEQ ID NO: 75 - RNA, 22 nt, Homo sapiens
ucccugagacccuaacuuguga

SEQ ID NO: 76 - RNA, 21 nt, Homo sapiens
ucguaccgugaguaauaaugc

SEQ ID NO: 77 - RNA, 21 nt, Homo sapiens
cauuauuacuuuugguacgcg

SEQ ID NO: 78 - RNA, 22 nt, Homo sapiens
ucggauccgucugagcuuggcu

SEQ ID NO: 79 - RNA, 22 nt, Homo sapiens
ucacagugaaccggucucuuuu

SEQ ID NO: 80 - RNA, 22 nt, Homo sapiens
cagugcaauguuaaaagggcau

SEQ ID NO: 81 - RNA, 22 nt, Homo sapiens
cagugcaaugaugaaagggcau

SEQ ID NO: 82 - RNA, 22 nt, Homo sapiens
uaacagucuacagccauggucg

SEQ ID NO: 83 - RNA, 22 nt, Homo sapiens
uugguccccuucaaccagcugu

SEQ ID NO: 84 - RNA, 21 nt, Homo sapiens
ugugacugguugaccagaggg

SEQ ID NO: 85 - RNA, 23 nt, Homo sapiens
uauggcuuuuuauuccuauguga

SEQ ID NO: 86 - RNA, 22 nt, Homo sapiens
uauggcuuuucauuccuaugug

SEQ ID NO: 87 - RNA, 21 nt, Homo sapiens
agugguuuuacccuaugguag

SEQ ID NO: 88 - RNA, 22 nt, Homo sapiens
ugagaugaagcacuguagcuca

SEQ ID NO: 89 - RNA, 24 nt, Homo sapiens
guccaguuuucccaggaaucccuu

SEQ ID NO: 90 - RNA, 22 nt, Homo sapiens
ugagaacugaauuccauggguu

SEQ ID NO: 91 - RNA, 20 nt, Homo sapiens
guguguggaaaugcuucugc

SEQ ID NO: 92 - RNA, 22 nt, Homo sapiens
ucagugcacuacagaacuuugu

SEQ ID NO: 93 - RNA, 22 nt, Homo sapiens
ucagugcaucacagaacuuugu

SEQ ID NO: 94 - RNA, 22 nt, Homo sapiens
ucuggcuccgugucuucacucc

SEQ ID NO: 95 - RNA, 22 nt, Homo sapiens
ucucccaacccuuguaccagug

SEQ ID NO: 96 - RNA, 22 nt, Homo sapiens
ucagugcaugacagaacuuggg

SEQ ID NO: 97 - RNA, 21 nt, Homo sapiens
ucagugcaugacagaacuugg

SEQ ID NO: 98 - RNA, 20 nt, Homo sapiens
uugcauagucacaaaaguga

SEQ ID NO: 99 - RNA, 20 nt, Homo sapiens
ucacaccugccucgcccccc

SEQ ID NO: 100 - RNA, 22 nt, Homo sapiens
uagguuauccguguugccuucg

SEQ ID NO: 101 - RNA, 22 nt, Homo sapiens
aaucauacacgguugaccuauu

SEQ ID NO: 102 - RNA, 21 nt, Homo sapiens
agugaaugauggguucugacc

SEQ ID NO: 103 - RNA, 23 nt, Homo sapiens
aacauucaacgcugucggugagu

SEQ ID NO: 104 - RNA, 22 nt, Homo sapiens
aacauucaaccugucggugagu

SEQ ID NO: 105 - RNA, 21 nt, Homo sapiens
ugguucuagacuugccaacua

SEQ ID NO: 106 - RNA, 23 nt, Homo sapiens
uauggcacugguagaauucacug

SEQ ID NO: 107 - RNA, 22 nt, Homo sapiens
uggacggagaacugauaagggu

SEQ ID NO: 108 - RNA, 23 nt, Homo sapiens
caaagaauucuccuuuugggcuu

SEQ ID NO: 109 - RNA, 21 nt, Homo sapiens
ucgugucuuguguugcagccg

SEQ ID NO: 110 - RNA, 23 nt, Homo sapiens
gugccuacugagcugauaucagu

SEQ ID NO: 111 - RNA, 22 nt, Homo sapiens
ugauauguuugauauauuaggu

SEQ ID NO: 112 - RNA, 22 nt, Homo sapiens
caacggaaucccaaaagcagcu

SEQ ID NO: 113 - RNA, 21 nt, Homo sapiens
cugaccuaugaauugacagcc

SEQ ID NO: 114 - RNA, 21 nt, Homo sapiens
aacuggccuacaaagucccag

SEQ ID NO: 115 - RNA, 22 nt, Homo sapiens
uguaacagcaacuccaugugga

SEQ ID NO: 116 - RNA, 21 nt, Homo sapiens
uagcagcacagaaauauuggc

SEQ ID NO: 117 - RNA, 21 nt, Homo sapiens
uagguaguuucauguuguugg

SEQ ID NO: 118 - RNA, 21 nt, Homo sapiens
uagguaguuuccuguuguugg

SEQ ID NO: 119 - RNA, 22 nt, Homo sapiens
uucaccaccuucuccacccagc

SEQ ID NO: 120 - RNA, 23 nt, Homo sapiens
cccaguguucagacuaccuguuc

SEQ ID NO: 121 - RNA, 22 nt, Homo sapiens
uacaguagucugcacauugguu

SEQ ID NO: 122 - RNA, 23 nt, Homo sapiens
cccaguguuuagacuaucuguuc

SEQ ID NO: 123 - RNA, 22 nt, Homo sapiens
uaacacugucugguaacgaugu

SEQ ID NO: 124 - RNA, 22 nt, Homo sapiens
uaauacugccggguaaugaugg

SEQ ID NO: 125 - RNA, 22 nt, Homo sapiens
gugaaauguuuaggaccacuag

SEQ ID NO: 126 - RNA, 22 nt, Homo sapiens
uucccuuugucauccuaugccu

SEQ ID NO: 127 - RNA, 22 nt, Homo sapiens
uccuucauuccaccggagucug

SEQ ID NO: 128 - RNA, 22 nt, Homo sapiens
uggaauguaaggaagugugugg

SEQ ID NO: 129 - RNA, 22 nt, Homo sapiens
auaagacgagcaaaaagcuugu

SEQ ID NO: 130 - RNA, 22 nt, Homo sapiens
cugugcgugugacagcggcuga

SEQ ID NO: 131 - RNA, 22 nt, Homo sapiens
uucccuuugucauccuucgccu

SEQ ID NO: 132 - RNA, 21 nt, Homo sapiens
uaacagucuccagucacggcc

SEQ ID NO: 133 - RNA, 22 nt, Homo sapiens
accaucgaccguugauuguacc

SEQ ID NO: 134 - RNA, 21 nt, Homo sapiens
acagcaggcacagacaggcag

SEQ ID NO: 135 - RNA, 21 nt, Homo sapiens
augaccuaugaauugacagac

SEQ ID NO: 136 - RNA, 21 nt, Homo sapiens
uaaucucagcuggcaacugug

SEQ ID NO: 137 - RNA, 24 nt, Homo sapiens
uacugcaucaggaacugauuggau

SEQ ID NO: 138 - RNA, 21 nt, Homo sapiens
uugugcuugaucuaaccaugu

SEQ ID NO: 139 - RNA, 21 nt, Homo sapiens
ugauuguccaaacgcaauucu

SEQ ID NO: 140 - RNA, 21 nt, Homo sapiens
ccacaccguaucugacacuuu

SEQ ID NO: 141 - RNA, 23 nt, Homo sapiens
agcuacauugucugcuggguuuc

SEQ ID NO: 142 - RNA, 24 nt, Homo sapiens
agcuacaucuggcuacugggucuc

SEQ ID NO: 143 - RNA, 21 nt, Homo sapiens
agggcccccccucaauccugu

SEQ ID NO: 144 - RNA, 23 nt, Homo sapiens
cagugcaauaguauugucaaagc

SEQ ID NO: 145 - RNA, 23 nt, Homo sapiens
uaagugcuuccauguuuugguga

SEQ ID NO: 146 - RNA, 22 nt, Homo sapiens
uaaacguggauguacuugcuuu

SEQ ID NO: 147 - RNA, 23 nt, Homo sapiens
uaagugcuuccauguuuuaguag

SEQ ID NO: 148 - RNA, 23 nt, Homo sapiens
acuuuaacauggaagugcuuucu

SEQ ID NO: 149 - RNA, 23 nt, Homo sapiens
uaagugcuuccauguuucagugg

SEQ ID NO: 150 - RNA, 22 nt, Homo sapiens
uuuaacauggggguaccugcug

SEQ ID NO: 151 - RNA, 23 nt, Homo sapiens
uaagugcuuccauguuugagugu

SEQ ID NO: 152 - RNA, 23 nt, Homo sapiens
aaaagcuggguugagagggcgaa

SEQ ID NO: 153 - RNA, 22 nt, Homo sapiens
gcacauuacacggucgaccucu

SEQ ID NO: 154 - RNA, 23 nt, Homo sapiens
cgcauccccuagggcauuggugu

SEQ ID NO: 155 - RNA, 23 nt, Homo sapiens
ccuaguagguguccaguaagugu

SEQ ID NO: 156 - RNA, 20 nt, Homo sapiens
ccucugggcccuuccuccag

SEQ ID NO: 157 - RNA, 22 nt, Homo sapiens
cuggcccucucugcccuuccgu

SEQ ID NO: 158 - RNA, 23 nt, Homo sapiens
gcaaagcacacggccugcagaga

SEQ ID NO: 159 - RNA, 21 nt, Homo sapiens
gccccugggccuauccuagaa

SEQ ID NO: 160 - RNA, 23 nt, Homo sapiens
ucaagagcaauaacgaaaaaugu

SEQ ID NO: 161 - RNA, 23 nt, Homo sapiens
uccagcuccuauaugaugccuuu

SEQ ID NO: 162 - RNA, 23 nt, Homo sapiens
uccagcaucagugauuuuguuga

SEQ ID NO: 163 - RNA, 21 nt, Homo sapiens
ucccuguccuccaggagcuca

SEQ ID NO: 164 - RNA, 23 nt, Homo sapiens
uccgucucaguuacuuuauagcc

SEQ ID NO: 165 - RNA, 24 nt, Homo sapiens
ucucacacagaaaucgcacccguc

SEQ ID NO: 166 - RNA, 21 nt, Homo sapiens
ugcugacuccuaguccagggc

SEQ ID NO: 167 - RNA, 23 nt, Homo sapiens
ugucugcccgcaugccugccucu

SEQ ID NO: 168 - RNA, 22 nt, Homo sapiens
uuaucagaaucuccagggguac

SEQ ID NO: 169 - RNA, 22 nt, Homo sapiens
aauugcacuuuagcaaugguga

SEQ ID NO: 170 - RNA, 22 nt, Homo sapiens
acauagaggaaauuccacguuu

SEQ ID NO: 171 - RNA, 21 nt, Homo sapiens
aauaauacaugguugaucuuu

SEQ ID NO: 172 - RNA, 21 nt, Homo sapiens
gccugcugggguggaaccugg

SEQ ID NO: 173 - RNA, 21 nt, Homo sapiens
gugccgccaucuuuugagugu

SEQ ID NO: 174 - RNA, 23 nt, Homo sapiens
aaagugcugcgacauuugagcgu

SEQ ID NO: 175 - RNA, 23 nt, Homo sapiens
gaagugcuucgauuuuggggugu

SEQ ID NO: 176 - RNA, 22 nt, Homo sapiens
acucaaaaugggggcgcuuucc

SEQ ID NO: 177 - RNA, 22 nt, Homo sapiens
uuauaauacaaccugauaagug

SEQ ID NO: 178 - RNA, 22 nt, Homo sapiens
uuuguucguucggcucgcguga

SEQ ID NO: 179 - RNA, 21 nt, Homo sapiens
aucauagaggaaaauccacgu

SEQ ID NO: 180 - RNA, 22 nt, Homo sapiens
aucacacaaaggcaacuuuugu

SEQ ID NO: 181 - RNA, 22 nt, Homo sapiens
cuccugacuccagguccugugu

SEQ ID NO: 182 - RNA, 19 nt, Homo sapiens
ugguagacuauggaacgua

SEQ ID NO: 183 - RNA, 22 nt, Homo sapiens
uauguaauaugguccacaucuu

SEQ ID NO: 184 - RNA, 22 nt, Homo sapiens
ugguugaccauagaacaugcgc

SEQ ID NO: 185 - RNA, 22 nt, Homo sapiens
uauacaagggcaagcucucugu

SEQ ID NO: 186 - RNA, 22 nt, Homo sapiens
gaaguuguucgugguggauucg

SEQ ID NO: 187 - RNA, 22 nt, Homo sapiens
agaucagaaggugauuguggcu

SEQ ID NO: 188 - RNA, 20 nt, Homo sapiens
auuccuagaaauuguucaua

SEQ ID NO: 189 - RNA, 22 nt, Homo sapiens
cuggacuuggagucagaaggcc

SEQ ID NO: 190 - RNA, 22 nt, Homo sapiens
agcucggucugaggccccucag

SEQ ID NO: 191 - RNA, 22 nt, Homo sapiens
ugagguaguaaguuguauuguu

SEQ ID NO: 192 - RNA, 24 nt, Homo sapiens
aaaagugcuuacagugcagguagc

SEQ ID NO: 193 - RNA, 22 nt, Homo sapiens
ccacugccccaggugcugcugg

SEQ ID NO: 194 - RNA, 23 nt, Homo sapiens
uaaagugcuuauagugcagguag

SEQ ID NO: 195 - RNA, 19 nt, Homo sapiens
gguccagaggggagauagg

SEQ ID NO: 196 - RNA, 23 nt, Homo sapiens
ucuuugguuaucuagcuguauga

SEQ ID NO: 197 - RNA, 21 nt, Homo sapiens
uaaagcuagauaaccgaaagu

SEQ ID NO: 198 - RNA, 22 nt, Homo sapiens
uagcaccauuugaaaucgguua

SEQ ID NO: 199 - RNA, 22 nt, Homo sapiens
ucacagugaaccggucucuuuc

SEQ ID NO: 200 - RNA, 21 nt, Homo sapiens
cuuuuugcggucugggcuugc

SEQ ID NO: 201 - RNA, 21 nt, Homo sapiens
uugguccccuucaaccagcua

SEQ ID NO: 202 - RNA, 23 nt, Homo sapiens
acuccauuuguuuugaugaugga

SEQ ID NO: 203 - RNA, 22 nt, Homo sapiens
uauugcuuaagaauacgcguag

SEQ ID NO: 204 - RNA, 17 nt, Homo sapiens
agcugguguugugaauc

SEQ ID NO: 205 - RNA, 22 nt, Homo sapiens
acuagacugaagcuccuugagg

SEQ ID NO: 206 - RNA, 22 nt, Homo sapiens
uuuggcaaugguagaacucaca

SEQ ID NO: 207 - RNA, 18 nt, Homo sapiens
uggagagaaaggcaguuc

SEQ ID NO: 208 - RNA, 23 nt, Homo sapiens
caagucacuagugguuccguuua

SEQ ID NO: 209 - RNA, 22 nt, Homo sapiens
ugguuuaccgucccacauacau

SEQ ID NO: 210 - RNA, 22 nt, Homo sapiens
uguaaacauccuacacucagcu

SEQ ID NO: 211 - RNA, 22 nt, Homo sapiens
cuggacuuagggucagaaggcc

SEQ ID NO: 212 - RNA, 22 nt, Homo sapiens
cagcagcaauucauguuuugaa

SEQ ID NO: 213 - RNA, 42 nt, Homo sapiens
cgcaaggaugacacgcaaauucgugaagcguuccauauuuuu

SEQ ID NO: 214 - RNA, 22 nt, Homo sapiens
uggguggucuggagauuugugc

SEQ ID NO: 215 - RNA, 18 nt, Homo sapiens
uccagugcccuccucucc

SEQ ID NO: 216 - RNA, 22 nt, Homo sapiens
uuagggcccuggcuccaucucc

SEQ ID NO: 217 - RNA, 22 nt, Homo sapiens
aaaaguaauugcggauuuugcc

SEQ ID NO: 218 - RNA, 22 nt, Homo sapiens
caucccuugcaugguggagggu

SEQ ID NO: 219 - RNA, 22 nt, Homo sapiens
uaaggugcaucuagugcaguua

SEQ ID NO: 220 - RNA, 24 nt, Homo sapiens
aacuggcccucaaagucccgcuuu

SEQ ID NO: 221 - RNA, 22 nt, Homo sapiens
caucuuaccggacagugcugga

SEQ ID NO: 222 - RNA, 22 nt, Homo sapiens
agagguauagggcaugggaaaa

SEQ ID NO: 223 - RNA, 22 nt, Homo sapiens
uuuccuaugcauauacuucuuu

SEQ ID NO: 224 - RNA, 23 nt, Homo sapiens
caaagugcucauagugcagguag

SEQ ID NO: 225 - RNA, 22 nt, Homo sapiens
uaugugggaugguaaaccgcuu

SEQ ID NO: 226 - RNA, 22 nt, Homo sapiens
uaaugccccuaaaaauccuuau

SEQ ID NO: 227 - RNA, 22 nt, Homo sapiens
agaucgaccguguuauauucgc

SEQ ID NO: 228 - RNA, 22 nt, Homo sapiens
agguuacccgagcaacuuugca

SEQ ID NO: 229 - RNA, 23 nt, Homo sapiens
acuucaccugguccacuagccgu

SEQ ID NO: 230 - RNA, 22 nt, Homo sapiens
uaauacugucugguaaaaccgu

SEQ ID NO: 231 - RNA, 23 nt, Homo sapiens
ucuuggaguaggucauugggugg

SEQ ID NO: 232 - RNA, 21 nt, Homo sapiens
cuggauggcuccuccaugucu

SEQ ID NO: 233 - RNA, 22 nt, Homo sapiens
aucaugaugggcuccucggugu

SEQ ID NO: 234 - RNA, 22 nt, Homo sapiens
uugcauauguaggaugucccau

SEQ ID NO: 235 - RNA, 22 nt, Homo sapiens
uggcaguguauuguuagcuggu

SEQ ID NO: 236 - RNA, 22 nt, Homo sapiens
uuuuugcgauguguuccuaaua

SEQ ID NO: 237 - RNA, 22 nt, Homo sapiens
uguuugcagaggaaacugagac

SEQ ID NO: 238 - RNA, 21 nt, Homo sapiens
ucagucucaucugcaaagaag

SEQ ID NO: 239 - RNA, 22 nt, Homo sapiens
gagguuguccguggugaguucg

SEQ ID NO: 240 - RNA, 22 nt, Homo sapiens
agaggcuggccgugaugaauuc

SEQ ID NO: 241 - RNA, 22 nt, Homo sapiens
caaccuggaggacuccaugcug

SEQ ID NO: 242 - RNA, 23 nt, Homo sapiens
aguggggaacccuuccaugagga

SEQ ID NO: 243 - RNA, 22 nt, Homo sapiens
ucuauacagacccuggcuuuuc

SEQ ID NO: 244 - RNA, 22 nt, Homo sapiens
aaaaguauuugcggguuuuguc

SEQ ID NO: 245 - RNA, 23 nt, Homo sapiens
aggaccugcgggacaagauucuu

SEQ ID NO: 246 - RNA, 22 nt, Homo sapiens
uuguacaugguaggcuuucauu

SEQ ID NO: 247 - RNA, 24 nt, Homo sapiens
ugaaacauacacgggaaaccucuu

SEQ ID NO: 248 - RNA, 17 nt, Homo sapiens
auuacauggccaaucuc

SEQ ID NO: 249 - RNA, 21 nt, Homo sapiens
cagcagcacacugugguuugu

SEQ ID NO: 250 - RNA, 23 nt, Homo sapiens
uuucaagccagggggcguuuuuc

SEQ ID NO: 251 - RNA, 23 nt, Homo sapiens
uuaagacuugcagugauguuuaa

SEQ ID NO: 252 - RNA, 22 nt, Homo sapiens
augcaccugggcaaggauucug

SEQ ID NO: 253 - RNA, 22 nt, Homo sapiens
aauccuuugucccugggugaga

SEQ ID NO: 254 - RNA, 23 nt, Homo sapiens
uagcagcgggaacaguucugcag

SEQ ID NO: 255 - RNA, 22 nt, Homo sapiens
gucaacacuugcugguuuccuc

SEQ ID NO: 256 - RNA, 21 nt, Homo sapiens
uaaggcacccuucugaguaga

SEQ ID NO: 257 - RNA, 21 nt, Homo sapiens
uuuugcaccuuuuggagugaa

SEQ ID NO: 258 - RNA, 23 nt, Homo sapiens
ugauuguagccuuuuggaguaga

SEQ ID NO: 259 - RNA, 23 nt, Homo sapiens
ugauugguacgucuguggguaga

SEQ ID NO: 260 - RNA, 67 nt, Homo sapiens
ugguauugccauugcuucacuguuggcuuugaccaggguaugaucucuuaaucuucucucugagcug

SEQ ID NO: 261 - RNA, 42 nt, Homo sapiens; identical to SEQ ID NO: 213

SEQ ID NO: 262 - RNA, 60 nt, Homo sapiens
ccuggaugaugauagcaaaugcugacugaacaugaaggucuuaauuagcucuaacugacu

SEQ ID NO: 263 - RNA, 52 nt, Homo sapiens
gaacuuauugacgggcggacagaaacugugugcugauugucacguucugauu

SEQ ID NO: 264 - RNA, 18 nt, Homo sapiens
ucuacagugcacgugucu

SEQ ID NO: 265 - RNA, 22 nt, Homo sapiens
aacauucauugcugucgguggg

SEQ ID NO: 266 - RNA, 24 nt, Homo sapiens
aacauucauuguugucgguggguu

SEQ ID NO: 267 - RNA, 21 nt, Homo sapiens
ggcaagaugcuggcauagcug

SEQ ID NO: 268 - RNA, 22 nt, Homo sapiens
aacacaccugguuaaccucuuu

SEQ ID NO: 269 - RNA, 22 nt, Homo sapiens
aucauagaggaaaauccauguu

SEQ ID NO: 270 - RNA, 21 nt, Homo sapiens
aucgggaaugucguguccgcc

SEQ ID NO: 271 - RNA, 23 nt, Homo sapiens
aaaccguuaccauuacugaguuu

SEQ ID NO: 272 - RNA, 21 nt, Homo sapiens
cccagauaauggcacucucaa

SEQ ID NO: 273 - RNA, 23 nt, Homo sapiens
agugacaucacauauacggcagc

SEQ ID NO: 274 - RNA, 23 nt, Homo sapiens
aaacaaacauggugcacuucuuu

SEQ ID NO: 275 - RNA, 21 nt, Homo sapiens
auccuugcuaucugggugcua

SEQ ID NO: 276 - RNA, 21 nt, Homo sapiens
agacccuggucugcacucuau

SEQ ID NO: 277 - RNA, 21 nt, Homo sapiens
gugucuuuugcucugcaguca

SEQ ID NO: 278 - RNA, 24 nt, Homo sapiens
uucuccaaaagaaagcacuuucug

SEQ ID NO: 279 - RNA, 22 nt, Homo sapiens
ccucuagauggaagcacugucu

SEQ ID NO: 280 - RNA, 23 nt, Homo sapiens
aaagugcauccuuuuagagguuu

SEQ ID NO: 281 - RNA, 21 nt, Homo sapiens
aaagugcuuccuuuuagaggg

SEQ ID NO: 282 - RNA, 23 nt, Homo sapiens
aaagugcuuccuuuuagaggguu

SEQ ID NO: 283 - RNA, 23 nt, Homo sapiens
aaagugcuucucuuugguggguu

SEQ ID NO: 284 - RNA, 21 nt, Homo sapiens
aaagugcuuccuuuuugaggg

SEQ ID NO: 285 - RNA, 22 nt, Homo sapiens
aagugcuuccuuuuagaggguu

SEQ ID NO: 286 - RNA, 24 nt, Homo sapiens
acaaagugcuucccuuuagagugu

SEQ ID NO: 287 - RNA, 22 nt, Homo sapiens
aacgcacuucccuuuagagugu

SEQ ID NO: 288 - RNA, 21 nt, Homo sapiens
gaaggcgcuucccuuuagagc

SEQ ID NO: 289 - RNA, 21 nt, Homo sapiens
cucuagagggaagcacuuucu

SEQ ID NO: 290 - RNA, 21 nt, Homo sapiens
aaagugcuuccuuuuagaggc

SEQ ID NO: 291 - RNA, 23 nt, Homo sapiens
uacucaggagaguggcaaucaca

SEQ ID NO: 292 - RNA, 23 nt, Homo sapiens
cacucagccuugagggcacuuuc

SEQ ID NO: 293 - RNA, 22 nt, Homo sapiens
uucacagggaggugucauuuau

SEQ ID NO: 294 - RNA, 20 nt, Homo sapiens
auugacacuucugugaguag

SEQ ID NO: 295 - RNA, 21 nt, Homo sapiens
gagugccuucuuuuggagcgu

SEQ ID NO: 296 - RNA, 18 nt, Homo sapiens
ugcuuccuuucagagggu

SEQ ID NO: 297 - RNA, 22 nt, Homo sapiens
aucuggagguaagaagcacuuu

SEQ ID NO: 298 - RNA, 23 nt, Homo sapiens
aucgugcaucccuuuagaguguu

SEQ ID NO: 299 - RNA, 22 nt, Homo sapiens
ucgugcaucccuuuagaguguu

SEQ ID NO: 300 - RNA, 22 nt, Homo sapiens
aucgugcauccuuuuagagugu

SEQ ID NO: 301 - RNA, 21 nt, Homo sapiens
aaagcgcuucccuuugcugga

SEQ ID NO: 302 - RNA, 22 nt, Homo sapiens
caaagcgcuccccuuuagaggu

SEQ ID NO: 303 - RNA, 22 nt, Homo sapiens
caaagcgcuucucuuuagagug

SEQ ID NO: 304 - RNA, 23 nt, Homo sapiens
ucucuggagggaagcacuuucug

SEQ ID NO: 305 - RNA, 21 nt, Homo sapiens
caaagcgcuucccuuuggagc

SEQ ID NO: 306 - RNA, 22 nt, Homo sapiens
aaagcgcuucccuucagagugu

SEQ ID NO: 307 - RNA, 21 nt, Homo sapiens
aaagcgcuucucuuuagagga

SEQ ID NO: 308 - RNA, 25 nt, Homo sapiens
aaagugcauccuuuuagaguguuac

SEQ ID NO: 309 - RNA, 22 nt, Homo sapiens
aaagugcaucuuuuuagaggau

SEQ ID NO: 310 - RNA, 23 nt, Homo sapiens
caaagugccucccuuuagagugu

SEQ ID NO: 311 - RNA, 22 nt, Homo sapiens
aaagugccuccuuuuagagugu

SEQ ID NO: 312 - RNA, 22 nt, Homo sapiens
uucuccaaaagggagcacuuuc

SEQ ID NO: 313 - RNA, 22 nt, Homo sapiens
aaagugcuucccuuuggacugu

SEQ ID NO: 314 - RNA, 21 nt, Homo sapiens
cuccagagggaaguacuuucu

SEQ ID NO: 315 - RNA, 23 nt, Homo sapiens
ucuacaaagggaagcccuuucug

SEQ ID NO: 316 - RNA, 22 nt, Homo sapiens
acaaagugcuucccuuuagagu

SEQ ID NO: 317 - RNA, 23 nt, Homo sapiens
aaaaugguucccuuuagaguguu

SEQ ID NO: 318 - RNA, 21 nt, Homo sapiens
aacgcgcuucccuauagaggg

SEQ ID NO: 319 - RNA, 21 nt, Homo sapiens
gaaggcgcuucccuuuggagu

SEQ ID NO: 320 - RNA, 21 nt, Homo sapiens
cuccagagggaugcacuuucu

SEQ ID NO: 321 - RNA, 24 nt, Homo sapiens
cucuugagggaagcacuuucuguu

SEQ ID NO: 322 - RNA, 24 nt, Homo sapiens
cucuagagggaagcgcuuucuguu

SEQ ID NO: 323 - RNA, 21 nt, Homo sapiens
cugcaaagggaagcccuuucu

SEQ ID NO: 324 - RNA, 70 nt, Homo sapiens
caguagugaugaaauuccacuucauugguccguguuucugaaccacaugauuuucucggauguucugaug

SEQ ID NO: 325 - RNA, 66 nt, Homo sapiens
cugcgaugauggcauuucuuaggacaccuuuggauuaauaaugaaaacaacuacucucugagcagc

SEQ ID NO: 326 - RNA, 65 nt, Homo sapiens
cugcagugaugacuuucuugggacaccuuuggauuuaccgugaaaauuaauaaauucugagcagc

SEQ ID NO: 327 - RNA, 70 nt, Homo sapiens
cuuaaugaugacuguuuuuuuugauugcuugaagcaaugugaaaaacacauuucaccggcucugaaagcu

SEQ ID NO: 328 - RNA, 63 nt, Homo sapiens
uggcgaugaggagguaccuauuguguugaguaacggugauaauuuuauacgcuauucugagcc

SEQ ID NO: 329 - RNA, 57 nt, Homo sapiens
ccagucacagauuucuuuguuccuucuccacucccacugcaucacuuaacuagccuu

SEQ ID NO: 330 - RNA, 60 nt, Homo sapiens
agccugugaugcuuuaagaguaguggacagaagggauuucugaaauucuauucugaggcu

SEQ ID NO: 331 - RNA, 65 nt, Homo sapiens
uaaugauucugccaaaugaaauauaaugauaucacuguaaaaccguuccauuuugauucugaggu

SEQ ID NO: 332 - RNA, 22 nt, Homo sapiens
aauugcacgguauccaucugua

SEQ ID NO: 333 - RNA, 21 nt, Homo sapiens
acugcccuaagugcuccuucu

SEQ ID NO: 334 - RNA, 24 nt, Homo sapiens
aauccuuggaaccuaggugugagu

SEQ ID NO: 335 - RNA, 21 nt, Homo sapiens
aauauaacacagauggccugu

SEQ ID NO: 336 - RNA, 23 nt, Homo sapiens
ucacuccucuccucccgucuucu

SEQ ID NO: 337 - RNA, 22 nt, Homo sapiens
gucauacacggcucuccucucu

SEQ ID NO: 338 - RNA, 22 nt, Homo sapiens
uccuguacugagcugccccgag

SEQ ID NO: 339 - RNA, 22 nt, Homo sapiens
aaucauacagggacauccaguu

SEQ ID NO: 340 - RNA, 22 nt, Homo sapiens
uaugugccuuuggacuacaucg

SEQ ID NO: 341 - RNA, 23 nt, Homo sapiens
caucuggagguaagaagcacuuu

SEQ ID NO: 342 - RNA, 21 nt, Homo sapiens
ugaaggucuacugugugccag

SEQ ID NO: 343 - RNA, 22 nt, Homo sapiens
cggguggaucacgaugcaauuu

SEQ ID NO: 344 - RNA, 22 nt, Homo sapiens
ugugacagauugauaacugaaa

SEQ ID NO: 345 - RNA, 22 nt, Homo sapiens
aaucguacagggucauccacuu

SEQ ID NO: 346 - RNA, 22 nt, Homo sapiens
ggagaaauuauccuuggugugu

SEQ ID NO: 347 - RNA, 21 nt, Homo sapiens
gguagauucuccuucuaugag

SEQ ID NO: 348 - RNA, 22 nt, Homo sapiens
ucggggaucaucaugucacgag

SEQ ID NO: 349 - RNA, 22 nt, Homo sapiens
aucagcaaacauuuauugugug

SEQ ID NO: 350 - RNA, 20 nt, Homo sapiens
auucugcauuuuuagcaagu

SEQ ID NO: 351 - RNA, 21 nt, Homo sapiens
aauauuauacagucaaccucu

SEQ ID NO: 352 - RNA, 21 nt, Homo sapiens
ugacaacuauggaugagcucu

SEQ ID NO: 353 - RNA, 23 nt, Homo sapiens
ggcagguucucacccucucuagg

SEQ ID NO: 354 - RNA, 25 nt, Homo sapiens
ggcggagggaaguagguccguuggu

SEQ ID NO: 355 - RNA, 22 nt, Homo sapiens
cuugguucagggagggucccca

SEQ ID NO: 356 - RNA, 22 nt, Homo sapiens
uacccauugcauaucggaguug

SEQ ID NO: 357 - RNA, 23 nt, Homo sapiens
aaugacacgaucacucccguuga

SEQ ID NO: 358 - RNA, 23 nt, Homo sapiens
aauggcgccacuaggguugugca

SEQ ID NO: 359 - RNA, 22 nt, Homo sapiens
caugccuugaguguaggaccgu

SEQ ID NO: 360 - RNA, 21 nt, Homo sapiens
gcgacccacucuugguuucca

SEQ ID NO: 361 - RNA, 21 nt, Homo sapiens
aacaggugacugguuagacaa

SEQ ID NO: 362 - RNA, 21 nt, Homo sapiens
aaaacggugagauuuuguuuu

SEQ ID NO: 363 - RNA, 21 nt, Homo sapiens
gcuaguccugacucagccagu

SEQ ID NO: 364 - RNA, 21 nt, Homo sapiens
aggguaagcugaaccucugau

SEQ ID NO: 365 - RNA, 20 nt, Homo sapiens
gaugagcucauuguaauaug

SEQ ID NO: 366 - RNA, 23 nt, Homo sapiens
guuugcacgggugggccuugucu

SEQ ID NO: 367 - RNA, 19 nt, Homo sapiens
ugagcugcuguaccaaaau

SEQ ID NO: 368 - RNA, 21 nt, Homo sapiens
uaaaguaaauaugcaccaaaa

SEQ ID NO: 369 - RNA, 22 nt, Homo sapiens
caaaguuuaagauccuugaagu

SEQ ID NO: 370 - RNA, 20 nt, Homo sapiens
aaaguagcuguaccauuugc

SEQ ID NO: 371 - RNA, 19 nt, Homo sapiens
agguugacauacguuuccc

SEQ ID NO: 372 - RNA, 19 nt, Homo sapiens
aggcacggugucagcaggc

SEQ ID NO: 373 - RNA, 22 nt, Homo sapiens
ggcuggcucgcgaugucuguuu

SEQ ID NO: 374 - RNA, 19 nt, Homo sapiens
gggcgccugugaucccaac

SEQ ID NO: 375 - RNA, 23 nt, Homo sapiens
aguauguucuuccaggacagaac

SEQ ID NO: 376 - RNA, 21 nt, Homo sapiens
gcgacccauacuugguuucag

SEQ ID NO: 377 - RNA, 21 nt, Homo sapiens
aguuaaugaauccuggaaagu

SEQ ID NO: 378 - RNA, 22 nt, Homo sapiens
gaaaacagcaauuaccuuugca

SEQ ID NO: 379 - RNA, 22 nt, Homo sapiens
caaaacuggcaauuacuuuugc

SEQ ID NO: 380 - RNA, 22 nt, Homo sapiens
uaugcauuguauuuuuaggucc

SEQ ID NO: 381 - RNA, 21 nt, Homo sapiens
uuuccauaggugaugagucac

SEQ ID NO: 382 - RNA, 22 nt, Homo sapiens
caagaaccucaguugcuuuugu

SEQ ID NO: 383 - RNA, 21 nt, Homo sapiens
uuggccacaauggguuagaac

SEQ ID NO: 384 - RNA, 24 nt, Homo sapiens
ucagaacaaaugccgguucccaga

SEQ ID NO: 385 - RNA, 22 nt, Homo sapiens
ugucuuacucccucaggcacau

SEQ ID NO: 386 - RNA, 20 nt, Homo sapiens
agaccauggguucucauugu

SEQ ID NO: 387 - RNA, 22 nt, Homo sapiens
uugugucaauaugcgaugaugu

SEQ ID NO: 388 - RNA, 25 nt, Homo sapiens
aggcaccagccaggcauugcucagc

SEQ ID NO: 389 - RNA, 25 nt, Homo sapiens
cccaucugggguggccugugacuuu

SEQ ID NO: 390 - RNA, 21 nt, Homo sapiens
aagccugcccggcuccucggg

SEQ ID NO: 391 - RNA, 22 nt, Homo sapiens
ugugucacucgaugaccacugu

SEQ ID NO: 392 - RNA, 21 nt, Homo sapiens
acagucugcugagguuggagc

SEQ ID NO: 393 - RNA, 20 nt, Homo sapiens
guugugucaguuuaucaaac

SEQ ID NO: 394 - RNA, 23 nt, Homo sapiens
aucccuugcaggggcuguugggu

SEQ ID NO: 395 - RNA, 23 nt, Homo sapiens
acuuacagacaagagccuugcuc

SEQ ID NO: 396 - RNA, 22 nt, Homo sapiens
uaguaccaguaccuuguguuca

SEQ ID NO: 397 - RNA, 22 nt, Homo sapiens
uggucuaggauuguuggaggag

SEQ ID NO: 398 - RNA, 19 nt, Homo sapiens
agcugucugaaaaugucuu

SEQ ID NO: 399 - RNA, 22 nt, Homo sapiens
gugagucucuaagaaaagagga

SEQ ID NO: 400 - RNA, 20 nt, Homo sapiens
ucuaguaagaguggcagucg

SEQ ID NO: 401 - RNA, 22 nt, Homo sapiens
guucucccaacguaagcccagc

SEQ ID NO: 402 - RNA, 22 nt, Homo sapiens
aguauucuguaccagggaaggu

SEQ ID NO: 403 - RNA, 21 nt, Homo sapiens
agaccuggcccagaccucagc

SEQ ID NO: 404 - RNA, 21 nt, Homo sapiens
gugcauugcuguugcauugca

SEQ ID NO: 405 - RNA, 22 nt, Homo sapiens
cacacacugcaauuacuuuugc

SEQ ID NO: 406 - RNA, 19 nt, Homo sapiens
aggcugcggaauucaggac

SEQ ID NO: 407 - RNA, 23 nt, Homo sapiens
uaaaucccauggugccuucuccu

SEQ ID NO: 408 - RNA, 21 nt, Homo sapiens
aaacuacugaaaaucaaagau

SEQ ID NO: 409 - RNA, 21 nt, Homo sapiens
guucaaauccagaucuauaac

SEQ ID NO: 410 - RNA, 25 nt, Homo sapiens
aggggugguguugggacagcuccgu

SEQ ID NO: 411 - RNA, 19 nt, Homo sapiens
gugucugcuuccuguggga

SEQ ID NO: 412 - RNA, 20 nt, Homo sapiens
aggguguuucucucaucucu

SEQ ID NO: 413 - RNA, 23 nt, Homo sapiens
cuaauaguaucuaccacaauaaa

SEQ ID NO: 414 - RNA, 21 nt, Homo sapiens
ugagcuaaaugugugcuggga

SEQ ID NO: 415 - RNA, 22 nt, Homo sapiens
aaccagcaccccaacuuuggac

SEQ ID NO: 416 - RNA, 23 nt, Homo sapiens
acuugggcacugaaacaaugucc

SEQ ID NO: 417 - RNA, 25 nt, Homo sapiens
gcugggcagggcuucugagcuccuu

SEQ ID NO: 418 - RNA, 24 nt, Homo sapiens
ugugcuugcucgucccgcccgcag

SEQ ID NO: 419 - RNA, 24 nt, Homo sapiens
acugggggcuuucgggcucugcgu

SEQ ID NO: 420 - RNA, 25 nt, Homo sapiens
agggaucgcgggcggguggcggccu

SEQ ID NO: 421 - RNA, 23 nt, Homo sapiens
aucgcugcgguugcgagcgcugu

SEQ ID NO: 422 - RNA, 21 nt, Homo sapiens
augauccaggaaccugccucu

SEQ ID NO: 423 - RNA, 24 nt, Homo sapiens
aaagacauaggauagagucaccuc

SEQ ID NO: 424 - RNA, 20 nt, Homo sapiens
aggaauguuccuucuuugcc

SEQ ID NO: 425 - RNA, 23 nt, Homo sapiens
gaacgccuguucuugccaggugg

SEQ ID NO: 426 - RNA, 21 nt, Homo sapiens
uccgagccugggucucccucu

SEQ ID NO: 427 - RNA, 22 nt, Homo sapiens
acucaaaacccuucagugacuu

SEQ ID NO: 428 - RNA, 22 nt, Homo sapiens
caaaaaucucaauuacuuuugc

SEQ ID NO: 429 - RNA, 22 nt, Homo sapiens
agacuucccauuugaagguggc

SEQ ID NO: 430 - RNA, 22 nt, Homo sapiens
gucccucuccaaaugugucuug

SEQ ID NO: 431 - RNA, 23 nt, Homo sapiens
aaacucuacuuguccuucugagu

SEQ ID NO: 432 - RNA, 22 nt, Homo sapiens
acuuguaugcuagcucagguag

SEQ ID NO: 433 - RNA, 24 nt, Homo sapiens
gaccuggacauguuugugcccagu

SEQ ID NO: 434 - RNA, 19 nt, Homo sapiens
aguguggcuuucuuagagc

SEQ ID NO: 435 - RNA, 19 nt, Homo sapiens
ucuaggcugguacugcuga

SEQ ID NO: 436 - RNA, 21 nt, Homo sapiens
ggcuagcaacagcgcuuaccu

SEQ ID NO: 437 - RNA, 19 nt, Homo sapiens
aagcagcugccucugaggc

SEQ ID NO: 438 - RNA, 21 nt, Homo sapiens
guggcugcacucacuuccuuc

SEQ ID NO: 439 - RNA, 19 nt, Homo sapiens
aagugugcagggcacuggu

SEQ ID NO: 440 - RNA, 22 nt, Homo sapiens
aaaccuguguuguucaagaguc

SEQ ID NO: 441 - RNA, 21 nt, Homo sapiens
aggaggcagcgcucucaggac

SEQ ID NO: 442 - RNA, 22 nt, Homo sapiens
uuuaggauaagcuugacuuuug

SEQ ID NO: 443 - RNA, 22 nt, Homo sapiens
caaaaaccacaguuucuuuugc

SEQ ID NO: 444 - RNA, 24 nt, Homo sapiens
ugccugggucucuggccugcgcgu

SEQ ID NO: 445 - RNA, 21 nt, Homo sapiens
ucccacguuguggcccagcag

SEQ ID NO: 446 - RNA, 22 nt, Homo sapiens
aggcaguguauuguuagcuggc

SEQ ID NO: 447 - RNA, 21 nt, Homo sapiens
uugaaacaaucucuacugaac

SEQ ID NO: 448 - RNA, 21 nt, Homo sapiens
uaguagaccguauagcguacg

SEQ ID NO: 449 - RNA, 22 nt, Homo sapiens
uggugggccgcagaacaugugc

SEQ ID NO: 450 - RNA, 22 nt, Homo sapiens
auaauacaugguuaaccucuuu

SEQ ID NO: 451 - RNA, 21 nt, Homo sapiens
ugaguuggccaucugagugag

SEQ ID NO: 452 - RNA, 20 nt, Homo sapiens
guccgcucggcgguggccca

SEQ ID NO: 453 - RNA, 24 nt, Homo sapiens
cugaagugauguguaacugaucag

SEQ ID NO: 454 - RNA, 19 nt, Homo sapiens
gagccaguuggacaggagc

SEQ ID NO: 455 - RNA, 23 nt, Homo sapiens
auucuaauuucuccacgucuuug

SEQ ID NO: 456 - RNA, 21 nt, Homo sapiens
cuucuugugcucuaggauugu

SEQ ID NO: 457 - RNA, 23 nt, Homo sapiens
auucauuugguauaaaccgcgau

SEQ ID NO: 458 - RNA, 22 nt, Homo sapiens
uugagaaugaugaaucauuagg

SEQ ID NO: 459 - RNA, 21 nt, Homo sapiens
ucuuguguucucuagaucagu

SEQ ID NO: 460 - RNA, 21 nt, Homo sapiens
caaagaggaaggucccauuac

SEQ ID NO: 461 - RNA, 22 nt, Homo sapiens
uuaugguuugccugggacugag

SEQ ID NO: 462 - RNA, 19 nt, Homo sapiens
ugggcguaucuguaugcua

SEQ ID NO: 463 - RNA, 65 nt, Homo sapiens
uggcagugaugaucacaaauccguguuucugacaagcgauugacgauagaaaaccggcugagcca

SEQ ID NO: 464 - RNA, 23 nt, Homo sapiens
uaauacugccugguaaugaugac

SEQ ID NO: 465 - RNA, 22 nt, Homo sapiens
ucaggcucaguccccucccgau

SEQ ID NO: 466 - RNA, 22 nt, Homo sapiens
aagugcugucauagcugagguc

SEQ ID NO: 467 - RNA, 21 nt, Homo sapiens
ugucuugcaggccgucaugca

SEQ ID NO: 468 - RNA, 22 nt, Homo sapiens
cuacaaagggaagcacuuucuc

SEQ ID NO: 469 - RNA, 23 nt, Homo sapiens
uuacaguuguucaaccaguuacu

SEQ ID NO: 470 - RNA, 22 nt, Homo sapiens
gagcuuauucauaaaagugcag

SEQ ID NO: 471 - RNA, 22 nt, Homo sapiens
acuccagccccacagccucagc

SEQ ID NO: 472 - RNA, 21 nt, Homo sapiens
gaagugugccguggugugucu

SEQ ID NO: 473 - RNA, 22 nt, Homo sapiens
uacgucaucguugucaucguca

SEQ ID NO: 474 - RNA, 22 nt, Homo sapiens
uuugugaccugguccacuaacc

SEQ ID NO: 475 - RNA, 23 nt, Homo sapiens
ugucacucggcucggcccacuac

SEQ ID NO: 476 - RNA, 23 nt, Homo sapiens
ugcaccaugguugucugagcaug

SEQ ID NO: 477 - RNA, 24 nt, Homo sapiens
gauugcucugcgugcggaaucgac

SEQ ID NO: 478 - RNA, 23 nt, Homo sapiens
ucugcucauaccccaugguuucu

SEQ ID NO: 479 - RNA, 22 nt, Homo sapiens
acccuaucaauauugucucugc

SEQ ID NO: 480 - RNA, 22 nt, Homo sapiens
ugagaccucuggguucugagcu

SEQ ID NO: 481 - RNA, 26 nt, Homo sapiens
guuggaggaugaaaguacggagugau

SEQ ID NO: 482 - RNA, 28 nt, Homo sapiens
ucacaaugcugacacucaaacugcugac

SEQ ID NO: 483 - RNA, 23 nt, Homo sapiens
uccaguaccacgugucagggcca

SEQ ID NO: 484 - RNA, 23 nt, Homo sapiens
cugggaucuccggggucuugguu

SEQ ID NO: 485 - RNA, 23 nt, Homo sapiens
caguaacaaagauucauccuugu

SEQ ID NO: 486 - RNA, 23 nt, Homo sapiens
uggugcggagagggcccacagug

SEQ ID NO: 487 - RNA, 22 nt, Homo sapiens
gcacugagaugggaguggugua

SEQ ID NO: 488 - RNA, 22 nt, Homo sapiens
aaugcaccugggcaaggauuca

SEQ ID NO: 489 - RNA, 22 nt, Homo sapiens
agacccuggucugcacucuauc

SEQ ID NO: 490 - RNA, 20 nt, Homo sapiens
gugcauugcuguugcauugc

SEQ ID NO: 491 - RNA, 22 nt, Homo sapiens
gggagccaggaaguauugaugu

SEQ ID NO: 492 - RNA, 23 nt, Homo sapiens
ugugcuugcucgucccgcccgca

SEQ ID NO: 493 - RNA, 22 nt, Homo sapiens
cgucaacacuugcugguuuccu

SEQ ID NO: 494 - RNA, 18 nt, Homo sapiens
uucacagggaggugucau

SEQ ID NO: 495 - RNA, 23 nt, Homo sapiens
uaaauuucaccuuucugagaagg

SEQ ID NO: 496 - RNA, 23 nt, Homo sapiens
uacuccagagggcgucacucaug

SEQ ID NO: 497 - RNA, 21 nt, Homo sapiens
cggggcagcucaguacaggau

SEQ ID NO: 498 - RNA, 22 nt, Homo sapiens
augguuccgucaagcaccaugg

SEQ ID NO: 499 - RNA, 22 nt, Homo sapiens
agaguugagucuggacgucccg

SEQ ID NO: 500 - RNA, 22 nt, Homo sapiens
accuggcauacaauguagauuu

SEQ ID NO: 501 - RNA, 22 nt, Homo sapiens
cucaguagccaguguagauccu

SEQ ID NO: 502 - RNA, 22 nt, Homo sapiens
cguguauuugacaagcugaguu

SEQ ID NO: 503 - RNA, 21 nt, Homo sapiens
caagucacuagugguuccguu

SEQ ID NO: 504 - RNA, 22 nt, Homo sapiens
caucaucgucucaaaugagucu

SEQ ID NO: 505 - RNA, 22 nt, Homo sapiens
gaggguuggguggaggcucucc

SEQ ID NO: 506 - RNA, 22 nt, Homo sapiens
caaucacuaacuccacugccau

SEQ ID NO: 507 - RNA, 22 nt, Homo sapiens
aggggcuggcuuuccucugguc

SEQ ID NO: 508 - RNA, 22 nt, Homo sapiens
gcccaaaggugaauuuuuuggg

SEQ ID NO: 509 - RNA, 21 nt, Homo sapiens
cucccacaugcaggguuugca

SEQ ID NO: 510 - RNA, 22 nt, Homo sapiens
ccaauauuggcugugcugcucc

SEQ ID NO: 511 - RNA, 22 nt, Homo sapiens
cugggagaggguuguuuacucc

SEQ ID NO: 512 - RNA, 22 nt, Homo sapiens
uauugcacauuacuaaguugca

SEQ ID NO: 513 - RNA, 22 nt, Homo sapiens
cugggagaaggcuguuuacucu

SEQ ID NO: 514 - RNA, 22 nt, Homo sapiens
caauuuagugugugugauauuu

SEQ ID NO: 515 - RNA, 22 nt, Homo sapiens
uagcaccaucugaaaucgguua

SEQ ID NO: 516 - RNA, 22 nt, Homo sapiens
ugcuaugccaacauauugccau

SEQ ID NO: 517 - RNA, 21 nt, Homo sapiens
acucuuucccuguugcacuac

SEQ ID NO: 518 - RNA, 22 nt, Homo sapiens
ccuauucuugauuacuuguuuc

SEQ ID NO: 519 - RNA, 23 nt, Homo sapiens
ucccccaggugugauucugauuu

SEQ ID NO: 520 - RNA, 22 nt, Homo sapiens
aacacaccuauucaaggauuca

SEQ ID NO: 521 - RNA, 21 nt, Homo sapiens
cuguacaggccacugccuugc

SEQ ID NO: 522 - RNA, 22 nt, Homo sapiens
acuuuaacauggaagugcuuuc

SEQ ID NO: 523 - RNA, 22 nt, Homo sapiens
acuuuaacauggaggcacuugc

SEQ ID NO: 524 - RNA, 22 nt, Homo sapiens
acuguugcuaauaugcaacucu

SEQ ID NO: 525 - RNA, 21 nt, Homo sapiens
aacauagaggaaauuccacgu

SEQ ID NO: 526 - RNA, 23 nt, Homo sapiens
aagugccgccaucuuuugagugu

SEQ ID NO: 527 - RNA, 22 nt, Homo sapiens
cuuaucagauuguauuguaauu

SEQ ID NO: 528 - RNA, 22 nt, Homo sapiens
uggguuccuggcaugcugauuu

SEQ ID NO: 529 - RNA, 22 nt, Homo sapiens
guagauucuccuucuaugagua

SEQ ID NO: 530 - RNA, 22 nt, Homo sapiens
agagguugcccuuggugaauuc

SEQ ID NO: 531 - RNA, 22 nt, Homo sapiens
cugggagguggauguuuacuuc

SEQ ID NO: 532 - RNA, 22 nt, Homo sapiens
aacgccauuaucacacuaaaua

SEQ ID NO: 533 - RNA, 22 nt, Homo sapiens
uucacauugugcuacugucugc

SEQ ID NO: 534 - RNA, 22 nt, Homo sapiens
accguggcuuucgauuguuacu

SEQ ID NO: 535 - RNA, 22 nt, Homo sapiens
uauguaacaugguccacuaacu

SEQ ID NO: 536 - RNA, 22 nt, Homo sapiens
aaaguucugagacacuccgacu

SEQ ID NO: 537 - RNA, 21 nt, Homo sapiens
gugcauuguaguugcauugca

SEQ ID NO: 538 - RNA, 22 nt, Homo sapiens
caauguuuccacagugcaucac

SEQ ID NO: 539 - RNA, 23 nt, Homo sapiens
agguugggaucgguugcaaugcu

SEQ ID NO: 540 - RNA, 22 nt, Homo sapiens
ggguggggauuuguugcauuac

SEQ ID NO: 541 - RNA, 22 nt, Homo sapiens
acugcugagcuagcacuucccg

SEQ ID NO: 542 - RNA, 22 nt, Homo sapiens
aaucaugugcagugccaauaug

SEQ ID NO: 543 - RNA, 22 nt, Homo sapiens
caagcucgcuucuaugggucug

SEQ ID NO: 544 - RNA, 22 nt, Homo sapiens
caagcuuguaucuauagguaug

SEQ ID NO: 545 - RNA, 22 nt, Homo sapiens
caguuaucacagugcugaugcu

SEQ ID NO: 546 - RNA, 22 nt, Homo sapiens
gcuauuucacgacaccaggguu

SEQ ID NO: 547 - RNA, 22 nt, Homo sapiens
caucuuccaguacaguguugga

SEQ ID NO: 548 - RNA, 22 nt, Homo sapiens
ggugcagugcugcaucucuggu

SEQ ID NO: 549 - RNA, 21 nt, Homo sapiens
aggggugcuaucugugauuga

SEQ ID NO: 550 - RNA, 22 nt, Homo sapiens
ggauaucaucauauacuguaag

SEQ ID NO: 551 - RNA, 22 nt, Homo sapiens
ggauuccuggaaauacuguucu

SEQ ID NO: 552 - RNA, 20 nt, Homo sapiens
ggggagcuguggaagcagua

SEQ ID NO: 553 - RNA, 25 nt, Homo sapiens
cuagugagggacagaaccaggauuc

SEQ ID NO: 554 - RNA, 23 nt, Homo sapiens
gcagcagagaauaggacuacguc

SEQ ID NO: 555 - RNA, 21 nt, Homo sapiens
gucagcggaggaaaagaaacu

SEQ ID NO: 556 - RNA, 20 nt, Homo sapiens
agagucuugugaugucuugc

SEQ ID NO: 557 - RNA, 22 nt, Homo sapiens
uacugcagacguggcaaucaug

SEQ ID NO: 558 - RNA, 21 nt, Homo sapiens
gaacggcuucauacaggaguu

SEQ ID NO: 559 - RNA, 22 nt, Homo sapiens
cuccuauaugaugccuuucuuc

SEQ ID NO: 560 - RNA, 22 nt, Homo sapiens
ucacaagucaggcucuugggac

SEQ ID NO: 561 - RNA, 22 nt, Homo sapiens
auguagggcuaaaagccauggg

SEQ ID NO: 562 - RNA, 22 nt, Homo sapiens
aaguucuguuauacacucaggc

SEQ ID NO: 563 - RNA, 20 nt, Homo sapiens
acugccccaggugcugcugg

SEQ ID NO: 564 - RNA, 22 nt, Homo sapiens
gcuacuucacaacaccagggcc

SEQ ID NO: 565 - RNA, 22 nt, Homo sapiens
ccucugaaauucaguucuucag

SEQ ID NO: 566 - RNA, 21 nt, Homo sapiens
agggagggacgggggcugugc

SEQ ID NO: 567 - RNA, 24 nt, Homo sapiens
gcugguuucauauggugguuuaga

SEQ ID NO: 568 - RNA, 22 nt, Homo sapiens
cugguuucacaugguggcuuag

SEQ ID NO: 569 - RNA, 23 nt, Homo sapiens
ucaaaugcucagacuccuguggu

SEQ ID NO: 570 - RNA, 22 nt, Homo sapiens
acggauguuugagcaugugcua

SEQ ID NO: 571 - RNA, 23 nt, Homo sapiens
aaaagugcuuacagugcagguag

SEQ ID NO: 572 - RNA, 22 nt, Homo sapiens
cugcaauguaagcacuucuuac

SEQ ID NO: 573 - RNA, 22 nt, Homo sapiens
ccaauauuacugugcugcuuua

SEQ ID NO: 574 - RNA, 22 nt, Homo sapiens
cugcgcaagcuacugccuugcu

SEQ ID NO: 575 - RNA, 22 nt, Homo sapiens
cgaaucauuauuugcugcucua

SEQ ID NO: 576 - RNA, 22 nt, Homo sapiens
agagcuuagcugauuggugaac

SEQ ID NO: 577 - RNA, 22 nt, Homo sapiens
ugugcgcagggagaccucuccc

SEQ ID NO: 578 - RNA, 22 nt, Homo sapiens
ugucuacuacuggagacacugg

SEQ ID NO: 579 - RNA, 23 nt, Homo sapiens
ccaguuaccgcuuccgcuaccgc

SEQ ID NO: 580 - RNA, 22 nt, Homo sapiens
acaguagagggaggaaucgcag

SEQ ID NO: 581 - RNA, 22 nt, Homo sapiens
auccgcgcucugacucucugcc

SEQ ID NO: 582 - RNA, 22 nt, Homo sapiens
ugcccuuaaaggugaacccagu

SEQ ID NO: 583 - RNA, 24 nt, Homo sapiens
uggggagcugaggcucugggggug

SEQ ID NO: 584 - RNA, 23 nt, Homo sapiens
cacccggcugugugcacaugugc

SEQ ID NO: 585 - RNA, 23 nt, Homo sapiens
ugagcgccucgacgacagagccg

SEQ ID NO: 586 - RNA, 22 nt, Homo sapiens
uuuuucauuauugcuccugacc

SEQ ID NO: 587 - RNA, 22 nt, Homo sapiens
gcugacuccuaguccagggcuc

SEQ ID NO: 588 - RNA, 22 nt, Homo sapiens
ucuucucuguuuuggccaugug

SEQ ID NO: 589 - RNA, 21 nt, Homo sapiens
cugacuguugccguccuccag

SEQ ID NO: 590 - RNA, 22 nt, Homo sapiens
aaauuauuguacaucggaugag

SEQ ID NO: 591 - RNA, 24 nt, Homo sapiens
agcagaagcagggagguucuccca

SEQ ID NO: 592 - RNA, 22 nt, Homo sapiens
ugcaacgaaccugagccacuga

SEQ ID NO: 593 - RNA, 23 nt, Homo sapiens
cgggucggaguuagcucaagcgg

SEQ ID NO: 594 - RNA, 21 nt, Homo sapiens
cgcgggugcuuacugacccuu

SEQ ID NO: 595 - RNA, 21 nt, Homo sapiens
cacuguguccuuucugcguag

SEQ ID NO: 596 - RNA, 22 nt, Homo sapiens
caagcucgugucuguggguccg

SEQ ID NO: 597 - RNA, 22 nt, Homo sapiens
cguguucacagcggaccuugau

SEQ ID NO: 598 - RNA, 24 nt, Homo sapiens
ucccugagacccuuuaaccuguga

SEQ ID NO: 599 - RNA, 22 nt, Homo sapiens
acaggugagguucuugggagcc

SEQ ID NO: 600 - RNA, 25 nt, Homo sapiens
aaaggauucugcugucggucccacu

SEQ ID NO: 601 - RNA, 22 nt, Homo sapiens
uggugggcacagaaucuggacu

SEQ ID NO: 602 - RNA, 21 nt, Homo sapiens
uuaauaucggacaaccauugu

SEQ ID NO: 603 - RNA, 22 nt, Homo sapiens
uauaccucaguuuuaucaggug

SEQ ID NO: 604 - RNA, 21 nt, Homo sapiens
ccuggaaacacugagguugug

SEQ ID NO: 605 - RNA, 22 nt, Homo sapiens
uggauuucuuugugaaucacca

SEQ ID NO: 606 - RNA, 21 nt, Homo sapiens
ccaccaccgugucugacacuu

SEQ ID NO: 607 - RNA, 22 nt, Homo sapiens
uuuugcaauauguuccugaaua

SEQ ID NO: 608 - RNA, 22 nt, Homo sapiens
uugggaucauuuugcauccaua

SEQ ID NO: 609 - RNA, 21 nt, Homo sapiens
uacuuggaaaggcaucaguug

SEQ ID NO: 610 - RNA, 22 nt, Homo sapiens
ugcaacuuaccugagucauuga

SEQ ID NO: 611 - RNA, 22 nt, Homo sapiens
acacagggcuguugugaagacu

SEQ ID NO: 612 - RNA, 21 nt, Homo sapiens
uacucaaaaagcugucaguca

SEQ ID NO: 613 - RNA, 22 nt, Homo sapiens
gacugacaccucuuugggugaa

SEQ ID NO: 614 - RNA, 22 nt, Homo sapiens
cacuggcuccuuucuggguaga

SEQ ID NO: 615 - RNA, 22 nt, Homo sapiens
uagguaguuuccuguuguuggg

SEQ ID NO: 616 - RNA, 21 nt, Homo sapiens
ucacagugaaccggucucuuu

SEQ ID NO: 617 - RNA, 23 nt, Homo sapiens
uaaggugcaucuagugcaguuag

SEQ ID NO: 618 - RNA, 23 nt, Homo sapiens
uacccuguagaaccgaauuugug

SEQ ID NO: 619 - RNA, 22 nt, Homo sapiens
uaaucucagcuggcaacuguga

SEQ ID NO: 620 - RNA, 22 nt, Homo sapiens
ugagguaguaguuugugcuguu

SEQ ID NO: 621 - RNA, 22 nt, Homo sapiens
uggaauguaaagaaguauguau

SEQ ID NO: 622 - RNA, 22 nt, Homo sapiens
uguaaacauccuugacuggaag

SEQ ID NO: 623 - RNA, 22 nt, Homo sapiens
uggugguuuacaaaguaauuca

SEQ ID NO: 624 - RNA, 21 nt, Homo sapiens
cacauuacacggucgaccucu

SEQ ID NO: 625 - RNA, 22 nt, Homo sapiens
ucguaccgugaguaauaaugcg

SEQ ID NO: 626 - RNA, 22 nt, Homo sapiens
cugaagcucagagggcucugau

SEQ ID NO: 627 - RNA, 22 nt, Homo sapiens
ucucugggccugugucuuaggc

SEQ ID NO: 628 - RNA, 22 nt, Homo sapiens
auaaagcuagauaaccgaaagu

SEQ ID NO: 629 - RNA, 22 nt, Homo sapiens
uauagggauuggagccguggcg

SEQ ID NO: 630 - RNA, 22 nt, Homo sapiens
cuagguauggucccagggaucc

SEQ ID NO: 631 - RNA, 21 nt, Homo sapiens
uaccacaggguagaaccacgg

SEQ ID NO: 632 - RNA, 21 nt, Homo sapiens
uacugcagacaguggcaauca

SEQ ID NO: 633 - RNA, 22 nt, Homo sapiens
ugauugguacgucuguggguag

SEQ ID NO: 634 - RNA, 22 nt, Homo sapiens
aaaaguaauugugguuuuugcc

SEQ ID NO: 635 - RNA, 22 nt, Homo sapiens
uauguaacacgguccacuaacc

SEQ ID NO: 636 - RNA, 22 nt, Homo sapiens
uaugucugcugaccaucaccuu

SEQ ID NO: 637 - RNA, 23 nt, Homo sapiens
ucggggaucaucaugucacgaga

SEQ ID NO: 638 - RNA, 22 nt, Homo sapiens
uacucaggagaguggcaaucac

SEQ ID NO: 639 - RNA, 21 nt, Homo sapiens
acuggacuuggagucagaagg

SEQ ID NO: 640 - RNA, 21 nt, Homo sapiens
gcaguccaugggcauauacac

SEQ ID NO: 641 - RNA, 22 nt, Homo sapiens
uggagugugacaaugguguuug

SEQ ID NO: 642 - RNA, 22 nt, Homo sapiens
uuugguccccuucaaccagcug

SEQ ID NO: 643 - RNA, 22 nt, Homo sapiens
uuugguccccuucaaccagcua

SEQ ID NO: 644 - RNA, 21 nt, Homo sapiens
cauaaaguagaaagcacuacu

SEQ ID NO: 645 - RNA, 21 nt, Homo sapiens
ugagaugaagcacuguagcuc

SEQ ID NO: 646 - RNA, 22 nt, Homo sapiens
aacuggccuacaaagucccagu

SEQ ID NO: 647 - RNA, 22 nt, Homo sapiens
uaauacugccugguaaugauga

SEQ ID NO: 648 - RNA, 22 nt, Homo sapiens
uccagcaucagugauuuuguug

SEQ ID NO: 649 - RNA, 21 nt, Homo sapiens
uacaguacugugauaacugaa

SEQ ID NO: 650 - RNA, 21 nt, Homo sapiens
cuagacugaagcuccuugagg

SEQ ID NO: 651 - RNA, 23 nt, Homo sapiens
ucuggcuccgugucuucacuccc

SEQ ID NO: 652 - RNA, 23 nt, Homo sapiens
ucccuguccuccaggagcucacg

SEQ ID NO: 653 - RNA, 22 nt, Homo sapiens
uuauaaagcaaugagacugauu

SEQ ID NO: 654 - RNA, 22 nt, Homo sapiens
uccgucucaguuacuuuauagc

SEQ ID NO: 655 - RNA, 23 nt, Homo sapiens
ucucacacagaaaucgcacccgu

SEQ ID NO: 656 - RNA, 23 nt, Homo sapiens
uauggcuuuucauuccuauguga

SEQ ID NO: 657 - RNA, 22 nt, Homo sapiens
gugugcggaaaugcuucugcua

SEQ ID NO: 658 - RNA, 21 nt, Homo sapiens
ugauauguuugauauuggguu

SEQ ID NO: 659 - RNA, 21 nt, Homo sapiens
aagguuacuuguuaguucagg

SEQ ID NO: 660 - RNA, 22 nt, Homo sapiens
auucugcauuuuuagcaaguuc

SEQ ID NO: 661 - RNA, 22 nt, Homo sapiens
ucaguaaauguuuauuagauga

SEQ ID NO: 662 - RNA, 22 nt, Homo sapiens
ucagcaaacauuuauugugugc

SEQ ID NO: 663 - RNA, 22 nt, Homo sapiens
cugcccuggcccgagggaccga

SEQ ID NO: 664 - RNA, 22 nt, Homo sapiens
uauggcacugguagaauucacu

SEQ ID NO: 665 - RNA, 22 nt, Homo sapiens
gugaauuaccgaagggccauaa

SEQ ID NO: 666 - RNA, 22 nt, Homo sapiens
uggagagaaaggcaguuccuga

SEQ ID NO: 667 - RNA, 22 nt, Homo sapiens
cugccaauuccauaggucacag

SEQ ID NO: 668 - RNA, 22 nt, Homo sapiens
gguccagaggggagauagguuc

SEQ ID NO: 669 - RNA, 22 nt, Homo sapiens
caucuuacugggcagcauugga

SEQ ID NO: 670 - RNA, 22 nt, Homo sapiens
gccugcugggguggaaccuggu

SEQ ID NO: 671 - RNA, 21 nt, Homo sapiens
agcuacaucuggcuacugggu

SEQ ID NO: 672 - RNA, 22 nt, Homo sapiens
aaaagcuggguugagagggcga

SEQ ID NO: 673 - RNA, 23 nt, Homo sapiens
guccaguuuucccaggaaucccu

SEQ ID NO: 674 - RNA, 21 nt, Homo sapiens
aggcaagaugcuggcauagcu

SEQ ID NO: 675 - RNA, 22 nt, Homo sapiens
ugggucuuugcgggcgagauga

SEQ ID NO: 676 - RNA, 22 nt, Homo sapiens
ugagguaguaguuuguacaguu

SEQ ID NO: 677 - RNA, 22 nt, Homo sapiens
agagguaguagguugcauaguu

SEQ ID NO: 678 - RNA, 23 nt, Homo sapiens
agcugguguugugaaucaggccg

SEQ ID NO: 679 - RNA, 22 nt, Homo sapiens
caaagaauucuccuuuugggcu

SEQ ID NO: 680 - RNA, 22 nt, Homo sapiens
cgucuuacccagcaguguuugg

SEQ ID NO: 681 - RNA, 22 nt, Homo sapiens
cuccuacauauuagcauuaaca

SEQ ID NO: 682 - RNA, 22 nt, Homo sapiens
caaauucguaucuaggggaaua

SEQ ID NO: 683 - RNA, 22 nt, Homo sapiens
ucuacagugcacgugucuccag

SEQ ID NO: 684 - RNA, 22 nt, Homo sapiens
auaagacgaacaaaagguuugu

SEQ ID NO: 685 - RNA, 21 nt, Homo sapiens
guguugaaacaaucucuacug

SEQ ID NO: 686 - RNA, 22 nt, Homo sapiens
ugccugucuacacuugcugugc

SEQ ID NO: 687 - RNA, 22 nt, Homo sapiens
caugguucugucaagcaccgcg

SEQ ID NO: 688 - RNA, 22 nt, Homo sapiens
ugucaguuugucaaauacccca

SEQ ID NO: 689 - RNA, 22 nt, Homo sapiens
uccauuacacuacccugccucu

SEQ ID NO: 690 - RNA, 22 nt, Homo sapiens
acuggacuuagggucagaaggc

SEQ ID NO: 691 - RNA, 22 nt, Homo sapiens
aagcccuuaccccaaaaaguau

SEQ ID NO: 692 - RNA, 23 nt, Homo sapiens
caacggaaucccaaaagcagcug

SEQ ID NO: 693 - RNA, 23 nt, Homo sapiens
uaauacugccggguaaugaugga

SEQ ID NO: 694 - RNA, 22 nt, Homo sapiens
aguucuucaguggcaagcuuua

SEQ ID NO: 695 - RNA, 22 nt, Homo sapiens
aucgggaaugucguguccgccc

SEQ ID NO: 696 - RNA, 22 nt, Homo sapiens
uuuugcgauguguuccuaauau

SEQ ID NO: 697 - RNA, 22 nt, Homo sapiens
acaguagucugcacauugguua

SEQ ID NO: 698 - RNA, 22 nt, Homo sapiens
cuuucagucagauguuugcugc

SEQ ID NO: 699 - RNA, 22 nt, Homo sapiens
acagcaggcacagacaggcagu

SEQ ID NO: 700 - RNA, 21 nt, Homo sapiens
cuauacaaucuacugucuuuc

SEQ ID NO: 701 - RNA, 21 nt, Homo sapiens
caaaacgugaggcgcugcuau

SEQ ID NO: 702 - RNA, 22 nt, Homo sapiens
ugcccuaaaugccccuucuggc

SEQ ID NO: 703 - RNA, 22 nt, Homo sapiens
acuguaguaugggcacuuccag

SEQ ID NO: 704 - RNA, 22 nt, Homo sapiens
caggucgucuugcagggcuucu

SEQ ID NO: 705 - RNA, 22 nt, Homo sapiens
ggagacgcggcccuguuggagu

SEQ ID NO: 706 - RNA, 22 nt, Homo sapiens
caacaaaucccagucuaccuaa

SEQ ID NO: 707 - RNA, 22 nt, Homo sapiens
acagauucgauucuaggggaau

SEQ ID NO: 708 - RNA, 22 nt, Homo sapiens
caaucagcaaguauacugcccu

SEQ ID NO: 709 - RNA, 22 nt, Homo sapiens
accacugaccguugacuguacc

SEQ ID NO: 710 - RNA, 23 nt, Homo sapiens
agguuguccguggugaguucgca

SEQ ID NO: 711 - RNA, 21 nt, Homo sapiens
caucccuugcaugguggaggg

SEQ ID NO: 712 - RNA, 21 nt, Homo sapiens
uccgguucucagggcuccacc

SEQ ID NO: 713 - RNA, 23 nt, Homo sapiens
uagugcaauauugcuuauagggu

SEQ ID NO: 714 - RNA, 22 nt, Homo sapiens
ugcggggcuagggcuaacagca

SEQ ID NO: 715 - RNA, 22 nt, Homo sapiens
cuguugccacuaaccucaaccu

SEQ ID NO: 716 - RNA, 22 nt, Homo sapiens
aaaucucugcaggcaaauguga

SEQ ID NO: 717 - RNA, 23 nt, Homo sapiens
ugagguugguguacuguguguga

SEQ ID NO: 718 - RNA, 20 nt, Homo sapiens
cggcucugggucugugggga

SEQ ID NO: 719 - RNA, 22 nt, Homo sapiens
aacuguuugcagaggaaacuga

SEQ ID NO: 720 - RNA, 22 nt, Homo sapiens
cucaucugcaaagaaguaagug

SEQ ID NO: 721 - RNA, 23 nt, Homo sapiens
agguuacccgagcaacuuugcau

SEQ ID NO: 722 - RNA, 22 nt, Homo sapiens
gaauguugcucggugaaccccu

SEQ ID NO: 723 - RNA, 22 nt, Homo sapiens
aaccaucgaccguugaguggac

SEQ ID NO: 724 - RNA, 24 nt, Homo sapiens
uuuggcaaugguagaacucacacu

SEQ ID NO: 725 - RNA, 22 nt, Homo sapiens
cggcaacaagaaacugccugag

SEQ ID NO: 726 - RNA, 23 nt, Homo sapiens
uacugcaucaggaacugauugga

SEQ ID NO: 727 - RNA, 22 nt, Homo sapiens
aagacgggaggaaagaagggag

SEQ ID NO: 728 - RNA, 21 nt, Homo sapiens
ucacuccucuccucccgucuu

SEQ ID NO: 729 - RNA, 23 nt, Homo sapiens
ugaggggcagagagcgagacuuu

SEQ ID NO: 730 - RNA, 23 nt, Homo sapiens
aaggagcuuacaaucuagcuggg

SEQ ID NO: 731 - RNA, 22 nt, Homo sapiens
caacuagacugugagcuucuag

SEQ ID NO: 732 - RNA, 22 nt, Homo sapiens
agggacgggacgcggugcagug

SEQ ID NO: 733 - RNA, 22 nt, Homo sapiens
gaugagcucauuguaauaugag

SEQ ID NO: 734 - RNA, 22 nt, Homo sapiens
auauuaccauuagcucaucuuu

SEQ ID NO: 735 - RNA, 22 nt, Homo sapiens
gaaaucaagcgugggugagacc

SEQ ID NO: 736 - RNA, 22 nt, Homo sapiens
cgaaaacagcaauuaccuuugc

SEQ ID NO: 737 - RNA, 22 nt, Homo sapiens
cacgcucaugcacacacccaca

SEQ ID NO: 738 - RNA, 22 nt, Homo sapiens
auucuaauuucuccacgucuuu

SEQ ID NO: 739 - RNA, 22 nt, Homo sapiens
aagauguggaaaaauuggaauc

SEQ ID NO: 740 - RNA, 21 nt, Homo sapiens
aauggcgccacuaggguugug

SEQ ID NO: 741 - RNA, 22 nt, Homo sapiens
ggggguccccggugcucggauc

SEQ ID NO: 742 - RNA, 23 nt, Homo sapiens
uauucagauuagugccagucaug

SEQ ID NO: 743 - RNA, 22 nt, Homo sapiens
ccucccacacccaaggcuugca

SEQ ID NO: 744 - RNA, 21 nt, Homo sapiens
gcaggaacuugugagucuccu

SEQ ID NO: 745 - RNA, 21 nt, Homo sapiens
uugaaaggcuauuucuugguc

SEQ ID NO: 746 - RNA, 22 nt, Homo sapiens
gugacaucacauauacggcagc

SEQ ID NO: 747 - RNA, 22 nt, Homo sapiens
cuuaugcaagauucccuucuac

SEQ ID NO: 748 - RNA, 22 nt, Homo sapiens
ugcccuguggacucaguucugg

SEQ ID NO: 749 - RNA, 22 nt, Homo sapiens
uuccuaugcauauacuucuuug

SEQ ID NO: 750 - RNA, 20 nt, Homo sapiens
agagguauagggcaugggaa

SEQ ID NO: 751 - RNA, 22 nt, Homo sapiens
ugaaggucuacugugugccagg

SEQ ID NO: 752 - RNA, 22 nt, Homo sapiens
ugaaacauacacgggaaaccuc

SEQ ID NO: 753 - RNA, 22 nt, Homo sapiens
cgggguuuugagggcgagauga

SEQ ID NO: 754 - RNA, 22 nt, Homo sapiens
aacuggcccucaaagucccgcu

SEQ ID NO: 755 - RNA, 22 nt, Homo sapiens
caaaccacacugugguguuaga

SEQ ID NO: 756 - RNA, 22 nt, Homo sapiens
gagugccuucuuuuggagcguu

SEQ ID NO: 757 - RNA, 22 nt, Homo sapiens
aagugccuccuuuuagaguguu

SEQ ID NO: 758 - RNA, 22 nt, Homo sapiens
cucuagagggaagcgcuuucug

SEQ ID NO: 759 - RNA, 22 nt, Homo sapiens
aggcagcgggguguaguggaua

SEQ ID NO: 760 - RNA, 22 nt, Homo sapiens
gugaacgggcgccaucccgagg

SEQ ID NO: 761 - RNA, 22 nt, Homo sapiens
aaacauucgcggugcacuucuu

SEQ ID NO: 762 - RNA, 22 nt, Homo sapiens
acggguuaggcucuugggagcu

SEQ ID NO: 763 - RNA, 22 nt, Homo sapiens
ccaguggggcugcuguuaucug

SEQ ID NO: 764 - RNA, 22 nt, Homo sapiens
ccgcacuguggguacuugcugc

SEQ ID NO: 765 - RNA, 23 nt, Homo sapiens
acuuaaacguggauguacuugcu

SEQ ID NO: 766 - RNA, 23 nt, Homo sapiens
cucuugagggaagcacuuucugu

SEQ ID NO: 767 - RNA, 22 nt, Homo sapiens
gaaagugcuuccuuuuagaggc

SEQ ID NO: 768 - RNA, 22 nt, Homo sapiens
aaagugcauccuuuuagagguu

SEQ ID NO: 769 - RNA, 22 nt, Homo sapiens
gaaggcgcuucccuuuagagcg

SEQ ID NO: 770 - RNA, 23 nt, Homo sapiens
gaacgcgcuucccuauagagggu

SEQ ID NO: 771 - RNA, 22 nt, Homo sapiens
cucuagagggaagcacuuucuc

SEQ ID NO: 772 - RNA, 21 nt, Homo sapiens
gaaagcgcuucucuuuagagg

SEQ ID NO: 773 - RNA, 22 nt, Homo sapiens
cucuagagggaagcacuuucug

SEQ ID NO: 774 - RNA, 22 nt, Homo sapiens
agaauuguggcuggacaucugu

SEQ ID NO: 775 - RNA, 22 nt, Homo sapiens
cuuagcagguuguauuaucauu

SEQ ID NO: 776 - RNA, 23 nt, Homo sapiens
cagugcaaugauauugucaaagc

SEQ ID NO: 777 - RNA, 20 nt, Homo sapiens
cuacaaagggaagcccuuuc

SEQ ID NO: 778 - RNA, 21 nt, Homo sapiens
aaagcgcuucccuucagagug

SEQ ID NO: 779 - RNA, 20 nt, Homo sapiens
cugcaaagggaagcccuuuc

SEQ ID NO: 780 - RNA, 22 nt, Homo sapiens
gaaagcgcuucccuuugcugga

SEQ ID NO: 781 - RNA, 23 nt, Homo sapiens
uucauuugguauaaaccgcgauu

SEQ ID NO: 782 - RNA, 22 nt, Homo sapiens
uaacugguugaacaacugaacc

SEQ ID NO: 783 - RNA, 22 nt, Homo sapiens
aaagugcuuccuuuuagagggu

SEQ ID NO: 784 - RNA, 23 nt, Homo sapiens
caaagcgcuucucuuuagagugu

SEQ ID NO: 785 - RNA, 22 nt, Homo sapiens
aucgugcaucccuuuagagugu

SEQ ID NO: 786 - RNA, 22 nt, Homo sapiens
caaagugccucccuuuagagug

SEQ ID NO: 787 - RNA, 22 nt, Homo sapiens
cuauacaaccuacugccuuccc

SEQ ID NO: 788 - RNA, 22 nt, Homo sapiens
uagaguuacacccugggaguua

SEQ ID NO: 789 - RNA, 22 nt, Homo sapiens
ugagguaggagguuguauaguu

SEQ ID NO: 790 - RNA, 22 nt, Homo sapiens
cuauacggccuccuagcuuucc

SEQ ID NO: 791 - RNA, 22 nt, Homo sapiens
aaaaguaauugugguuuuggcc

SEQ ID NO: 792 - RNA, 22 nt, Homo sapiens
ugagaaccacgucugcucugag

SEQ ID NO: 793 - RNA, 23 nt, Homo sapiens
agugccugagggaguaagagccc

SEQ ID NO: 794 - RNA, 19 nt, Homo sapiens
ugucucugcugggguuucu

SEQ ID NO: 795 - RNA, 22 nt, Homo sapiens
aaaaguaauugcgaguuuuacc

SEQ ID NO: 796 - RNA, 22 nt, Homo sapiens
aaaaugguucccuuuagagugu

SEQ ID NO: 797 - RNA, 22 nt, Homo sapiens
agucauuggaggguuugagcag

SEQ ID NO: 798 - RNA, 22 nt, Homo sapiens
aaagugcauccuuuuagagugu

SEQ ID NO: 799 - RNA, 23 nt, Homo sapiens
uucucgaggaaagaagcacuuuc

SEQ ID NO: 800 - RNA, 22 nt, Homo sapiens
cuauacaaucuauugccuuccc

SEQ ID NO: 801 - RNA, 22 nt, Homo sapiens
cuauacagucuacugucuuucc

SEQ ID NO: 802 - RNA, 22 nt, Homo sapiens
caggccauauugugcugccuca

SEQ ID NO: 803 - RNA, 22 nt, Homo sapiens
ccaguauuaacugugcugcuga

SEQ ID NO: 804 - RNA, 22 nt, Homo sapiens
acugcagugaaggcacuuguag

SEQ ID NO: 805 - RNA, 23 nt, Homo sapiens
acugcccuaagugcuccuucugg

SEQ ID NO: 806 - RNA, 22 nt, Homo sapiens
aguuuugcauaguugcacuaca

SEQ ID NO: 807 - RNA, 23 nt, Homo sapiens
aguuuugcagguuugcauccagc

SEQ ID NO: 808 - RNA, 22 nt, Homo sapiens
aguuuugcagguuugcauuuca

SEQ ID NO: 809 - RNA, 22 nt, Homo sapiens
aacaucacagcaagucugugcu

SEQ ID NO: 810 - RNA, 23 nt, Homo sapiens
uaauccuugcuaccugggugaga

SEQ ID NO: 811 - RNA, 22 nt, Homo sapiens
aaaaguaauugcgguuuuugcc

SEQ ID NO: 812 - RNA, 21 nt, Homo sapiens
cacaagguauugguauuaccu

SEQ ID NO: 813 - RNA, 21 nt, Homo sapiens
agggggaaaguucuauagucc

SEQ ID NO: 814 - RNA, 22 nt, Homo sapiens
gacuauagaacuuucccccuca

SEQ ID NO: 815 - RNA, 22 nt, Homo sapiens
augcugacauauuuacuagagg

SEQ ID NO: 816 - RNA, 21 nt, Homo sapiens
ucuaguaagaguggcagucga

SEQ ID NO: 817 - RNA, 22 nt, Homo sapiens
aaugcacccgggcaaggauucu

SEQ ID NO: 818 - RNA, 21 nt, Homo sapiens
uggguuuacguugggagaacu

SEQ ID NO: 819 - RNA, 22 nt, Homo sapiens
acugcauuaugagcacuuaaag

SEQ ID NO: 820 - RNA, 21 nt, Homo sapiens
caacaccagucgaugggcugu

SEQ ID NO: 821 - RNA, 22 nt, Homo sapiens
gggguuccuggggaugggauuu

SEQ ID NO: 822 - RNA, 22 nt, Homo sapiens
ugccuacugagcugauaucagu

SEQ ID NO: 823 - RNA, 22 nt, Homo sapiens
ugccuacugagcugaaacacag

SEQ ID NO: 824 - RNA, 21 nt, Homo sapiens
aggcggagacuugggcaauug

SEQ ID NO: 825 - RNA, 22 nt, Homo sapiens
ccuauucuugguuacuugcacg

SEQ ID NO: 826 - RNA, 22 nt, Homo sapiens
ccuguucuccauuacuuggcuc

SEQ ID NO: 827 - RNA, 22 nt, Homo sapiens
agggcuuagcugcuugugagca

SEQ ID NO: 828 - RNA, 22 nt, Homo sapiens
cacuagauugugagcuccugga

SEQ ID NO: 829 - RNA, 22 nt, Homo sapiens
acugauuucuuuugguguucag

SEQ ID NO: 830 - RNA, 22 nt, Homo sapiens
ugagguaguagguugugugguu

SEQ ID NO: 831 - RNA, 23 nt, Homo sapiens
uuaaugcuaaucgugauaggggu

SEQ ID NO: 832 - RNA, 23 nt, Homo sapiens
agcucggucugaggccccucagu

SEQ ID NO: 833 - RNA, 22 nt, Homo sapiens
cugguacaggccugggggacag

SEQ ID NO: 834 - RNA, 21 nt, Homo sapiens
ucgaggagcucacagucuagu

SEQ ID NO: 835 - RNA, 21 nt, Homo sapiens
uggaggagaaggaaggugaug

SEQ ID NO: 836 - RNA, 22 nt, Homo sapiens
aacaauauccuggugcugagug

SEQ ID NO: 837 - RNA, 20 nt, Homo sapiens
auggagauagauauagaaau

SEQ ID NO: 838 - RNA, 21 nt, Homo sapiens
uagauaaaauauugguaccug

SEQ ID NO: 839 - RNA, 20 nt, Homo sapiens
uacaguauagaugauguacu

SEQ ID NO: 840 - RNA, 21 nt, Homo sapiens
uaauuuuauguauaagcuagu

SEQ ID NO: 841 - RNA, 22 nt, Homo sapiens
gcugcgcuuggauuucgucccc

SEQ ID NO: 842 - RNA, 20 nt, Homo sapiens
accaggaggcugaggccccu

SEQ ID NO: 843 - RNA, 22 nt, Homo sapiens
aggugguccguggcgcguucgc

SEQ ID NO: 844 - RNA, 22 nt, Homo sapiens
ggcuacaacacaggacccgggc

SEQ ID NO: 845 - RNA, 22 nt, Homo sapiens
aaagugcuucucuuuggugggu

SEQ ID NO: 846 - RNA, 21 nt, Homo sapiens
ccccaccuccucucuccucag

SEQ ID NO: 847 - RNA, 21 nt, Homo sapiens
uccucuucucccuccucccag

SEQ ID NO: 848 - RNA, 22 nt, Homo sapiens
uucucaaggaggugucguuuau

SEQ ID NO: 849 - RNA, 22 nt, Homo sapiens
uucacaaggaggugucauuuau

SEQ ID NO: 850 - RNA, 26 nt, Homo sapiens
gugagggcaugcaggccuggaugggg

SEQ ID NO: 851 - RNA, 22 nt, Homo sapiens
ccucuuccccuugucucuccag

SEQ ID NO: 852 - RNA, 20 nt, Homo sapiens
gugucugggcggacagcugc

SEQ ID NO: 853 - RNA, 21 nt, Homo sapiens
gugggcgggggcaggugugug

SEQ ID NO: 854 - RNA, 22 nt, Homo sapiens
guggguacggcccagugggggg

SEQ ID NO: 855 - RNA, 21 nt, Homo sapiens
uccuucugcuccgucccccag

SEQ ID NO: 856 - RNA, 22 nt, Homo sapiens
ugagccccugugccgcccccag

SEQ ID NO: 857 - RNA, 20 nt, Homo sapiens
ugagcccuguccucccgcag

SEQ ID NO: 858 - RNA, 20 nt, Homo sapiens
cgugccacccuuuuccccag

SEQ ID NO: 859 - RNA, 21 nt, Homo sapiens
ugcaggaccaagaugagcccu

SEQ ID NO: 860 - RNA, 21 nt, Homo sapiens
caaagguauuugugguuuuug

SEQ ID NO: 861 - RNA, 21 nt, Homo sapiens
aagcauucuuucauugguugg

SEQ ID NO: 862 - RNA, 21 nt, Homo sapiens
uugcucacuguucuucccuag

SEQ ID NO: 863 - RNA, 20 nt, Homo sapiens
ucugcaggguuugcuuugag

SEQ ID NO: 864 - RNA, 22 nt, Homo sapiens
cuuggcaccuagcaagcacuca

SEQ ID NO: 865 - RNA, 24 nt, Homo sapiens
agccugauuaaacacaugcucuga

SEQ ID NO: 866 - RNA, 25 nt, Homo sapiens
gggcgacaaagcaagacucuuucuu

SEQ ID NO: 867 - RNA, 22 nt, Homo sapiens
aaaaguaauugcggucuuuggu

SEQ ID NO: 868 - RNA, 22 nt, Homo sapiens
augguacccuggcauacugagu

SEQ ID NO: 869 - RNA, 22 nt, Homo sapiens
ugugagguuggcauuguugucu

SEQ ID NO: 870 - RNA, 22 nt, Homo sapiens
ucaaaacugaggggcauuuucu

SEQ ID NO: 871 - RNA, 19 nt, Homo sapiens
gaugaugcugcugaugcug

SEQ ID NO: 872 - RNA, 22 nt, Homo sapiens
cuggacugagccgugcuacugg

SEQ ID NO: 873 - RNA, 22 nt, Homo sapiens
caggauguggucaaguguuguu

SEQ ID NO: 874 - RNA, 26 nt, Homo sapiens
aaguaguugguuuguaugagaugguu

SEQ ID NO: 875 - RNA, 22 nt, Homo sapiens
uuuagagacggggucuugcucu

SEQ ID NO: 876 - RNA, 21 nt, Homo sapiens
auauaugaugacuuagcuuuu

SEQ ID NO: 877 - RNA, 17 nt, Homo sapiens
uaauugcuuccauguuu

SEQ ID NO: 878 - RNA, 22 nt, Homo sapiens
uagcaaaaacugcaguuacuuu

SEQ ID NO: 879 - RNA, 23 nt, Homo sapiens
caagucuuauuugagcaccuguu

SEQ ID NO: 880 - RNA, 21 nt, Homo sapiens
agaggauacccuuuguauguu

SEQ ID NO: 881 - RNA, 22 nt, Homo sapiens
cggaugagcaaagaaagugguu

SEQ ID NO: 882 - RNA, 17 nt, Homo sapiens
uaagugcuuccaugcuu

SEQ ID NO: 883 - RNA, 20 nt, Homo sapiens
ucguuugccuuuuucugcuu

SEQ ID NO: 884 - RNA, 23 nt, Homo sapiens
aggaugagcaaagaaaguagauu

SEQ ID NO: 885 - RNA, 23 nt, Homo sapiens
cuggagauauggaagagcugugu

SEQ ID NO: 886 - RNA, 21 nt, Homo sapiens
uaggacacauggucuacuucu

SEQ ID NO: 887 - RNA, 18 nt, Homo sapiens
cagggaggugaaugugau

SEQ ID NO: 888 - RNA, 18 nt, Homo sapiens
ugaggcaguagauugaau

SEQ ID NO: 889 - RNA, 24 nt, Homo sapiens
ccagacagaauucuaugcacuuuc

SEQ ID NO: 890 - RNA, 22 nt, Homo sapiens
aaaaguaaucgcgguuuuuguc

SEQ ID NO: 891 - RNA, 24 nt, Homo sapiens
agccuggaagcuggagccugcagu

SEQ ID NO: 892 - RNA, 22 nt, Homo sapiens
aaaaguacuugcggauuuugcu

SEQ ID NO: 893 - RNA, 21 nt, Homo sapiens
acucuagcugccaaaggcgcu

SEQ ID NO: 894 - RNA, 22 nt, Homo sapiens
ucugggcaacaaagugagaccu

SEQ ID NO: 895 - RNA, 21 nt, Homo sapiens
aagugaucuaaaggccuacau

SEQ ID NO: 896 - RNA, 25 nt, Homo sapiens
ugggaacggguuccggcagacgcug

SEQ ID NO: 897 - RNA, 18 nt, Homo sapiens
ucagcuggcccucauuuc

SEQ ID NO: 898 - RNA, 24 nt, Homo sapiens
uugcagcugccugggagugacuuc

SEQ ID NO: 899 - RNA, 22 nt, Homo sapiens
ugcuggaucagugguucgaguc

SEQ ID NO: 900 - RNA, 22 nt, Homo sapiens
cuccugagccauucugagccuc

SEQ ID NO: 901 - RNA, 23 nt, Homo sapiens
gagggucuugggagggaugugac

SEQ ID NO: 902 - RNA, 21 nt, Homo sapiens
uggacugcccugaucuggaga

SEQ ID NO: 903 - RNA, 17 nt, Homo sapiens
ucccaccgcugccaccc

SEQ ID NO: 904 - RNA, 24 nt, Homo sapiens
uggcccugacugaagaccagcagu

SEQ ID NO: 905 - RNA, 17 nt, Homo sapiens
gugggggagaggcuguc

SEQ ID NO: 906 - RNA, 27 nt, Homo sapiens
cacuguaggugauggugagagugggca

SEQ ID NO: 907 - RNA, 23 nt, Homo sapiens
ccugcagcgacuugauggcuucc

SEQ ID NO: 908 - RNA, 20 nt, Homo sapiens
uaaagagcccuguggagaca

SEQ ID NO: 909 - RNA, 22 nt, Homo sapiens
aaaagcuggguugagagggcaa

SEQ ID NO: 910 - RNA, 26 nt, Homo sapiens
gaugaugauggcagcaaauucugaaa

SEQ ID NO: 911 - RNA, 22 nt, Homo sapiens
uuuccggcucgcgugggugugu

SEQ ID NO: 912 - RNA, 22 nt, Homo sapiens
aggcauugacuucucacuagcu

SEQ ID NO: 913 - RNA, 22 nt, Homo sapiens
uaguacugugcauaucaucuau

SEQ ID NO: 914 - RNA, 22 nt, Homo sapiens
augggugaauuuguagaaggau

SEQ ID NO: 915 - RNA, 22 nt, Homo sapiens
aacuggaucaauuauaggagug

SEQ ID NO: 916 - RNA, 22 nt, Homo sapiens
gguggcccggccgugccugagg

SEQ ID NO: 917 - RNA, 21 nt, Homo sapiens
gugccagcugcagugggggag

SEQ ID NO: 918 - RNA, 22 nt, Homo sapiens
agaaggaaauugaauucauuua

SEQ ID NO: 919 - RNA, 22 nt, Homo sapiens
uucauucggcuguccagaugua

SEQ ID NO: 920 - RNA, 21 nt, Homo sapiens
uuaggccgcagaucuggguga

SEQ ID NO: 921 - RNA, 19 nt, Homo sapiens
uggauuuuuggaucaggga

SEQ ID NO: 922 - RNA, 22 nt, Homo sapiens
uuuucaacucuaaugggagaga

SEQ ID NO: 923 - RNA, 22 nt, Homo sapiens
acgcccuucccccccuucuuca

SEQ ID NO: 924 - RNA, 27 nt, Homo sapiens
accuucuuguauaagcacugugcuaaa

SEQ ID NO: 925 - RNA, 23 nt, Homo sapiens
uggaguccaggaaucugcauuuu

SEQ ID NO: 926 - RNA, 21 nt, Homo sapiens
ucguggccuggucuccauuau

SEQ ID NO: 927 - RNA, 27 nt, Homo sapiens
auugaucaucgacacuucgaacgcaau

SEQ ID NO: 928 - RNA, 22 nt, Homo sapiens
uuugaggcuacagugagaugug

SEQ ID NO: 929 - RNA, 18 nt, Homo sapiens
gcaugggugguucagugg

SEQ ID NO: 930 - RNA, 21 nt, Homo sapiens
cccggagccaggaugcagcuc

SEQ ID NO: 931 - RNA, 21 nt, Homo sapiens
uguucauguagauguuuaagc

SEQ ID NO: 932 - RNA, 22 nt, Homo sapiens
aaaacuguaauuacuuuuguac

SEQ ID NO: 933 - RNA, 20 nt, Homo sapiens
ucacuguucagacaggcgga

SEQ ID NO: 934 - RNA, 22 nt, Homo sapiens
aaaaacugagacuacuuuugca

SEQ ID NO: 935 - RNA, 18 nt, Homo sapiens
gucccuguucaggcgcca

SEQ ID NO: 936 - RNA, 17 nt, Homo sapiens
ucccuguucgggcgcca

SEQ ID NO: 937 - RNA, 21 nt, Homo sapiens
ccuguugaaguguaaucccca

SEQ ID NO: 938 - RNA, 21 nt, Homo sapiens
acggugcuggauguggccuuu

SEQ ID NO: 939 - RNA, 22 nt, Homo sapiens
caaaaguaauuguggauuuugu

SEQ ID NO: 940 - RNA, 22 nt, Homo sapiens
ucuacaaaggaaagcgcuuucu

SEQ ID NO: 941 - RNA, 22 nt, Homo sapiens
acccgucccguucguccccgga

SEQ ID NO: 942 - RNA, 21 nt, Homo sapiens
agagaagaagaucagccugca

SEQ ID NO: 943 - RNA, 17 nt, Homo sapiens
ucucgcuggggccucca

SEQ ID NO: 944 - RNA, 18 nt, Homo sapiens
aucccaccucugccacca

SEQ ID NO: 945 - RNA, 23 nt, Homo sapiens
uauucauuuauccccagccuaca

SEQ ID NO: 946 - RNA, 21 nt, Homo sapiens
uugggacauacuuaugcuaaa

SEQ ID NO: 947 - RNA, 18 nt, Homo sapiens
uugagaaggaggcugcug

(19) United States
(12) Patent Application Publication
(10) Pub. No.: US 2013/0065778 A1
(43) Pub. Date: Mar. 14, 2013

(54) MicroRNA Signatures Predicting Responsiveness To Anti-HER2 Therapy

(71) Applicant: Joanne B. Weidhaas, Westport, CT (US)

(72) Inventor: Joanne B. Weidhaas, Westport, CT (US)

(73) Assignee: YALE UNIVERSITY, NEW HAVEN, CT (US)

(21) Appl. No.: 13/518,854

(22) PCT Filed: Jan. 26, 2011
(86) PCT No.: PCT/US11/22606

§ 371 Date: Nov. 30, 2012

Related U.S. Application Data
(60) Provisional application No. 61/298,454, filed on Jan. 26, 2010.

(51) Int. Cl. C40B 30/04; C40B 40/06; C12Q 1/68

(52) U.S. Cl. 506/9; 435/6.11; 506/16

(57) ABSTRACT

The invention provides miRNA signatures and methods of making and using thereof. MiRNA signatures determine the responsiveness of HER2 expressing breast tumors to anti-HER2 treatment, such as the targeted drug therapy trastuzumab.

### RELATED APPLICATIONS

This application is related to provisional application U.S. Ser. No. 61/298,454, filed Jan. 26, 2010, the contents which are herein incorporated by reference in their entirety.

### FIELD OF THE INVENTION

This invention relates generally to the fields of cancer and molecular biology. The invention provides methods for predicting the responsiveness of tumors and patients to anti-Her2 therapy.

### BACKGROUND OF THE INVENTION

One of the most recent advances in cancer treatment is the development of trastuzumab (Herceptin®), a humanized monoclonal antibody that targets HER2-positive breast cancer cells to inhibit cell growth. Unfortunately, 65-90% of metastatic breast cancers overexpressing HER2 are initially resistant to trastuzumab treatment. Furthermore, the majority of those that do respond develop resistance and disease progression within one year of treatment initiation. Adjuvant therapy with trastuzumab or other anti-HER2-therapy to manage microscopic disease is likely faced with similar resistance levels. As these anti-HER2 therapies have some documented cardiotoxicity, biomarkers that predict sensitivity or resistance to trastuzumab and anti-HER2 therapies are therefore increasingly important. MicroRNAs (miRNAs) are global RNA regulators that are emerging as important regulators of cell stress response and survival pathways with significance in human cancer. Particular miRNAs are deleted, amplified or mis-expressed in breast cancer, although specific miRNA misregulation that impacts response to trastuzumab and/or anti-HER2 therapies has never before been evaluated.

### SUMMARY OF THE INVENTION

Using the methods of the invention, miRNA expression patterns were evaluated in cell lines known to be initially sensitive or initially resistant to trastuzumab. Furthermore, methods of the invention were used to evaluate human HER2 over-expressing breast cancer patient samples treated with neoadjuvant Herceptin with known responses to treatment. These methods revealed a miRNA signature, known as the HER2-sensitivity signature, within several cell lines, comprising several miRNAs that are differentially expressed between cells that are initially sensitive and initially resistant to trastuzumab. Moreover, these methods identified a miRNA signature in HER2 positive breast cancer patients that significantly separates trastuzumab responders from trastuzumab non-responders. Thus, the invention provides both composition and methods demonstrating that miRNA expression patterns act as biomarkers of trastuzumab sensitivity or resistance. These discoveries will lead to future modification of treatment planning for patients with HER2 positive breast cancer as well as identify potential future targets for therapy.

Specifically, the invention provides a miRNA signature that indicates a HER2-positive breast cancer cell that is responsive to a HER2-targeted therapy, the signature comprising the determination of the decreased abundance of one or more miRNAs selected from the group consisting of hsa-miR-148a (SEQ ID NO: 92), hsa-miR-151 (SEQ ID NO: 205), hsa-miR-193a (SEQ ID NO: 114), hsa-miR-15b (SEQ ID NO: 27), hsa-miR-98 (SEQ ID NO: 191), hsa-miR-9 (SEQ ID NO: 196), hsa-miR-187 (SEQ ID NO: 109) compared to a HER2-positive breast cancer cell that is non-responsive to a HER2-targeted therapy, and the determination of the increased abundance of one or more miRNAs selected from the group consisting of hsa-miR-126 (SEQ ID NO: 76), hsa-miR-451 (SEQ ID NO: 271), and hsa-miR-218 (SEQ ID NO: 138) compared to a HER2-positive breast cancer cell that is non-responsive to a HER2-targeted therapy.

The invention also provides an miRNA signature that indicates a HER2-positive breast cancer cell that is responsive to a HER2-targeted therapy, the signature comprising the determination of the decreased abundance of one or more miRNAs selected from the group consisting of hsa-miR-148a (SEQ ID NO: 92), hsa-miR-151 (SEQ ID NO: 205), hsa-miR-193a (SEQ ID NO: 114), hsa-miR-15b (SEQ ID NO: 27), hsa-miR-98 (SEQ ID NO: 191), hsa-miR-9 (SEQ ID NO: 196), hsa-miR-187 (SEQ ID NO: 109) compared to a HER2-positive breast cancer cell that is non-responsive to a HER2-targeted therapy, or the determination of the increased abundance of one or more miRNAs selected from the group consisting of hsa-miR-126 (SEQ ID NO: 76), hsa-miR-451 (SEQ ID NO: 271), and hsa-miR-218 (SEQ ID NO: 138) compared to a HER2-positive breast cancer cell that is non-responsive to a HER2-targeted therapy.

Alternatively, or in addition, the invention provides an miRNA signature including the decreased expression of one or more miRNAs selected from the group consisting of hsa-miR-148a (SEQ ID NO: 92), hsa-miR-151 (SEQ ID NO: 205), hsa-miR-193a (SEQ ID NO: 114), hsa-miR-15b (SEQ ID NO: 27), hsa-miR-98 (SEQ ID NO: 191), hsa-miR-9 (SEQ ID NO: 196), hsa-miR-187 (SEQ ID NO: 109), and the increased expression of one or more miRNAs selected from the group consisting of hsa-miR-126 (SEQ ID NO: 76), hsa-miR-451 (SEQ ID NO: 271), and hsa-miR-218 (SEQ ID NO: 138), wherein the miRNA is isolated from a HER2-positive breast cancer cell and the miRNA signature indicates responsiveness to a HER-2 targeted therapy. In one aspect, the HER2-targeted therapy is Trastuzumab. In another aspect, the HER2-positive breast cancer cell is positive for a second hormone receptor. Exemplary hormone receptors include, but are not limited to, the estrogen receptor and the progesterone receptor.

The invention also provides a method of determining a miRNA signature that distinguishes between a HER2-positive breast tumor that is responsive to HER2-targeted therapy and a HER2-positive breast tumor that is non-responsive to HER2-targeted therapy, including: (a) obtaining a sample of HER2-positive breast cancer that is non-responsive to HER2-targeted therapy; (b) isolating a miRNA selected from the group consisting of hsa-miR-148a, hsa-miR-151, hsa-miR-193a, hsa-miR-15b, hsa-miR-98, hsa-miR-9, hsa-miR-187, hsa-miR-126, hsa-miR-451, and hsa-miR-218 from said non-responsive tumor; (c) determining the expression level of the isolated miRNA in said non-responsive sample; and (d) comparing the expression level of the isolated miRNA in said non-responsive sample a known expression level of the isolated miRNA in a HER2-positive breast tumor that is responsive to HER2-targeted therapy; wherein the presence of a statistically-significant difference between the observed expression level of the isolated miRNA and the known expression level of said miRNA specifies a miRNA signature that distinguishes between a HER2-positive breast tumor that is responsive to HER2-targeted therapy and a HER2-positive breast tumor that is non-responsive to HER2-targeted therapy. In one embodiment of this method, the statistically-significant difference is a decrease in the expression level of hsa-miR-126, hsa-miR-451, or hsa-miR-218 in the non-responsive sample compared to the known level. Alternatively, or in addition, the statistically-significant difference is an increase in the expression level of hsa-miR-148a, hsa-miR-151, hsa-miR-193a, hsa-miR-15b, hsa-miR-98, hsa-miR-9, or hsa-miR-187 in the non-responsive sample compared to the known level. The known expression level of the isolated miRNA is calculated, retrieved from a database, or obtained experimentally. In a preferred embodiment of this method, the HER2-targeted therapy is trastuzumab. The non-responsive breast tumor resides either in the breast or at a second location in the body, e.g. if the breast cancer has spread or metastasized.

In certain embodiments of this method, the determining step further includes normalizing the isolated miRNA expression level from the non-responsive sample to a control RNA. Alternatively, or in addition, this method further includes: (a) normalizing the isolated miRNA expression level from a HER2-positive breast tumor that is responsive to a HER2-targeted therapy to a control RNA; and (b) comparing the expression levels of the isolated miRNA from the non-responsive and responsive samples, wherein the presence of a statistically-significant difference between the expression levels of the isolated miRNA in the non-responsive and the responsive samples specifies a miRNA signature that distinguishes between a HER2-positive breast tumor that is responsive to HER2-targeted therapy and a HER2-positive breast tumor that is non-responsive to HER2-targeted therapy.

The invention further provides a method of predicting the responsiveness of a breast tumor to HER-2-targeted therapy, including detecting the presence or absence of the miRNA signature described herein in a sample from a breast tumor, wherein the presence of the miRNA signature within the sample indicates that the breast tumor is responsive to HER-2-targeted therapy. The presence of the signature can be determined by measuring the levels in the tumor sample of at least one (and preferably at least two, at least three, at least four, at least five, at least six, at least seven, at least eight, at least nine, or ten or more) miRNAs that are indicative of the presence or absence of the signature. In a preferred embodiment of this method, the HER-2-targeted therapy is trastuzumab. The breast tumor resides in the breast or at a second location in the body. In certain embodiments of this method, the detecting step further includes normalizing the miRNA expression level of the isolated miRNA to a control RNA.

In one aspect of the methods described herein, the control RNA is a non-coding RNA selected from the group consisting of transfer RNA (tRNA), small nuclear RNA (snRNA) and small nucleolar RNA (snoRNA). Alternatively, the control RNA is a non-coding RNA of between 45 and 200 nucleotides. In other aspects, the control RNA is highly- and invariably-expressed between a responsive and non-responsive breast tumor. The invention further provides a method of predicting the responsiveness of a breast tumor to HER-2-targeted therapy, including the steps of: (a) obtaining a sample of a breast tumor; (b) isolating a miRNA from the sample; (c) determining the expression level of the isolated miRNA; and (d) comparing the expression level of the isolated miRNA to expression level of said miRNA in the miRNA signature of claim 1, wherein replication of the miRNA signature within the sample indicates that the breast tumor is responsive to HER-2-targeted therapy. In a preferred embodiment, the HER-2-targeted therapy is trastuzumab. The breast tumor resides either in the breast or at a second location in the body, e.g. the breast cancer has spread or metastasized.

In certain embodiments of this method, the determining step further includes normalizing the miRNA expression level of the isolated miRNA to a control RNA. The control RNA is optionally RNU6B (SEQ ID NO: 213).

### BRIEF DESCRIPTION OF THE DRAWINGS

FIG. 1 is a schematic representation of the biogenesis of miRNAs.

FIG. 2A is a graph depicting the broad range of responses from highly-expressing HER-2 breast cancer cell lines to increasing concentrations of Herceptin.

FIG. 2B is a graph depicting the results of a growth assay separated by breast cancer cell lines and type of Herceptin treatment over time.

FIG. 3 is a graphical representation of a miRNA expression signature that discriminates between Herceptin sensitive and resistance cell lines. P-values represent the level of statistical significance for differences between sensitive and resistant cell lines.

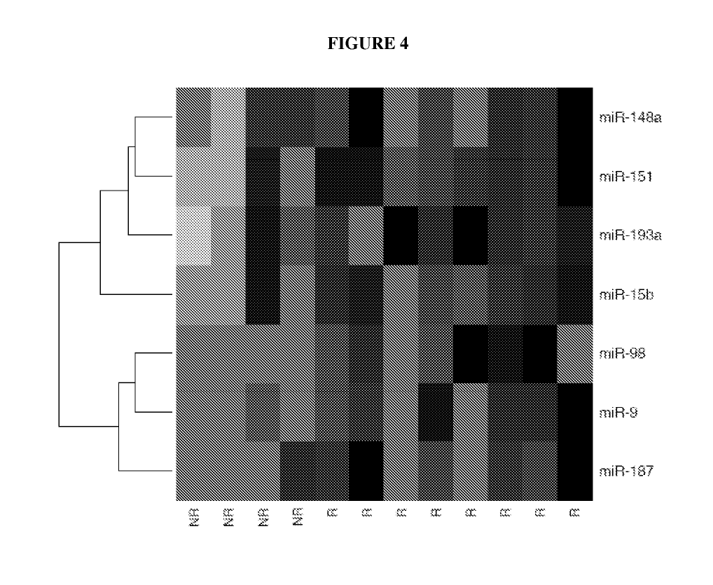

FIG. 4 is a graphical representation of a miRNA expression signature that discriminates between Herceptin sensitive and resistance cell lines.

FIG. 5A-G is a series of graphs depicting the mean and standard deviation for expression levels of individual miRNAs in cell lines that are resistant and not-resistant to Herceptin treatment.

FIG. 6 is a graphical representation of a miRNA expression signature that discriminates between Herceptin sensitive and resistance cell lines.

FIG. 7A-D is a series of graphs depicting the mean and standard deviation for expression levels of individual miRNAs in cell lines that are resistant and not-resistant to Herceptin treatment.

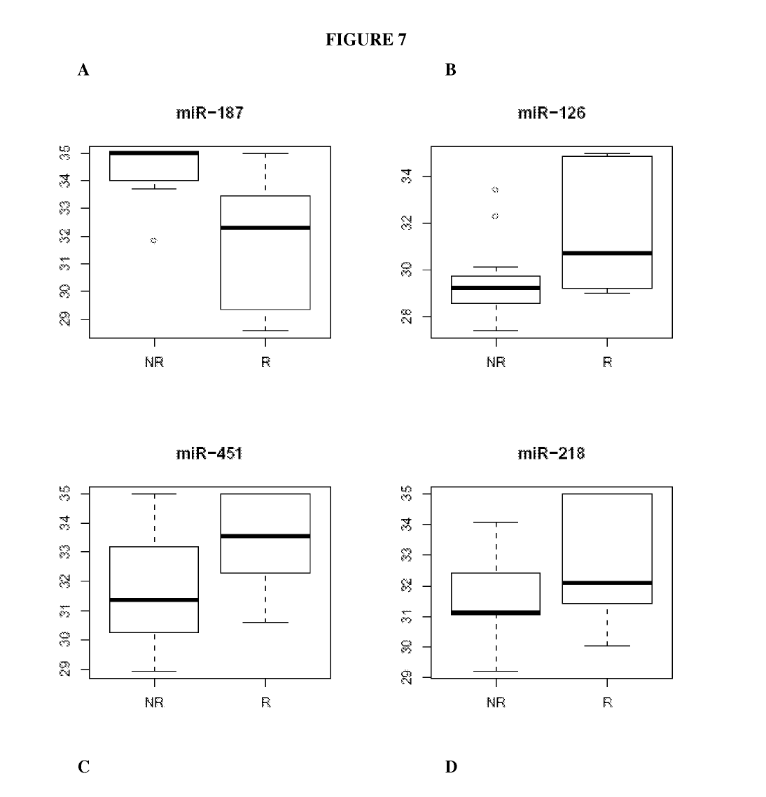

### DETAILED DESCRIPTION

HER2 is a receptor-like tyrosine kinase that is part of the family of epidermal group factor receptors (EGFR). The HER2 protein product (also called neu or ErbB2 for rat and mouse homologues, respectively) is present at high levels on the surface of cells in 25 to 30% of invasive breast carcinomas. This subgroup is referred to as HER2-overexpressing, or HER2-positive breast cancer, and has been historically associated with poor prognosis.

Approved for clinical use in 1998, Trastuzumab (also known as Herceptin®), is a humanized monoclonal antibody that binds specifically to the extracellular domain of HER2, inhibiting cell growth in HER2-positive cells. While the exact mechanism of Herceptin action is not completely understood, there are several proposed pathways. Herceptin treatment has been shown to decrease phosphorylated Akt levels and Akt kinase activity, as seen by the reduced phosphorylation of glycogen synthase kinase 3-β, a substrate of Akt (Yakes F M et al. (2002) Cancer Res 62: 4132-4141). This is significant because the phosphorylation of p27, an inhibitor of cdk2 and of cell proliferation, by AKT interferes with its translocation into the nucleus Inhibition of phosphorylation allows p27 to enter the nucleus and inhibit cdk2, therefore arresting the cell in the G1/G0 phase of the cell cycle (Yakes F M et al. (2002) Cancer Res 62: 4132-4141). Herceptin may also signal the immune system to destroy tumor cells expressing the HER2 protein, which is known as an antibody-dependent, cell-mediated cytotoxicity response (Gennari R et al. (2004) Clin Cancer Res 10: 5650-5655).

A crucial problem with this treatment is the prevalence of initial and developed resistance of the tumor to the drug. Approximately 65-90% of metastatic breast cancers overexpressing HER2 are initially resistant to Herceptin treatment, suggesting that HER2 amplification is necessary but not sufficient for Herceptin responsiveness (Cobleigh M A et al. (1999) Journal of Clinical Oncology 17: 719-726). Furthermore, the majority of tumors that do respond develop resistance and disease progression within one year of treatment initiation (Nahta R et al. (2006) Nature Clinical Practice Oncology 3 (5): 269-279). Therefore, research to identify those that will initially respond to or be resistant to Herceptin therapy is critical. Currently proposed mechanisms of resistance include down-regulation of p27, loss of PTEN activity, and activation of insulin-like growth factor I receptor (IGF-1R) (Camriand, A., Lu, Y. Pollak, M. Med. Sci. Mont. 2002 December; 8(12): BR521-6). Due to the high cost of treatment and potential side effects associated with Herceptin treatment, such as cardiac dysfunction, biomarkers that can predict response or more importantly resistance are necessary. Additionally, biomarkers giving insight into potential future therapies that could improve the effectiveness of Herceptin hold great clinical value.

MicroRNAs are a set of small endogenous non-protein-coding, regulatory RNAs that control the expression of multiple gene types, including genes involved in cell growth, differentiation and apoptosis (Iorio, M. V., et al. Cancer Research, 2005. 65: p. 7065-7070). miRNAs have been shown to be misregulated in all cancer types thus far studied, including breast cancer (Iorio, M. V., et al. Cancer Research, 2005. 65: p. 7065-7070). As molecular subtype classification has been well documented by gene expression profiling, it was clear that miRNAs should also segregate these groups. Recently a group was able to confirm that miRNA expression profiles do differentiate these three groups, confirming the genetic uniqueness of these forms of breast cancer. However, work by our group as well as others also indicates that miRNAs vary within cancer subtypes. Therefore, miRNA signatures of the invention predict outcome. miRNAs have been shown to be biomarkers of cancer outcome in numerous cancer types.

miRNAs have been found to be important in the cellular stress response, including the cellular response to cytotoxic therapy such as radiation and chemotherapy. miRNAs are dynamically altered in the stress response, suggesting that for those miRNAs critical in cell survival, different tumor levels may give selective survival advantages or disadvantages. This may in fact explain how miRNAs predict outcome in cancer, and their role in predicting response to treatment is continually being expanded.

It was hypothesized that miRNAs would be involved in the response to Herceptin, and thus, that initial miRNA levels in HER2 positive tumors would predict response to Herceptin treatment. Therefore, miRNA expression profiles were evaluated in HER2 positive cell lines known to be initially sensitive or resistant to Herceptin therapy. A miRNA profile was determined that clearly separated these cell lines into their respective response groups. Next, human HER2 positive tumor specimens were profiled from patients before Herceptin exposure, but with known responses. A miRNA signature was determined that significantly separated the Herceptin responders from the Herceptin no responders. These studies demonstrate that miRNA signatures can be used as biomarkers to predict response to Herceptin therapy in response to HER2 positive breast cancer. Furthermore, these studies suggest that miRNA signatures can be used as biomarkers to predict response to any HER2 targeted therapy in response to HER2 positive breast cancer.

### Cancer

Cancer is a group of many related diseases. All cancers begin in cells that make up the organs of the body. Normally, cells division is a regulated process throughout development and adulthood. Cells are instructed to grow and divide to form new cells only as the body needs them. For instance, when existing cells die, new cells are generated to replace them.

When cell division or cell proliferation becomes unregulated or misregulated, new cells form even when the body does not need them. Alternatively, or in addition, the lives of existing cells are prolonged because they do not engage in programmed cell death at the expected times. Tumors result from the resultant accumulation of cells that forms when cell proliferation and/or death becomes misregulated.

The term “tumor” is meant to describe an abnormal growth of body tissue resulting from a cell proliferative disorder, which is benign (non-cancerous), pre-malignant (pre-cancerous) or malignant (cancerous). Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell,” is defined as any cell that divides at a rate that exceeds, or is greater than, what is expected or observed among neighboring or juxtaposed cells within the same tissue.

Cancer cells can invade and damage nearby tissues and organs when they detach from the primary malignant tumor, enter the bloodstream or lymphatic system, and form new tumors in other organs. The spread of cancer is called metastasis. Cancers that are distinguished using the miRNA signatures and methods of the invention include, but are not limited to, breast cancer and all of its subtypes, such as ductal carcinoma, lobular carcinoma, in situ breast cancer (noninvasive), ductal carcinoma in situ, invasive (infiltrating) breast cancer, invasive ductal carcinoma (tubular, mucinous, medullary, and papillary), invasive lobular carcinoma, hormone receptor positive breast cancer, hormone receptor negative breast cancer, estrogen receptor (ER) positive breast cancer, estrogen receptor (ER) negative breast cancer, progesterone receptor (PR) positive breast cancer, progesterone receptor (PR) negative breast cancer, HER-2 positive breast cancer, HER-2 negative breast cancer, ER/PR/HER2 positive (triple positive) breast cancer, ER/PR/HER2 negative (triple negative) breast cancer, luminal A breast cancer, luminal B breast cancer, basal breast cancer.

A subject of the invention is preferably a mammal. The mammal can be a human, non-human primate, mouse, rat, dog, cat, horse, or cow, but are not limited to these examples. Mammals other than humans can be advantageously used as subjects that represent animal models of a particular disease. A subject can be male or female. A subject can be one who has been previously diagnosed or identified as having a disease and optionally has already undergone, or is undergoing, a therapeutic intervention for the disease. Alternatively, a subject can also be one who has not been previously diagnosed as having the disease. For example, a subject can be one who exhibits one or more risk factors for a disease. A subject is also a patient.

The biological or tumor sample can be any tissue or fluid that contains a nucleic acid. Various embodiments include paraffin imbedded tissue, frozen tissue, surgical fine needle aspirations, cells of the uterus, ovary, skin, muscle, lung, head and neck, esophagus, kidney, pancreas, mouth, throat, pharynx, larynx, esophagus, facia, brain, prostate, breast, endometrium, small intestine, blood cells, liver, testes, ovaries, uterus, cervix, colon, stomach, spleen, lymph node, or bone marrow. Other embodiments include fluid samples such as bronchial brushes, bronchial washes, bronchial ravages, peripheral blood lymphocytes, lymph fluid, ascites fluid, pleural effusion, sputum, cerebrospinal fluid, lacrimal fluid, esophageal washes, and stool or urinary specimens such as bladder washing and urine.

In certain embodiments, the miRNA signature and methods of the invention determines the responsiveness of a breast cancer cell, tumor, or subject to Herceptin therapy. For instance, a tumor biopsy is tested for cellular sensitivity to Herceptin prior to treatment of the subject or patient with Herceptin. Tumors or cells that are sensitive or responsive to Herceptin treatment fail to divide following treatment. Alternatively, or in addition, tumors or cells that are sensitive or responsive to Herceptin treatment undergo programmed cell death (also known as apoptosis) or necrosis following treatment.

The term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes).

The cancer stage which is present at diagnosis is the single-most important indicator of patient prognosis and survival. As such, patient treatment regimens are typically designed in response to the determination of cancer stage made at the time of diagnosis. Cancer staging is generally performed according to the Tumor, Node, Metastasis (TNM) System, which is the universally-accepted system of the Union Internationale Contre le Cancer (UICC) and the American Joint Committee on Cancer (AJCC). FIGO (Federation Internationale de Gynécologie et Obstétrique, International Federation of Gynecology and Obstetrics) is an international organization that defines staging systems in gynecological malignancy.

The TNM categories correspond with the FIGO staging system. The TNM system further denotes the stage of the cancer as either “clinical stage,” or “pathological stage.” The clinical stage, denoted by a “c” preceding the grade, is based upon all of the information obtainable prior to surgery including physical examination of the patient, radiologic examination, and endoscopy. Moreover, the pathological stage, denoted by a lower case “p” preceding the grade, is based upon all of the information gathered prior to surgery as well as additional information gained by pathological microscopic examination of the tumor. Although biopsy is used to remove tissue and perform clinical and pathological studies, surgical removal of the tumor is preferred. Biopsy can be performed according to a variety of methods, including, but not limited to, fine needle aspiration, core biopsy, and excision biopsy. Furthermore, this system includes a C-factor, or certainty factor, that reflects the validity of classification with respect to the diagnostic methods employed.

Overall Stage Grouping is also referred to as Roman Numeral Staging. This system uses numerals I, II, III, and IV (plus the 0) to describe the progression of cancer. Stage 0 is in situ carcinoma, a pre-invasive malignancy that does not invade the basement membrane and by definition does not metastasize. Stages I-III indicate increasingly severe conditions with increasing poor prognoses. Higher numbers indicate more extensive disease: greater tumor size, and/or spread of the cancer to nearby lymph nodes, and/or organs adjacent to the primary tumor. Typically, stage IV is metastatic cancer indicating that the cancer has spread to another distant organ.

Within the TNM system, a cancer may also be designated as recurrent, meaning that it has appeared again after being in remission or after all visible tumor has been eliminated. Recurrence can either be local, meaning that it appears in the same location as the original, or distant, meaning that it appears in a different part of the body.

The TNM system has more specific grades including the following primary tumor (T) grades: TX=Primary tumor cannot be evaluated, T0=No evidence of primary tumor, Tis=In situ carcinoma in situ, and T1-T4=increasing size and/or extent of the primary tumor. The TNM system further includes the following specific regional lymph node grades: NX=Regional lymph nodes (N) cannot be evaluated, N0=No regional lymph node involvement (no cancer found in the lymph nodes), and N1-N3=Increasing involvement of regional lymph nodes (number and/or extent of spread). Furthermore, the TNM system includes the following distant metastasis (M) grades: MX=Distant metastasis cannot be evaluated, MO=No distant metastasis (cancer has not spread to other parts of the body), and M1=Distant metastasis (cancer has spread to distant parts of the body).

Tumors are also graded according to histopathology and provided a histopathologic grade. Accordingly, the histopathologic grade is a qualitative assessment of the differentiation of the tumor expressed as the extent to which a tumor resembles normal tissue present at the site. Grade is expressed numerically from most differentiated (Grade 1) to least differentiated (Grade 4). Exemplary histopathologic grades include, but are not limited to, GX=histopathological grade cannot be determined, G1=well-differentiated, G2=moderately differentiated, G3=poorly differentiated, and G4=undifferentiated.

Histopathologic type is a qualitative pathologic assessment wherein the tumor is characterized or typed according to the normal tissue type of cell type it most closely resembles. In general, the World Health Organization International Histologic Classification of Tumors is for histopathologic typing (WHO International Classification of Diseases for Oncology ICD-O (3rd edition), World Health Organization, Geneva, 2000).

Alternatively, or in addition, severity is meant to describe the tumor grade by art-recognized methods (see, National Cancer Institute, www.cancer.gov). Tumor grade is a system used to classify cancer cells in terms of how abnormal the cells look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov). In another aspect of the invention, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized.

### Breast Cancer

Breast cancer forms in the cells of the breast. Although breast cancer is far more common in women, breast cancer can occur in both men and women.

A subject or patient may experience at least one signs or symptoms of breast cancer prior to or concurrent with diagnosis. Signs or symptoms of breast cancer include, but are not limited to, a breast lump or thickening that feels different from the surrounding tissue, bloody discharge from the nipple, a change in the size or shape of a breast, a change to the skin over the breast, such as dimpling, an inverted nipple, peeling or flaking of the nipple skin, and redness or pitting of the skin over the breast. A sign of breast cancer is typically observed by a medical professional, whereas a symptom of breast cancer is typically experienced by a patient or subject and reported to a medical professional.

Breast cancer is initiated when a portion of breast cells being growing abnormally. For instance, these cells divide more rapidly than healthy cells do or they divide at developmentally inappropriate times. The accumulating cells form a tumor that may spread, or metastasize, through the breast, to a lymph node or to other parts of the body. Breast cancer which has not spread or metastasized is a primary breast tumor. Primary breast cancer most frequently occurs in cells in the milk-producing ducts. This subtype of breast cancer is invasive ductal carcinoma. Alternatively, or in addition, primary breast cancer frequently occurs in the lobules (a subtype called invasive lobular carcinoma) or in the cells of the breast.

The severity of breast cancer is expressed by the tumor stage and grade. Tumor stage is determined according to the TNM system described herein (see Tables 1A and 1B)

TABLE 1A

| TNM | Stage | Definition |
|---|---|---|
| Primary Tumor | X | Primary tumor cannot be assessed T0 |
| (T) | | No evidence of primary tumor Tis: Carcinoma in situ; intraductal |
| | | carcinoma, or lobular carcinoma in situ |
| T | 1 | Tumor 2.0 cm or less in greatest dimension |
| | | T1mic: Microinvasion 0.1 cm or less in greatest dimension |
| | | T1a: Tumor more than 0.1 but not more than 0.5 cm in greatest |
| | | dimension |
| | | T1b: Tumor more than 0.5 cm but not more than 1.0 cm in greatest |
| | | dimension |
| | | T1c: Tumor more than 1.0 cm but not more than 2.0 cm in greatest |
| | | dimension |
| T | 2 | Tumor more than 2.0 cm but not more than 5.0 cm in greatest dimension |
| T | 3 | Tumor more than 5.0 cm in greatest dimension |
| T | 4 | Tumor of any size with direct extension to (a) chest wall or (b) skin, |
| | | only as described below. Chest wall includes ribs, intercostal muscles, |
| | | and serratus anterior muscle but not pectoral muscle. |
| | | T4a: Extension to chest wall |
| | | T4b: Edema (including peau d'orange) or ulceration of the skin of the |
| | | breast or satellite skin nodules confined to the same breast |
| | | T4c: Both of the above (T4a and T4b) |
| | | T4d: Inflammatory carcinoma* |
| Regional lymph | X | Regional lymph nodes cannot be assessed (e.g., previously removed) |
| nodes (N): | | |
| N | 0 | No regional lymph node metastasis |
| N | 1 | Metastasis to movable ipsilateral axillary lymph node(s) |
| N | 2 | Metastasis to ipsilateral axillary lymph node(s) fixed to each other or to |
| | | other structures |
| N | 3 | Metastasis to ipsilateral internal mammary lymph node(s) |
| Distant | X | Presence of distant metastasis cannot be assessed |
| metastasis (M): | | |
| M | 0 | No distant metastasis |
| M | 1 | Distant metastasis present (includes metastasis to ipsilateral |
| | | supraclavicular lymph nodes) |

TABLE 1B

| AJCC Stage | |
|---|---|
| Groupings | Description (TNM combinations from Table 1A) |
| Stage 0 | Tis, N0, M0 |
| Stage 1 | T1,* N0, M0 |
| | *T1 includes T1mic |
| Stage IIA | T0, N1, M0 T1,* N1, M0 T2, N0, M0 |
| | *T1 includes T1mic |
| Stage IIB | T2, N1, M0 |
| | T3, N0, M0 |
| Stage IIIA | T0, N2, M0 T1,* N2, M0 T2, N2, M0 T3, |
| | N1, M0 T3, N2, M0 |
| | *T1 includes T1mic |
| Stage IIIB | T4, Any N, M0 |
| | Any T, N3, M0 |
| Stage IV | Any T, Any N, M1 |

Subjects or patients who have an increased risk of developing breast cancer have one or more of the following characteristics: female gender, advanced age, a personal and/or family history of breast cancer, at least one genetic mutation (for instance, the BRCA1 or BRCA2 mutation in the BRCA gene or the LCS6 mutation in the KRAS gene), increased radiation exposure, obesity, early onset of mensis, later onset of menopause, giving birth to first-born after age 35, postmenopausal hormone therapy, and drinking alcohol.

Typical treatments for breast cancer include surgery, radiation therapy, chemotherapy, hormone-blocking therapy, and targeted drug therapy. At best, surgery involves removal of the breast cancer itself and a small margin of the surrounding tissue (lumpectomy). However, the lumpectomy procedure is typically available to those patients having smaller tumors that are easily separated from the surrounding tissue. If a patient has more advanced cancer, surgery commonly requires either removing the entire breast (mastectomy), which includes the lobules, ducts, fatty tissue and skin, or also removing the underlying muscle of the chest wall along with surrounding lymph nodes in the armpit (radial mastectomy). As described previously, surgical treatments may also involve removing one or more lymph nodes. For example, a sentinel node is removed for biopsy. The sentinel lymph node is that lymph node near the cancerous breast which receives drainage from the cancer. This lymph node is removed and tested for the presence of breast cancer cells to determine if the cancer is metatstatic. If no cancer is found within this lymph node, the chance of finding cancer in any of the remaining nodes is small and no other nodes need to be removed. However, if cancer is detected in the sentinel lymph node, then removal of axillary lymph nodes is performed, for instance the lymph nodes residing in the ipsilateral armpit to the cancerous breast. Determining if breast cancer has spread and to what extent the cancer may have spread is critical to determining a prognosis and treatment regime.

Side effects and risks of surgical treatment include, but are not limited to, bleeding and infection. Removal of lymph nodes increases the risk of swelling of the arm, also known as lymphedema, because the lymph fluid is not longer being drained by the excised lymph nodes.

Radiation therapy is either performed using external beam radiation, by which radiation penetrates the body or tumor from the outside, or, brachytherapy, by which radiation is implanted at the tumor site and the radiation penetrates the tumor from inside the body or inside the tumor. Radiation is often used in combination with surgery. Common side effects include, but are not limited to, fatigue, skin irritation, changes in breast tissue (swelling and hardening), lymphedema, osteoporosis, and damage to lungs and nerves.

Chemotherapy involves drugs that target rapidly dividing cells. Chemotherapy can be used to shrink the size of a tumor to make the tumor operable (neoadjuvant chemotherapy). Alternatively, or in addition, chemotherapy is often used after surgery (adjuvant systemic chemotherapy) to ensure that cancer does not return or spread. Moreover, chemotherapy is used to control cancer and minimize signs or symptoms of the cancer in the subject or patient. Unfortunately, chemotherapy has many well-known side effects that are specific to the individual drug or combination of drugs used. Typically, side effects include, but not limited to, hair loss, nausea, vomiting, fever and frequent infections.

Some types of breast cancers are sensitive to hormones such as estrogen and progesterone. Tumor biopsies can be tested for expression of estrogen and progesterone receptors to determine, in part, the sensitivity of a cancer to hormone-blocking therapy. Typically, if a cancer expresses high levels of hormone receptors and/or grows in response to estrogen or progesterone, hormone-blocking therapy is an effective treatment.

One class of hormone-blocking medications prevent hormones from attaching to cancer cells. Tamoxifen is a selective estrogen receptor modulator (SERM). SERMs act by blocking any estrogen present in the body from attaching to the estrogen receptor on the cancer cells, slowing the growth of tumors and killing tumor cells. Tamoxifen can be used in both pre- and postmenopausal women.

Another class of hormone-blocking medications arrest estrogen production after menopause. For instance, aromatase inhibitors block the action of an enzyme that converts androgens into estrogen. Specifically, aromatase inhibitors are effective only in postmenopausal women, and include commonly known drugs, such as, anastrozole (Arimidex), letrozole (Femara) and exemestane (Aromasin). A related treatment involves surgery (removal of the ovaries) or drugs that arrest hormone production in the ovaries also in postmenopausal women.

Side effects of hormone-blocking therapy vary, but commonly include the following symptoms, including but not limited to, hot flashes, vaginal dryness, decreased sex drive and mood changes. Aromatase inhibitors cause specific side effects including joint and muscle pain, as well as an increased risk of developing osteoporosis.

Provided the sometimes disabling side effects of the well-known and commonly-used breast cancer treatments, newer therapies have focused on targeted drugs. However, targeted drug therapies require some knowledge about the tumor cells and specific abnormalities that have caused those cells to transform from normal to cancerous cells.

The most commonly used targeted-drug treatments are Bevacizumab (Avastin), Lapatinib (Tykerb), and Trastuzumab (Herceptin). Bevacizumab is a humanized monoclonal antibody that recognizes and blocks vascular endothelial growth factor A (VEGF-A), which is secreted by cancer cells, and which attracts new blood vessels that provide vital oxygen and nourishment for the malignant tumor. Bevacizumab is approved for treatment of metastatic breast cancer. Bevacizumab inhibits the growth of blood vessels, which is part of the body's normal healing and maintenance. The process of generating new blood vessels (angiogenesis) is essential in wound healing, and as collateral circulation around blocked or atherosclerotic blood vessels. Bevacizumab may interfere with these normal processes or exacerbate existing conditions in patients, such as coronary artery disease (CAD) or peripheral artery disease (PAD). The main reported side effects are hypertension and a heightened risk of bleeding.

Lapatinib (Tykerb) is a small molecule that inhibits the tyrosine kinase activity of two oncogenes: EGFR (epidermal growth factor receptor) and HER2/neu (Human EGFR type 2). Lapatinib is used to specifically target HER2-positive advanced breast cancer. According to the FDA, Lapatinib is approved for use in advanced breast cancer, and, in fact, treatment with Lapatinib is reserved for women who have already tried trastuzumab and whose cancer has progressed.

Trastuzumab (Herceptin) is a monoclonal antibody that binds to and interferes with the function of the HER2/neu receptor. The HER proteins regulate the normal developmental processes of cell growth, survival, adhesion, migration, and differentiation. However, these functions are often amplified or weakened in cancer cells. In breast cancers, HER2 is constitutively active and causes breast cells to reproduce uncontrollably, causing breast cancer. Specifically, HER2 sends signals without a mitogen activating or binding to any receptor. HER2 signals promote invasion, survival and growth of blood vessels (angiogenesis) of cells.

Trastuzumab binds to the extracellular segment of the HER2/neu receptor. Cells treated with trastuzumab undergo arrest during the G1 phase of the cell cycle. Thus, trastuzumab treatment causes reduced cellular proliferation. Trastuzumab may also downregulate expression of HER2/neu in these cells. Trastuzumab further suppresses angiogenesis by inducing secretion of anti-angiogenic factors and repressing secretion of proangiogenic factors. Furthermore, antibodies, such as trastuzumab, when bound to a cell, induce immune cells to kill that cell. Consequently, trastuzumab induces antibody-dependent cell-mediated cytotoxicity (ADCC) in cancer cells.

Trastazumab has several side effects, including an effect on the heart. Trastuzumab is associated with cardiac dysfunction in 2-7% of cases. Approximately 10% of patients are unable to tolerate this drug because of pre-existing heart problems. Thus, physicians must balance the risk of recurrent cancer against the higher risk of death due to cardiac disease in this population.

### Responsiveness to Trastuzumab/Herceptin Treatment

It is well-established in the field of cancer biology, and particularly in breast cancer, that trastuzumab effectively inhibits and reverses deregulated HER2 signaling. However, if the breast cancer is not caused by HER2-overactivity, or is not dependent on this pathway, trastuzumab may not provide any beneficial effect. Thus, on balance, the side effects of treatment may actually cause harm to the patient. Predicting the responsiveness of a patient or a tumor to trastuzumab treatment is essential.

Currently, the medical community relies upon immunohistochemistry (IHC) and either silver, chromogenic or fluorescent in situ hybridization (SISH/CISH/FISH) to determined HER2 expression levels within tumor cells. Alternatively, HER-2 amplification can be detected by virtual karyotyping of a formalin-fixed paraffin embedded tumor. Virtual karyotyping has the added advantage of assessing copy number changes throughout the genome. Various PCR-based methodologies are also used.

Routine HER-2 status is determined by IHC. There are two FDA-approved commercial kits available, the Dako HercepTest and Ventana Pathway. These tests stratify expression levels into the following: 0 (<20,000 receptors per cell, no visible expression), 1+(˜100,000 receptors per cell, partial membrane staining, <10% of cells overexpressing HER-2), 2+(˜500,000 receptors per cell, light to moderate complete membrane staining, >10% of cells overexpressing HER-2), and 3+(˜2,000,000 receptors per cell, strong complete membrane staining, >10% of cells overexpressing HER-2). The presence of cytoplasmic expression is disregarded by these tests. Treatment with trastuzumab is recommended when the eHER-2 expression level is scored as 3+.

Fluorescent in situ hybridization (FISH) is the art-recognized “gold standard” technique for identifying patients who would benefit from trastuzumab. A combination of IHC and FISH is also accepted as a standard, whereby IHC scores of 0 and 1+ are negative (no trastuzumab treatment), scores of 3+ are positive (trastuzumab treatment), and score of 2+ (equivocal case) is confirmed by further FISH analysis to obtain a definitive treatment decision.

Critically, the recognized standard tests provide information regarding the expression level of the HER2 gene or corresponding protein, but these tests do not provide any information about the regulation of the protein or its downstream effectors. MiRNAs regulate gene and protein expression during development and cancer. The methods of the invention provide a miRNA signature that predicts the responsiveness of a tumor or tumor cell to trastuzumab treatment. This miRNA signature reflects gene and protein regulation within HER2 overexpressing cancer cells, and therefore, provides a level of accuracy that previous tests could not have envisioned.

### MicroRNA Signatures

miRNAs are a broad class of small non-protein-coding RNA molecules of approximately 22 nucleotides in length that function in posttranscriptional gene regulation by pairing to the mRNA of protein-coding genes. Recently, it has been shown that miRNAs play roles at human cancer loci with evidence that they regulate proteins known to be critical in survival pathways (Esquela-Kerscher, A. & Slack, F. J. Oncomirs—microRNAs with a role in cancer. Nat Rev Cancer 2006. 6, 259-69; Ambros, V. Cell 2001. 107, 823-6; Slack, F. J. and Weidhaas, J. B. Future Oncol 2006. 2, 73-82). Because miRNAs control many downstream targets, it is possible for them to act as novel targets for the treatment in cancer.

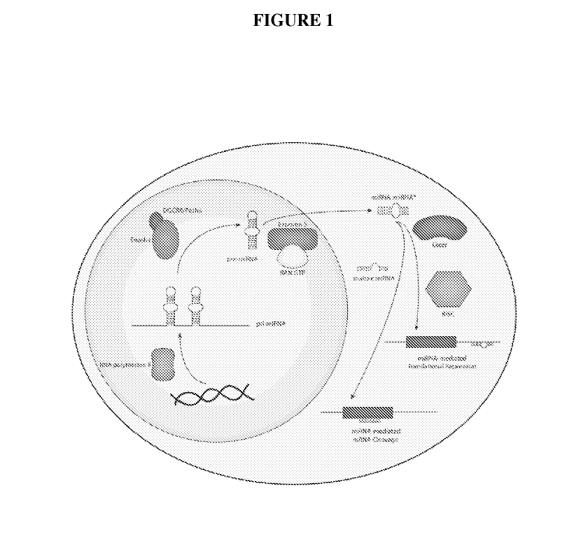

The basic synthesis and maturation of miRNAs can be visualized in FIG. 1 (Esquela-Kerscher, A. and Slack, F. J. Nat Rev Cancer 2006. 6, 259-69). In brief, miRNAs are transcribed from miRNA genes by RNA Polymerase II in the nucleus to form long primary RNAs (pri-miRNA) transcripts, which are capped and polyadenylated (Esquela-Kerscher, A. and Slack, F. J. Nat Rev Cancer 2006. 6, 259-69; Lee, Y. et al. Embo J 2002. 21, 4663-70). These pri-miRNAs can be several kilobases long, and are processed in the nucleus by the RNAaseIII enzyme Drosha and its cofactor, Pasha, to release the approximately 70-nucleotide stem-loop structured miRNA precursor (pre-miRNA). Pre-miRNAs are exported from the nucleus to the cytoplasm by exportin 5 in a Ran-guanosine triphosphate (GTP)-dependent manner, where they are then processed by Dicer, an RNase III enzyme. This causes the release of an approximately 22-base nucleotide, double-stranded, miRNA:miRNA duplex that is incorporated into a RNA-induced silencing complex (miRISC). At this point the complex is now capable of regulating its target genes.

FIG. 1 depicts how gene expression regulation can occur in one of two ways that depends on the degree of complementarity between the miRNA and its target. miRNAs that bind to mRNA targets with imperfect complementarity block target gene expression at the level of protein translation. Complementary sites for miRNAs using this mechanism are generally found in the 3′ UTR of the target mRNA genes. MiRNAs that bind to their mRNA targets with perfect complementarity induce target-mRNA cleavage. MiRNAs using this mechanism bind to miRNA complementary sites that are generally found in the coding sequence or open reading frame (ORF) of the mRNA target.

In mammals, miRNAs are gene regulators that are found at abnormal levels in virtually all cancer subtypes studied. Proper miRNA binding to their target genes is critical for regulating the mRNA level and protein expression.

The invention provides method of assessing the expression levels of, for instance, the miRNAs provided in Table 2. The ordinarily skilled artisan would readily recognize that the human miRNAs on this list are nonlimiting examples of miRNAs expressed in cancerous cells (miRNAs beginning with the letters “hsa”), as well as RNAs, which are useful as controls for real-time polymerase chain reaction (RT-PCR) (miRNAs not beginning with the letters “hsa”), as described above.

To generate a miRNA signature to distinguish between one or more cancer subtypes, the relative expression levels of one or more miRNAs present in the cancer cells of each subtype are determined with respect to a control RNA of known abundance. Alternatively, or in addition, the absolute expression levels of miRNAs are determined through a calculation that compares the relative levels to the known control level. Moreover, relative expression levels of miRNAs present in the cancer cells of each subtype are normalized to a highly- and invariably-expressed control RNA. The term “invariably-expressed RNA” is meant to describe an RNA, of which the expression level and pattern is similar in each of the tissues from which the compared cancer subtypes arise. Expression patterns are both spatial and temporal. The normalized miRNA expression levels can be further compared between one or more cancer subtypes. miRNAs that are expressed in one or more of the cancer subtypes would be included in a cancer subtype-specific miRNA signature; exclusive expression in one subtype over another is not required. However, when an miRNA of an miRNA signature is expressed in more than one cancer subtype, the expression level of that miRNA is preferably statistically significantly different, as determined by a p-value of 0.1 or less. Preferably, a p-value is 0.05 or less, or even more preferred are p-values of 0.01 or less.

TABLE 2
Experimental and Control Human miRNAs

| SEQ | | |
|---|---|---|
| ID | | miRBase ™ |
| NO: | Sequence | ID |
| 14 | UGAGGUAGUAGGUUGUAUAGUU | hsa-let-7a |
| 15 | UGAGGUAGUAGGUUGUGUGGUU | hsa-let-7b |
| 16 | UGAGGUAGUAGGUUGUAUGGUU | hsa-let-7c |
| 17 | AGAGGUAGUAGGUUGCAUAGU | hsa-let-7d |
| 18 | UGAGGUAGGAGGUUGUAUAGU | hsa-let-7e |
| 19 | UGAGGUAGUAGAUUGUAUAGUU | hsa-let-7f |
| 20 | UGAGGUAGUAGUUUGUACAGU | hsa-let-7g |
| 21 | UGAGGUAGUAGUUUGUGCUGU | hsa-let-7i |
| 22 | UGGAAUGUAAAGAAGUAUGUA | hsa-miR-1 |
| 23 | UGGAAGACUAGUGAUUUUGUUG | hsa-miR-7 |
| 24 | UACCCUGUAGAUCCGAAUUUGUG | hsa-miR-10a |
| 25 | UACCCUGUAGAACCGAAUUUGU | hsa-miR-10b |
| 26 | UAGCAGCACAUAAUGGUUUGUG | hsa-miR-15a |
| 27 | UAGCAGCACAUCAUGGUUUACA | hsa-miR-15b |
| 28 | UAGCAGCACGUAAAUAUUGGCG | hsa-miR-16 |
| 29 | ACUGCAGUGAAGGCACUUGU | hsa-miR-17-3p |
| 30 | CAAAGUGCUUACAGUGCAGGUAGU | hsa-miR-17-5p |
| 31 | UAAGGUGCAUCUAGUGCAGAUA | hsa-miR-18a |
| 3 | UGUGCAAAUCUAUGCAAAACUGA | hsa-miR-19a |
| 8 | UGUGCAAAUCCAUGCAAAACUGA | hsa-miR-19b |
| 32 | UAGCUUAUCAGACUGAUGUUGA | hsa-miR-21 |
| 33 | AAGCUGCCAGUUGAAGAACUGU | hsa-miR-22 |
| 34 | AUCACAUUGCCAGGGAUUUCC | hsa-miR-23a |
| 35 | AUCACAUUGCCAGGGAUUACC | hsa-miR-23b |
| 6 | UGGCUCAGUUCAGCAGGAACAG | hsa-miR-24 |
| 36 | CAUUGCACUUGUCUCGGUCUGA | hsa-miR-25 |
| 37 | UUCAAGUAAUCCAGGAUAGGC | hsa-miR-26a |
| 38 | UUCAAGUAAUCCAGGAUAGGCU | hsa-miR-26a |
| 39 | UUCAAGUAAUUCAGGAUAGGUU | hsa-miR-26b |
| 40 | UUCAAGUAAUUCAGGAUAGGU | hsa-miR-26b |
| 41 | UUCACAGUGGCUAAGUUCCGC | hsa-miR-27a |
| 42 | UUCACAGUGGCUAAGUUCUGC | hsa-miR-27b |
| 43 | AAGGAGCUCACAGUCUAUUGAG | hsa-miR-28 |
| 44 | UAGCACCAUCUGAAAUCGGUU | hsa-miR-29a |
| 45 | UAGCACCAUUUGAAAUCAGUGUU | hsa-miR-29b |
| 46 | UAGCACCAUUUGAAAUCGGU | hsa-miR-29c |
| 47 | CUUUCAGUCGGAUGUUUGCAGC | hsa-miR-30a-3p |
| 48 | UGUAAACAUCCUCGACUGGAAG | hsa-miR-30a-5p |
| 49 | UGUAAACAUCCUACACUCUCAGC | hsa-miR-30c |
| 50 | UGUAAACAUCCCCGACUGGAAG | hsa-miR-30d |
| 51 | UGUAAACAUCCUUGACUGGA | hsa-miR-30e-5p |
| 52 | CUUUCAGUCGGAUGUUUACAGC | hsa-miR-30e-3p |
| 53 | UAUUGCACAUUACUAAGUUGC | hsa-miR-32 |
| 54 | GUGCAUUGUAGUUGCAUUG | hsa-miR-33 |
| 55 | UGGCAGUGUCUUAGCUGGUUGUU | hsa-miR-34a |
| 56 | UGGCAGUGUCUUAGCUGGUUGU | hsa-miR-34a |
| 57 | UAGGCAGUGUCAUUAGCUGAUUG | hsa-miR-34b |
| 58 | AGGCAGUGUAGUUAGCUGAUUGC | hsa-miR-34c |
| 59 | UAUUGCACUUGUCCCGGCCUG | hsa-miR-92 |
| 60 | UAUUGCACUUGUCCCGGCCUGU | hsa-miR-92a |
| 61 | AAAGUGCUGUUCGUGCAGGUAG | hsa-miR-93 |
| 62 | UUCAACGGGUAUUUAUUGAGCA | hsa-miR-95 |
| 63 | UUUGGCACUAGCACAUUUUUGC | hsa-miR-96 |
| 64 | AACCCGUAGAUCCGAUCUUGUG | hsa-miR-99a |
| 65 | CACCCGUAGAACCGACCUUGCG | hsa-miR-99b |
| 66 | AACCCGUAGAUCCGAACUUGUG | hsa-miR-100 |
| 67 | UACAGUACUGUGAUAACUGAAG | hsa-miR-101 |
| 68 | AGCAGCAUUGUACAGGGCUAUGA | hsa-miR-103 |
| 69 | UCAAAUGCUCAGACUCCUGU | hsa-miR-105 |
| 70 | UAAAGUGCUGACAGUGCAGAU | hsa-miR-106b |
| 71 | AGCAGCAUUGUACAGGGCUAUCA | hsa-miR-107 |
| 72 | UGGAGUGUGACAAUGGUGUUUGU | hsa-miR-122a |
| 73 | UUAAGGCACGCGGUGAAUGCCA | hsa-miR-124a |
| 74 | UCCCUGAGACCCUUUAACCUGUG | hsa-miR-125a |
| 75 | UCCCUGAGACCCUAACUUGUGA | hsa-miR-125b |
| 76 | UCGUACCGUGAGUAAUAAUGC | hsa-miR-126 |
| 77 | CAUUAUUACUUUUGGUACGCG | hsa-miR-126* |
| 78 | UCGGAUCCGUCUGAGCUUGGCU | hsa-miR-127 |
| 79 | UCACAGUGAACCGGUCUCUUUU | hsa-miR-128a |
| 80 | CAGUGCAAUGUUAAAAGGGCAU | hsa-miR-130a |
| 81 | CAGUGCAAUGAUGAAAGGGCAU | hsa-miR-130b |
| 82 | UAACAGUCUACAGCCAUGGUCG | hsa-miR-132 |
| 83 | UUGGUCCCCUUCAACCAGCUGU | hsa-miR-133a |
| 84 | UGUGACUGGUUGACCAGAGGG | hsa-miR-134 |
| 85 | UAUGGCUUUUUAUUCCUAUGUGA | hsa-miR-135a |
| 86 | UAUGGCUUUUCAUUCCUAUGUG | hsa-miR-135b |
| 87 | AGUGGUUUUACCCUAUGGUAG | hsa-miR-140 |
| 1 | UAACACUGUCUGGUAAAGAUGG | hsa-miR-141 |
| 5 | UGUAGUGUUUCCUACUUUAUGGA | hsa-miR-142-3p |
| 7 | CAUAAAGUAGAAAGCACUAC | hsa-miR-142-5p |
| 88 | UGAGAUGAAGCACUGUAGCUCA | hsa-miR-143 |
| 89 | GUCCAGUUUUCCCAGGAAUCCCUU | hsa-miR-145 |
| 90 | UGAGAACUGAAUUCCAUGGGUU | hsa-miR-146a |
| 91 | GUGUGUGGAAAUGCUUCUGC | hsa-miR-147 |
| 92 | UCAGUGCACUACAGAACUUUGU | hsa-miR-148a |
| 93 | UCAGUGCAUCACAGAACUUUGU | hsa-miR-148b |
| 94 | UCUGGCUCCGUGUCUUCACUCC | hsa-miR-149 |
| 95 | UCUCCCAACCCUUGUACCAGUG | hsa-miR-150 |
| 96 | UCAGUGCAUGACAGAACUUGGG | hsa-miR-152 |
| 97 | UCAGUGCAUGACAGAACUUGG | hsa-miR-152 |
| 98 | UUGCAUAGUCACAAAAGUGA | hsa-miR-153 |
| 100 | UAGGUUAUCCGUGUUGCCUUCG | hsa-miR-154 |
| 101 | AAUCAUACACGGUUGACCUAUU | hsa-miR-154* |
| 4 | UUAAUGCUAAUCGUGAUAGGGG | hsa-miR-155 |
| 103 | AACAUUCAACGCUGUCGGUGAGU | hsa-miR-181a |
| 104 | AACAUUCAACCUGUCGGUGAGU | hsa-miR-181c |
| 105 | UGGUUCUAGACUUGCCAACUA | hsa-miR-182* |
| 106 | UAUGGCACUGGUAGAAUUCACUG | hsa-miR-183 |
| 107 | UGGACGGAGAACUGAUAAGGGU | hsa-miR-184 |
| 108 | CAAAGAAUUCUCCUUUUGGGCUU | hsa-miR-186 |
| 109 | UCGUGUCUUGUGUUGCAGCCG | hsa-miR-187 |
| 110 | GUGCCUACUGAGCUGAUAUCAGU | hsa-miR-189 |
| 111 | UGAUAUGUUUGAUAUAUUAGGU | hsa-miR-190 |
| 112 | CAACGGAAUCCCAAAAGCAGCU | hsa-miR-191 |
| 113 | CUGACCUAUGAAUUGACAGCC | hsa-miR-192 |
| 114 | AACUGGCCUACAAAGUCCCAG | hsa-miR-193a |
| 115 | UGUAACAGCAACUCCAUGUGGA | hsa-miR-194 |
| 116 | UAGCAGCACAGAAAUAUUGGC | hsa-miR-195 |
| 117 | UAGGUAGUUUCAUGUUGUUGG | hsa-miR-196a |
| 118 | UAGGUAGUUUCCUGUUGUUGG | hsa-miR-196b |
| 119 | UUCACCACCUUCUCCACCCAGC | hsa-miR-197 |
| 120 | CCCAGUGUUCAGACUACCUGUUC | hsa-miR-199a |
| 121 | UACAGUAGUCUGCACAUUGGUU | hsa-miR-199a* |
| 122 | CCCAGUGUUUAGACUAUCUGUUC | hsa-miR-199b |
| 123 | UAACACUGUCUGGUAACGAUGU | hsa-miR-200a |
| 124 | UAAUACUGCCGGGUAAUGAUGG | hsa-miR-200c |
| 125 | GUGAAAUGUUUAGGACCACUAG | hsa-miR-203 |
| 126 | UUCCCUUUGUCAUCCUAUGCCU | hsa-miR-204 |
| 127 | UCCUUCAUUCCACCGGAGUCUG | hsa-miR-205 |
| 128 | UGGAAUGUAAGGAAGUGUGUGG | hsa-miR-206 |
| 129 | AUAAGACGAGCAAAAAGCUUGU | hsa-miR-208 |
| 130 | CUGUGCGUGUGACAGCGGCUGA | hsa-miR-210 |
| 131 | UUCCCUUUGUCAUCCUUCGCCU | hsa-miR-211 |
| 132 | UAACAGUCUCCAGUCACGGCC | hsa-miR-212 |
| 133 | ACCAUCGACCGUUGAUUGUACC | hsa-miR-213 |
| 134 | ACAGCAGGCACAGACAGGCAG | hsa-miR-214 |
| 135 | AUGACCUAUGAAUUGACAGAC | hsa-miR-215 |
| 136 | UAAUCUCAGCUGGCAACUGUG | hsa-miR-216 |
| 137 | UACUGCAUCAGGAACUGAUUGGAU | hsa-miR-217 |
| 138 | UUGUGCUUGAUCUAACCAUGU | hsa-miR-218 |
| 139 | UGAUUGUCCAAACGCAAUUCU | hsa-miR-219 |
| 140 | CCACACCGUAUCUGACACUUU | hsa-miR-220 |
| 141 | AGCUACAUUGUCUGCUGGGUUUC | hsa-miR-221 |
| 142 | AGCUACAUCUGGCUACUGGGUCUC | hsa-miR-222 |
| 11 | UGUCAGUUUGUCAAAUACCCC | hsa-miR-223 |
| 143 | AGGGCCCCCCCUCAAUCCUGU | hsa-miR-296 |
| 144 | CAGUGCAAUAGUAUUGUCAAAGC | hsa-miR-301 |
| 145 | UAAGUGCUUCCAUGUUUUGGUGA | hsa-miR-302a |
| 146 | UAAACGUGGAUGUACUUGCUUU | hsa-miR-302a* |
| 147 | UAAGUGCUUCCAUGUUUUAGUAG | hsa-miR-302b |
| 148 | ACUUUAACAUGGAAGUGCUUUCU | hsa-miR-302b* |
| 149 | UAAGUGCUUCCAUGUUUCAGUGG | hsa-miR-302c |
| 150 | UUUAACAUGGGGGUACCUGCUG | hsa-miR-302c* |
| 151 | UAAGUGCUUCCAUGUUUGAGUGU | hsa-miR-302d |
| 152 | AAAAGCUGGGUUGAGAGGGCGAA | hsa-miR-320 |
| 153 | GCACAUUACACGGUCGACCUCU | hsa-miR-323 |
| 154 | CGCAUCCCCUAGGGCAUUGGUGU | hsa-miR-324-5p |
| 155 | CCUAGUAGGUGUCCAGUAAGUGU | hsa-miR-325 |
| 156 | CCUCUGGGCCCUUCCUCCAG | hsa-miR-326 |
| 157 | CUGGCCCUCUCUGCCCUUCCGU | hsa-miR-328 |
| 158 | GCAAAGCACACGGCCUGCAGAGA | hsa-miR-330 |
| 159 | GCCCCUGGGCCUAUCCUAGAA | hsa-miR-331 |
| 160 | UCAAGAGCAAUAACGAAAAAUGU | hsa-miR-335 |
| 161 | UCCAGCUCCUAUAUGAUGCCUUU | hsa-miR-337 |
| 162 | UCCAGCAUCAGUGAUUUUGUUGA | hsa-miR-338 |
| 163 | UCCCUGUCCUCCAGGAGCUCA | hsa-miR-339 |
| 164 | UCCGUCUCAGUUACUUUAUAGCC | hsa-miR-340 |
| 165 | UCUCACACAGAAAUCGCACCCGUC | hsa-miR-342 |
| 166 | UGCUGACUCCUAGUCCAGGGC | hsa-miR-345 |
| 167 | UGUCUGCCCGCAUGCCUGCCUCU | hsa-miR-346 |
| 168 | UUAUCAGAAUCUCCAGGGGUAC | hsa-miR-361 |
| 169 | AAUUGCACUUUAGCAAUGGUGA | hsa-miR-367 |
| 170 | ACAUAGAGGAAAUUCCACGUUU | hsa-miR-368 |
| 171 | AAUAAUACAUGGUUGAUCUUU | hsa-miR-369-3p |
| 172 | GCCUGCUGGGGUGGAACCUGG | hsa-miR-370 |
| 173 | GUGCCGCCAUCUUUUGAGUGU | hsa-miR-371 |
| 174 | AAAGUGCUGCGACAUUUGAGCGU | hsa-miR-372 |
| 175 | GAAGUGCUUCGAUUUUGGGGUGU | hsa-miR-373 |
| 176 | ACUCAAAAUGGGGGCGCUUUCC | hsa-miR-373* |
| 177 | UUAUAAUACAACCUGAUAAGUG | hsa-miR-374 |
| 178 | UUUGUUCGUUCGGCUCGCGUGA | hsa-miR-375 |
| 179 | AUCAUAGAGGAAAAUCCACGU | hsa-miR-376a |
| 180 | AUCACACAAAGGCAACUUUUGU | hsa-miR-377 |
| 181 | CUCCUGACUCCAGGUCCUGUGU | hsa-miR-378 |
| 182 | UGGUAGACUAUGGAACGUA | hsa-miR-379 |
| 183 | UAUGUAAUAUGGUCCACAUCUU | hsa-miR-380-3p |
| 184 | UGGUUGACCAUAGAACAUGCGC | hsa-miR-380-5p |
| 185 | UAUACAAGGGCAAGCUCUCUGU | hsa-miR-381 |
| 186 | GAAGUUGUUCGUGGUGGAUUCG | hsa-miR-382 |
| 187 | AGAUCAGAAGGUGAUUGUGGCU | hsa-miR-383 |
| 188 | AUUCCUAGAAAUUGUUCAUA | hsa-miR-384 |
| 189 | CUGGACUUGGAGUCAGAAGGCC | hsa-miR-422b |
| 190 | AGCUCGGUCUGAGGCCCCUCAG | hsa-miR-423 |
| 191 | UGAGGUAGUAAGUUGUAUUGUU | hsa-miR-98 |
| 192 | AAAAGUGCUUACAGUGCAGGUAGC | hsa-miR-106a |
| 193 | CCACUGCCCCAGGUGCUGCUGG | hsa-miR-324-3p |
| 194 | UAAAGUGCUUAUAGUGCAGGUAG | hsa-miR-20a |
| 195 | GGUCCAGAGGGGAGAUAGG | hsa-miR-198 |
| 196 | UCUUUGGUUAUCUAGCUGUAUGA | hsa-miR-9 |
| 197 | UAAAGCUAGAUAACCGAAAGU | hsa-miR-9* |
| 198 | UAGCACCAUUUGAAAUCGGUUA | hsa-miR-29c |
| 199 | UCACAGUGAACCGGUCUCUUUC | hsa-miR-128b |
| 200 | CUUUUUGCGGUCUGGGCUUGC | hsa-miR-129 |
| 201 | UUGGUCCCCUUCAACCAGCUA | hsa-miR-133b |
| 202 | ACUCCAUUUGUUUUGAUGAUGGA | hsa-miR-136 |
| 203 | UAUUGCUUAAGAAUACGCGUAG | hsa-miR-137 |
| 204 | AGCUGGUGUUGUGAAUC | hsa-miR-138 |
| 205 | ACUAGACUGAAGCUCCUUGAGG | hsa-miR-151 |
| 206 | UUUGGCAAUGGUAGAACUCACA | hsa-miR-182 |
| 207 | UGGAGAGAAAGGCAGUUC | hsa-miR-185 |
| 208 | CAAGUCACUAGUGGUUCCGUUUA | hsa-miR-224 |
| 209 | UGGUUUACCGUCCCACAUACAU | hsa-miR-299-5p |
| 210 | UGUAAACAUCCUACACUCAGCU | hsa-miR-30b |
| 211 | CUGGACUUAGGGUCAGAAGGCC | hsa-miR-422a |
| 212 | CAGCAGCAAUUCAUGUUUUGAA | hsa-miR-424 |
| 213 | CGCAAGGAUGACACGCAAAUUCGUGAAGCGUUCCAUA | RNU6B |
| | UUUUU | |
| 218 | CAUCCCUUGCAUGGUGGAGGGU | hsa-miR-188 |
| 219 | UAAGGUGCAUCUAGUGCAGUUA | hsa-miR-18b |
| 220 | AACUGGCCCUCAAAGUCCCGCUUU | hsa-miR-193b |
| 221 | CAUCUUACCGGACAGUGCUGGA | hsa-miR-200a* |
| 222 | AGAGGUAUAGGGCAUGGGAAAA | hsa-miR-202 |
| 223 | UUUCCUAUGCAUAUACUUCUUU | hsa-miR-202* |
| 224 | CAAAGUGCUCAUAGUGCAGGUAG | hsa-miR-20b |
| 225 | UAUGUGGGAUGGUAAACCGCUU | hsa-miR-299-3p |
| 226 | UAAUGCCCCUAAAAAUCCUUAU | hsa-miR-365 |
| 227 | AGAUCGACCGUGUUAUAUUCGC | hsa-miR-369-5p |
| 228 | AGGUUACCCGAGCAACUUUGCA | hsa-miR-409-5p |
| 229 | ACUUCACCUGGUCCACUAGCCGU | hsa-miR-412 |
| 230 | UAAUACUGUCUGGUAAAACCGU | hsa-miR-429 |
| 231 | UCUUGGAGUAGGUCAUUGGGUGG | hsa-miR-432 |
| 232 | CUGGAUGGCUCCUCCAUGUCU | hsa-miR-432* |
| 233 | AUCAUGAUGGGCUCCUCGGUGU | hsa-miR-433 |
| 234 | UUGCAUAUGUAGGAUGUCCCAU | hsa-miR-448 |
| 235 | UGGCAGUGUAUUGUUAGCUGGU | hsa-miR-449 |
| 236 | UUUUUGCGAUGUGUUCCUAAUA | hsa-miR-450 |
| 237 | UGUUUGCAGAGGAAACUGAGAC | hsa-miR-452 |
| 238 | UCAGUCUCAUCUGCAAAGAAG | hsa-miR-452* |
| 239 | GAGGUUGUCCGUGGUGAGUUCG | hsa-miR-453 |
| 240 | AGAGGCUGGCCGUGAUGAAUUC | hsa-miR-485-5p |
| 241 | CAACCUGGAGGACUCCAUGCUG | hsa-miR-490 |
| 242 | AGUGGGGAACCCUUCCAUGAGGA | hsa-miR-491 |
| 245 | AGGACCUGCGGGACAAGAUUCUU | hsa-miR-492 |
| 246 | UUGUACAUGGUAGGCUUUCAUU | hsa-miR-493 |
| 247 | UGAAACAUACACGGGAAACCUCUU | hsa-miR-494 |
| 248 | AUUACAUGGCCAAUCUC | hsa-miR-496 |
| 249 | CAGCAGCACACUGUGGUUUGU | hsa-miR-497 |
| 250 | UUUCAAGCCAGGGGGCGUUUUUC | hsa-miR-498 |
| 251 | UUAAGACUUGCAGUGAUGUUUAA | hsa-miR-499 |
| 252 | AUGCACCUGGGCAAGGAUUCUG | hsa-miR-500 |
| 253 | AAUCCUUUGUCCCUGGGUGAGA | hsa-miR-501 |
| 254 | UAGCAGCGGGAACAGUUCUGCAG | hsa-miR-503 |
| 255 | GUCAACACUUGCUGGUUUCCUC | hsa-miR-505 |
| 256 | UAAGGCACCCUUCUGAGUAGA | hsa-miR-506 |
| 257 | UUUUGCACCUUUUGGAGUGAA | hsa-miR-507 |
| 258 | UGAUUGUAGCCUUUUGGAGUAGA | hsa-miR-508 |
| 259 | UGAUUGGUACGUCUGUGGGUAGA | hsa-miR-509 |
| 260 | UGGUAUUGCCAUUGCUUCACUGUUGGCUUUGACCAGG | Z30 |
| | GUAUGAUCUCUUAAUCUUCUCUCUGAGCUG | |
| 261 | CGCAAGGAUGACACGCAAAUUCGUGAAGCGUUCCAUA | RNU6B |
| | UUUUU | |
| 262 | CCUGGAUGAUGAUAGCAAAUGCUGACUGAACAUGAAG | RNU44 |
| | GUCUUAAUUAGCUCUAACUGACU | |
| 263 | GAACUUAUUGACGGGCGGACAGAAACUGUGUGCUGAU | RNU43 |
| | UGUCACGUUCUGAUU | |
| 264 | UCUACAGUGCACGUGUCU | hsa-miR-139 |
| 2 | UGAGAACUGAAUUCCAUAGGCU | hsa-miR-146b-5p |
| 265 | AACAUUCAUUGCUGUCGGUGGG | hsa-miR-181b |
| 266 | AACAUUCAUUGUUGUCGGUGGGUU | hsa-miR-181d |
| 267 | GGCAAGAUGCUGGCAUAGCUG | hsa-miR-31 |
| 268 | AACACACCUGGUUAACCUCUUU | hsa-miR-329 |
| 269 | AUCAUAGAGGAAAAUCCAUGUU | hsa-miR-376b |
| 270 | AUCGGGAAUGUCGUGUCCGCC | hsa-miR-425 |
| 271 | AAACCGUUACCAUUACUGAGUUU | hsa-miR-451 |
| 272 | CCCAGAUAAUGGCACUCUCAA | hsa-miR-488 |
| 273 | AGUGACAUCACAUAUACGGCAGC | hsa-miR-489 |
| 274 | AAACAAACAUGGUGCACUUCUUU | hsa-miR-495 |
| 275 | AUCCUUGCUAUCUGGGUGCUA | hsa-miR-502 |
| 276 | AGACCCUGGUCUGCACUCUAU | hsa-miR-504 |
| 277 | GUGUCUUUUGCUCUGCAGUCA | hsa-miR-511 |
| 278 | UUCUCCAAAAGAAAGCACUUUCUG | hsa-miR-515-5p |
| 279 | CCUCUAGAUGGAAGCACUGUCU | hsa-miR-517* |
| 280 | AAAGUGCAUCCUUUUAGAGGUUU | hsa-miR-519b |
| 281 | AAAGUGCUUCCUUUUAGAGGG | hsa-miR-520b |
| 282 | AAAGUGCUUCCUUUUAGAGGGUU | hsa-miR-520c |
| 283 | AAAGUGCUUCUCUUUGGUGGGUU | hsa-miR-520d |
| 284 | AAAGUGCUUCCUUUUUGAGGG | hsa-miR-520e |
| 285 | AAGUGCUUCCUUUUAGAGGGUU | hsa-miR-520f |
| 286 | ACAAAGUGCUUCCCUUUAGAGUGU | hsa-miR-520g |
| 287 | AACGCACUUCCCUUUAGAGUGU | hsa-miR-521 |
| 288 | GAAGGCGCUUCCCUUUAGAGC | hsa-miR-525* |
| 289 | CUCUAGAGGGAAGCACUUUCU | hsa-miR-526a |
| 290 | AAAGUGCUUCCUUUUAGAGGC | hsa-miR-526b* |
| 291 | UACUCAGGAGAGUGGCAAUCACA | hsa-miR-510 |
| 292 | CACUCAGCCUUGAGGGCACUUUC | hsa-miR-512-5p |
| 293 | UUCACAGGGAGGUGUCAUUUAU | hsa-miR-513 |
| 294 | AUUGACACUUCUGUGAGUAG | hsa-miR-514 |
| 295 | GAGUGCCUUCUUUUGGAGCGU | hsa-miR-515-3p |
| 296 | UGCUUCCUUUCAGAGGGU | hsa-miR-516-3p |
| 297 | AUCUGGAGGUAAGAAGCACUUU | hsa-miR-516b |
| 298 | AUCGUGCAUCCCUUUAGAGUGUU | hsa-miR-517a |
| 299 | UCGUGCAUCCCUUUAGAGUGUU | hsa-miR-517b |
| 300 | AUCGUGCAUCCUUUUAGAGUGU | hsa-miR-517c |
| 301 | AAAGCGCUUCCCUUUGCUGGA | hsa-miR-518a |
| 302 | CAAAGCGCUCCCCUUUAGAGGU | hsa-miR-518b |
| 303 | CAAAGCGCUUCUCUUUAGAGUG | hsa-miR-518c |
| 304 | UCUCUGGAGGGAAGCACUUUCUG | hsa-miR-518c* |
| 305 | CAAAGCGCUUCCCUUUGGAGC | hsa-miR-518d |
| 306 | AAAGCGCUUCCCUUCAGAGUGU | hsa-miR-518e |
| 307 | AAAGCGCUUCUCUUUAGAGGA | hsa-miR-518f |
| 308 | AAAGUGCAUCCUUUUAGAGUGUUAC | hsa-miR-519a |
| 309 | AAAGUGCAUCUUUUUAGAGGAU | hsa-miR-519c |
| 310 | CAAAGUGCCUCCCUUUAGAGUGU | hsa-miR-519d |
| 311 | AAAGUGCCUCCUUUUAGAGUGU | hsa-miR-519e |
| 312 | UUCUCCAAAAGGGAGCACUUUC | hsa-miR-519e* |
| 313 | AAAGUGCUUCCCUUUGGACUGU | hsa-miR-520a |
| 314 | CUCCAGAGGGAAGUACUUUCU | hsa-miR-520a* |
| 315 | UCUACAAAGGGAAGCCCUUUCUG | hsa-miR-520d* |
| 316 | ACAAAGUGCUUCCCUUUAGAGU | hsa-miR-520h |
| 317 | AAAAUGGUUCCCUUUAGAGUGUU | hsa-miR-522 |
| 318 | AACGCGCUUCCCUAUAGAGGG | hsa-miR-523 |
| 319 | GAAGGCGCUUCCCUUUGGAGU | hsa-miR-524 |
| 320 | CUCCAGAGGGAUGCACUUUCU | hsa-miR-525 |
| 321 | CUCUUGAGGGAAGCACUUUCUGUU | hsa-miR-526b |
| 322 | CUCUAGAGGGAAGCGCUUUCUGUU | hsa-miR-526c |
| 323 | CUGCAAAGGGAAGCCCUUUCU | hsa-miR-527 |
| 324 | CAGUAGUGAUGAAAUUCCACUUCAUUGGUCCGUGUUU | U18 |
| | CUGAACCACAUGAUUUUCUCGGAUGUUCUGAUG | |
| 325 | CUGCGAUGAUGGCAUUUCUUAGGACACCUUUGGAUUA | RNU58B |
| | AUAAUGAAAACAACUACUCUCUGAGCAGC | |
| 326 | CUGCAGUGAUGACUUUCUUGGGACACCUUUGGAUUUA | RNU58A |
| | CCGUGAAAAUUAAUAAAUUCUGAGCAGC | |
| 327 | CUUAAUGAUGACUGUUUUUUUUGAUUGCUUGAAGCAA | RPL21 |
| | UGUGAAAAACACAUUUCACCGGCUCUGAAAGCU | |
| 328 | UGGCGAUGAGGAGGUACCUAUUGUGUUGAGUAACGGU | U54 |
| | GAUAAUUUUAUACGCUAUUCUGAGCC | |
| 329 | CCAGUCACAGAUUUCUUUGUUCCUUCUCCACUCCCAC | HY3 |
| | UGCAUCACUUAACUAGCCUU | |
| 330 | AGCCUGUGAUGCUUUAAGAGUAGUGGACAGAAGGGAU | U75 |
| | UUCUGAAAUUCUAUUCUGAGGCU | |
| 331 | UAAUGAUUCUGCCAAAUGAAAUAUAAUGAUAUCACUG | U47 |
| | UAAAACCGUUCCAUUUUGAUUCUGAGGU | |
| 332 | AAUUGCACGGUAUCCAUCUGUA | hsa-miR-363 |
| 333 | ACUGCCCUAAGUGCUCCUUCU | hsa-miR-18a* |
| 334 | AAUCCUUGGAACCUAGGUGUGAGU | hsa-miR-362 |
| 335 | AAUAUAACACAGAUGGCCUGU | hsa-miR-410 |
| 336 | UCACUCCUCUCCUCCCGUCUUCU | hsa-miR-483 |
| 337 | GUCAUACACGGCUCUCCUCUCU | hsa-miR-485-3p |
| 338 | UCCUGUACUGAGCUGCCCCGAG | hsa-miR-486 |
| 339 | AAUCAUACAGGGACAUCCAGUU | hsa-miR-487a |
| 340 | UAUGUGCCUUUGGACUACAUCG | hsa-miR-455 |
| 341 | CAUCUGGAGGUAAGAAGCACUUU | hsa-miR-516-5p |
| 342 | UGAAGGUCUACUGUGUGCCAG | hsa-miR-493-3p |
| 343 | CGGGUGGAUCACGAUGCAAUUU | hsa-miR-363* |
| 344 | UGUGACAGAUUGAUAACUGAAA | hsa-miR-542-3p |
| 345 | AAUCGUACAGGGUCAUCCACUU | hsa-miR-487b |
| 346 | GGAGAAAUUAUCCUUGGUGUGU | hsa-miR-539 |
| 347 | GGUAGAUUCUCCUUCUAUGAG | hsa-miR-376a* |
| 348 | UCGGGGAUCAUCAUGUCACGAG | hsa-miR-542-5p |
| 349 | AUCAGCAAACAUUUAUUGUGUG | hsa-miR-545 |
| 350 | AUUCUGCAUUUUUAGCAAGU | hsa-miR-544 |
| 351 | AAUAUUAUACAGUCAACCUCU | hsa-miR-656 |
| 352 | UGACAACUAUGGAUGAGCUCU | hsa-miR-549 |
| 353 | GGCAGGUUCUCACCCUCUCUAGG | hsa-miR-657 |
| 354 | GGCGGAGGGAAGUAGGUCCGUUGGU | hsa-miR-658 |
| 355 | CUUGGUUCAGGGAGGGUCCCCA | hsa-miR-659 |
| 356 | UACCCAUUGCAUAUCGGAGUUG | hsa-miR-660 |
| 357 | AAUGACACGAUCACUCCCGUUGA | hsa-miR-425-5p |
| 358 | AAUGGCGCCACUAGGGUUGUGCA | hsa-miR-652 |
| 359 | CAUGCCUUGAGUGUAGGACCGU | hsa-miR-532 |
| 360 | GCGACCCACUCUUGGUUUCCA | hsa-miR-551a |
| 361 | AACAGGUGACUGGUUAGACAA | hsa-miR-552 |
| 362 | AAAACGGUGAGAUUUUGUUUU | hsa-miR-553 |
| 363 | GCUAGUCCUGACUCAGCCAGU | hsa-miR-554 |
| 364 | AGGGUAAGCUGAACCUCUGAU | hsa-miR-555 |
| 365 | GAUGAGCUCAUUGUAAUAUG | hsa-miR-556 |
| 366 | GUUUGCACGGGUGGGCCUUGUCU | hsa-miR-557 |
| 367 | UGAGCUGCUGUACCAAAAU | hsa-miR-558 |
| 368 | UAAAGUAAAUAUGCACCAAAA | hsa-miR-559 |
| 369 | CAAAGUUUAAGAUCCUUGAAGU | hsa-miR-561 |
| 370 | AAAGUAGCUGUACCAUUUGC | hsa-miR-562 |
| 371 | AGGUUGACAUACGUUUCCC | hsa-miR-563 |
| 372 | AGGCACGGUGUCAGCAGGC | hsa-miR-564 |
| 373 | GGCUGGCUCGCGAUGUCUGUUU | hsa-miR-565 |
| 374 | GGGCGCCUGUGAUCCCAAC | hsa-miR-566 |
| 375 | AGUAUGUUCUUCCAGGACAGAAC | hsa-miR-567 |
| 376 | GCGACCCAUACUUGGUUUCAG | hsa-miR-551b |
| 377 | AGUUAAUGAAUCCUGGAAAGU | hsa-miR-569 |
| 378 | GAAAACAGCAAUUACCUUUGCA | hsa-miR-570 |
| 379 | CAAAACUGGCAAUUACUUUUGC | hsa-miR-548a |
| 380 | UAUGCAUUGUAUUUUUAGGUCC | hsa-miR-586 |
| 381 | UUUCCAUAGGUGAUGAGUCAC | hsa-miR-587 |
| 382 | CAAGAACCUCAGUUGCUUUUGU | hsa-miR-548b |
| 383 | UUGGCCACAAUGGGUUAGAAC | hsa-miR-588 |
| 384 | UCAGAACAAAUGCCGGUUCCCAGA | hsa-miR-589 |
| 385 | UGUCUUACUCCCUCAGGCACAU | hsa-miR-550 |
| 386 | AGACCAUGGGUUCUCAUUGU | hsa-miR-591 |
| 387 | UUGUGUCAAUAUGCGAUGAUGU | hsa-miR-592 |
| 388 | AGGCACCAGCCAGGCAUUGCUCAGC | hsa-miR-593 |
| 389 | CCCAUCUGGGGUGGCCUGUGACUUU | hsa-miR-594 |
| 390 | AAGCCUGCCCGGCUCCUCGGG | hsa-miR-596 |
| 391 | UGUGUCACUCGAUGACCACUGU | hsa-miR-597 |
| 392 | ACAGUCUGCUGAGGUUGGAGC | hsa-miR-622 |
| 393 | GUUGUGUCAGUUUAUCAAAC | hsa-miR-599 |
| 394 | AUCCCUUGCAGGGGCUGUUGGGU | hsa-miR-623 |
| 395 | ACUUACAGACAAGAGCCUUGCUC | hsa-miR-600 |
| 396 | UAGUACCAGUACCUUGUGUUCA | hsa-miR-624 |
| 397 | UGGUCUAGGAUUGUUGGAGGAG | hsa-miR-601 |
| 398 | AGCUGUCUGAAAAUGUCUU | hsa-miR-626 |
| 399 | GUGAGUCUCUAAGAAAAGAGGA | hsa-miR-627 |
| 400 | UCUAGUAAGAGUGGCAGUCG | hsa-miR-628 |
| 401 | GUUCUCCCAACGUAAGCCCAGC | hsa-miR-629 |
| 402 | AGUAUUCUGUACCAGGGAAGGU | hsa-miR-630 |
| 403 | AGACCUGGCCCAGACCUCAGC | hsa-miR-631 |
| 404 | GUGCAUUGCUGUUGCAUUGCA | hsa-miR-33b |
| 405 | CACACACUGCAAUUACUUUUGC | hsa-miR-603 |
| 406 | AGGCUGCGGAAUUCAGGAC | hsa-miR-604 |
| 407 | UAAAUCCCAUGGUGCCUUCUCCU | hsa-miR-605 |
| 408 | AAACUACUGAAAAUCAAAGAU | hsa-miR-606 |
| 409 | GUUCAAAUCCAGAUCUAUAAC | hsa-miR-607 |
| 410 | AGGGGUGGUGUUGGGACAGCUCCGU | hsa-miR-608 |
| 411 | GUGUCUGCUUCCUGUGGGA | hsa-miR-632 |
| 412 | AGGGUGUUUCUCUCAUCUCU | hsa-miR-609 |
| 413 | CUAAUAGUAUCUACCACAAUAAA | hsa-miR-633 |
| 414 | UGAGCUAAAUGUGUGCUGGGA | hsa-miR-610 |
| 415 | AACCAGCACCCCAACUUUGGAC | hsa-miR-634 |
| 416 | ACUUGGGCACUGAAACAAUGUCC | hsa-miR-635 |
| 417 | GCUGGGCAGGGCUUCUGAGCUCCUU | hsa-miR-612 |
| 418 | UGUGCUUGCUCGUCCCGCCCGCAG | hsa-miR-636 |
| 419 | ACUGGGGGCUUUCGGGCUCUGCGU | hsa-miR-637 |
| 420 | AGGGAUCGCGGGCGGGUGGCGGCCU | hsa-miR-638 |
| 421 | AUCGCUGCGGUUGCGAGCGCUGU | hsa-miR-639 |
| 422 | AUGAUCCAGGAACCUGCCUCU | hsa-miR-640 |
| 423 | AAAGACAUAGGAUAGAGUCACCUC | hsa-miR-641 |
| 424 | AGGAAUGUUCCUUCUUUGCC | hsa-miR-613 |
| 425 | GAACGCCUGUUCUUGCCAGGUGG | hsa-miR-614 |
| 426 | UCCGAGCCUGGGUCUCCCUCU | hsa-miR-615 |
| 427 | ACUCAAAACCCUUCAGUGACUU | hsa-miR-616 |
| 428 | CAAAAAUCUCAAUUACUUUUGC | hsa-miR-548c |
| 429 | AGACUUCCCAUUUGAAGGUGGC | hsa-miR-617 |
| 430 | GUCCCUCUCCAAAUGUGUCUUG | hsa-miR-642 |
| 431 | AAACUCUACUUGUCCUUCUGAGU | hsa-miR-618 |
| 432 | ACUUGUAUGCUAGCUCAGGUAG | hsa-miR-643 |
| 433 | GACCUGGACAUGUUUGUGCCCAGU | hsa-miR-619 |
| 434 | AGUGUGGCUUUCUUAGAGC | hsa-miR-644 |
| 435 | UCUAGGCUGGUACUGCUGA | hsa-miR-645 |
| 436 | GGCUAGCAACAGCGCUUACCU | hsa-miR-621 |
| 437 | AAGCAGCUGCCUCUGAGGC | hsa-miR-646 |
| 438 | GUGGCUGCACUCACUUCCUUC | hsa-miR-647 |
| 439 | AAGUGUGCAGGGCACUGGU | hsa-miR-648 |
| 440 | AAACCUGUGUUGUUCAAGAGUC | hsa-miR-649 |
| 441 | AGGAGGCAGCGCUCUCAGGAC | hsa-miR-650 |
| 442 | UUUAGGAUAAGCUUGACUUUUG | hsa-miR-651 |
| 443 | CAAAAACCACAGUUUCUUUUGC | hsa-miR-548d |
| 444 | UGCCUGGGUCUCUGGCCUGCGCGU | hsa-miR-661 |
| 445 | UCCCACGUUGUGGCCCAGCAG | hsa-miR-662 |
| 446 | AGGCAGUGUAUUGUUAGCUGGC | hsa-miR-449b |
| 447 | UUGAAACAAUCUCUACUGAAC | hsa-miR-653 |
| 448 | UAGUAGACCGUAUAGCGUACG | hsa-miR-411 |
| 449 | UGGUGGGCCGCAGAACAUGUGC | hsa-miR-654 |
| 450 | AUAAUACAUGGUUAACCUCUUU | hsa-miR-655 |
| 451 | UGAGUUGGCCAUCUGAGUGAG | hsa-miR-571 |
| 452 | GUCCGCUCGGCGGUGGCCCA | hsa-miR-572 |
| 453 | CUGAAGUGAUGUGUAACUGAUCAG | hsa-miR-573 |
| 454 | GAGCCAGUUGGACAGGAGC | hsa-miR-575 |
| 455 | AUUCUAAUUUCUCCACGUCUUUG | hsa-miR-576 |
| 456 | CUUCUUGUGCUCUAGGAUUGU | hsa-miR-578 |
| 457 | AUUCAUUUGGUAUAAACCGCGAU | hsa-miR-579 |
| 458 | UUGAGAAUGAUGAAUCAUUAGG | hsa-miR-580 |
| 459 | UCUUGUGUUCUCUAGAUCAGU | hsa-miR-581 |
| 460 | CAAAGAGGAAGGUCCCAUUAC | hsa-miR-583 |
| 461 | UUAUGGUUUGCCUGGGACUGAG | hsa-miR-584 |
| 462 | UGGGCGUAUCUGUAUGCUA | hsa-miR-585 |
| 463 | UGGCAGUGAUGAUCACAAAUCCGUGUUUCUGACAAGC | U18 |
| | GAUUGACGAUAGAAAACCGGCUGAGCCA | |
| 464 | UAAUACUGCCUGGUAAUGAUGAC | hsa-miR-200b |
| 465 | UCAGGCUCAGUCCCCUCCCGAU | hsa-miR-484 |
| 466 | AAGUGCUGUCAUAGCUGAGGUC | hsa-miR-512-3p |
| 467 | UGUCUUGCAGGCCGUCAUGCA | hsa-miR-431 |
| 468 | CUACAAAGGGAAGCACUUUCUC | hsa-miR-524-5p |
| 469 | UUACAGUUGUUCAACCAGUUACU | hsa-miR-582-5p |
| 470 | GAGCUUAUUCAUAAAAGUGCAG | hsa-miR-590-5p |
| 471 | ACUCCAGCCCCACAGCCUCAGC | hsa-miR-766 |
| 472 | GAAGUGUGCCGUGGUGUGUCU | hsa-miR-595 |
| 473 | UACGUCAUCGUUGUCAUCGUCA | hsa-miR-598 |
| 474 | UUUGUGACCUGGUCCACUAACC | hsa-miR-758 |
| 475 | UGUCACUCGGCUCGGCCCACUAC | hsa-miR-668 |
| 476 | UGCACCAUGGUUGUCUGAGCAUG | hsa-miR-767-5p |
| 477 | GAUUGCUCUGCGUGCGGAAUCGAC | hsa-miR-801 |
| 478 | UCUGCUCAUACCCCAUGGUUUCU | hsa-miR-767-3p |
| 479 | ACCCUAUCAAUAUUGUCUCUGC | hsa-miR-454* |
| 480 | UGAGACCUCUGGGUUCUGAGCU | hsa-miR-769-5p |
| 481 | GUUGGAGGAUGAAAGUACGGAGUGAU | hsa-miR-768-5p |
| 482 | UCACAAUGCUGACACUCAAACUGCUGAC | hsa-miR-768-3p |
| 483 | UCCAGUACCACGUGUCAGGGCCA | hsa-miR-770-5p |
| 484 | CUGGGAUCUCCGGGGUCUUGGUU | hsa-miR-769-3p |
| 485 | CAGUAACAAAGAUUCAUCCUUGU | hsa-miR-802 |
| 486 | UGGUGCGGAGAGGGCCCACAGUG | hsa-miR-675 |
| 487 | GCACUGAGAUGGGAGUGGUGUA | hsa-miR-674 |
| 488 | AAUGCACCUGGGCAAGGAUUCA | hsa-miR-502-3p |
| 489 | AGACCCUGGUCUGCACUCUAUC | hsa-miR-504 |
| 490 | GUGCAUUGCUGUUGCAUUGC | hsa-miR-33b |
| 491 | GGGAGCCAGGAAGUAUUGAUGU | hsa-miR-505* |
| 492 | UGUGCUUGCUCGUCCCGCCCGCA | hsa-miR-636 |
| 493 | CGUCAACACUUGCUGGUUUCCU | hsa-miR-505 |
| 494 | UUCACAGGGAGGUGUCAU | hsa-miR-513-5p |
| 495 | UAAAUUUCACCUUUCUGAGAAGG | hsa-miR-513-3p |
| 496 | UACUCCAGAGGGCGUCACUCAUG | hsa-miR-508-5p |
| 497 | CGGGGCAGCUCAGUACAGGAU | hsa-miR-486-3p |
| 498 | AUGGUUCCGUCAAGCACCAUGG | hsa-miR-218-1* |
| 499 | AGAGUUGAGUCUGGACGUCCCG | hsa-miR-219-1-3p |
| 500 | ACCUGGCAUACAAUGUAGAUUU | hsa-miR-221* |
| 501 | CUCAGUAGCCAGUGUAGAUCCU | hsa-miR-222* |
| 502 | CGUGUAUUUGACAAGCUGAGUU | hsa-miR-223* |
| 503 | CAAGUCACUAGUGGUUCCGUU | hsa-miR-224 |
| 504 | CAUCAUCGUCUCAAAUGAGUCU | hsa-miR-136* |
| 505 | GAGGGUUGGGUGGAGGCUCUCC | hsa-miR-296-3p |
| 506 | CAAUCACUAACUCCACUGCCAU | hsa-miR-34b |
| 507 | AGGGGCUGGCUUUCCUCUGGUC | hsa-miR-185* |
| 508 | GCCCAAAGGUGAAUUUUUUGGG | hsa-miR-186* |
| 509 | CUCCCACAUGCAGGGUUUGCA | hsa-miR-188-3p |
| 510 | CCAAUAUUGGCUGUGCUGCUCC | hsa-miR-195* |
| 511 | CUGGGAGAGGGUUGUUUACUCC | hsa-miR-30c-1* |
| 512 | UAUUGCACAUUACUAAGUUGCA | hsa-miR-32 |
| 513 | CUGGGAGAAGGCUGUUUACUCU | hsa-miR-30c-2* |
| 514 | CAAUUUAGUGUGUGUGAUAUUU | hsa-miR-32* |
| 515 | UAGCACCAUCUGAAAUCGGUUA | hsa-miR-29a |
| 516 | UGCUAUGCCAACAUAUUGCCAU | hsa-miR-31* |
| 517 | ACUCUUUCCCUGUUGCACUAC | hsa-miR-130b* |
| 518 | CCUAUUCUUGAUUACUUGUUUC | hsa-miR-26a-2* |
| 519 | UCCCCCAGGUGUGAUUCUGAUUU | hsa-miR-361-3p |
| 520 | AACACACCUAUUCAAGGAUUCA | hsa-miR-362-3p |
| 521 | CUGUACAGGCCACUGCCUUGC | hsa-let-7g* |
| 522 | ACUUUAACAUGGAAGUGCUUUC | hsa-miR-302b* |
| 523 | ACUUUAACAUGGAGGCACUUGC | hsa-miR-302d* |
| 524 | ACUGUUGCUAAUAUGCAACUCU | hsa-miR-367* |
| 525 | AACAUAGAGGAAAUUCCACGU | hsa-miR-376c |
| 526 | AAGUGCCGCCAUCUUUUGAGUGU | hsa-miR-371-3p |
| 527 | CUUAUCAGAUUGUAUUGUAAUU | hsa-miR-374a* |
| 528 | UGGGUUCCUGGCAUGCUGAUUU | hsa-miR-23b* |
| 529 | GUAGAUUCUCCUUCUAUGAGUA | hsa-miR-376a* |
| 530 | AGAGGUUGCCCUUGGUGAAUUC | hsa-miR-377* |
| 531 | CUGGGAGGUGGAUGUUUACUUC | hsa-miR-30b* |
| 532 | AACGCCAUUAUCACACUAAAUA | hsa-miR-122* |
| 533 | UUCACAUUGUGCUACUGUCUGC | hsa-miR-130a* |
| 534 | ACCGUGGCUUUCGAUUGUUACU | hsa-miR-132* |
| 535 | UAUGUAACAUGGUCCACUAACU | hsa-miR-379* |
| 536 | AAAGUUCUGAGACACUCCGACU | hsa-miR-148a* |
| 537 | GUGCAUUGUAGUUGCAUUGCA | hsa-miR-33a |
| 538 | CAAUGUUUCCACAGUGCAUCAC | hsa-miR-33a* |
| 539 | AGGUUGGGAUCGGUUGCAAUGCU | hsa-miR-92a-1* |
| 540 | GGGUGGGGAUUUGUUGCAUUAC | hsa-miR-92a-2* |
| 541 | ACUGCUGAGCUAGCACUUCCCG | hsa-miR-93* |
| 542 | AAUCAUGUGCAGUGCCAAUAUG | hsa-miR-96* |
| 543 | CAAGCUCGCUUCUAUGGGUCUG | hsa-miR-99a* |
| 544 | CAAGCUUGUAUCUAUAGGUAUG | hsa-miR-100* |
| 545 | CAGUUAUCACAGUGCUGAUGCU | hsa-miR-101* |
| 546 | GCUAUUUCACGACACCAGGGUU | hsa-miR-138-2* |
| 547 | CAUCUUCCAGUACAGUGUUGGA | hsa-miR-141* |
| 548 | GGUGCAGUGCUGCAUCUCUGGU | hsa-miR-143* |
| 549 | AGGGGUGCUAUCUGUGAUUGA | hsa-miR-342-5p |
| 550 | GGAUAUCAUCAUAUACUGUAAG | hsa-miR-144* |
| 551 | GGAUUCCUGGAAAUACUGUUCU | hsa-miR-145* |
| 552 | GGGGAGCUGUGGAAGCAGUA | hsa-miR-920 |
| 553 | CUAGUGAGGGACAGAACCAGGAUUC | hsa-miR-921 |
| 554 | GCAGCAGAGAAUAGGACUACGUC | hsa-miR-922 |
| 555 | GUCAGCGGAGGAAAAGAAACU | hsa-miR-923 |
| 556 | AGAGUCUUGUGAUGUCUUGC | hsa-miR-924 |
| 557 | UACUGCAGACGUGGCAAUCAUG | hsa-miR-509-3-5p |
| 558 | GAACGGCUUCAUACAGGAGUU | hsa-miR-337-5p |
| 559 | CUCCUAUAUGAUGCCUUUCUUC | hsa-miR-337-3p |
| 560 | UCACAAGUCAGGCUCUUGGGAC | hsa-miR-125b-2* |
| 561 | AUGUAGGGCUAAAAGCCAUGGG | hsa-miR-135b* |
| 562 | AAGUUCUGUUAUACACUCAGGC | hsa-miR-148b* |
| 563 | ACUGCCCCAGGUGCUGCUGG | hsa-miR-324-3p |
| 564 | GCUACUUCACAACACCAGGGCC | hsa-miR-138-1* |
| 565 | CCUCUGAAAUUCAGUUCUUCAG | hsa-miR-146a* |
| 566 | AGGGAGGGACGGGGGCUGUGC | hsa-miR-149* |
| 567 | GCUGGUUUCAUAUGGUGGUUUAGA | hsa-miR-29b-1* |
| 568 | CUGGUUUCACAUGGUGGCUUAG | hsa-miR-29b-2* |
| 569 | UCAAAUGCUCAGACUCCUGUGGU | hsa-miR-105 |
| 570 | ACGGAUGUUUGAGCAUGUGCUA | hsa-miR-105* |
| 571 | AAAAGUGCUUACAGUGCAGGUAG | hsa-miR-106a |
| 572 | CUGCAAUGUAAGCACUUCUUAC | hsa-miR-106a* |
| 573 | CCAAUAUUACUGUGCUGCUUUA | hsa-miR-16-2* |
| 574 | CUGCGCAAGCUACUGCCUUGCU | hsa-let-7i* |
| 575 | CGAAUCAUUAUUUGCUGCUCUA | hsa-miR-15b* |
| 576 | AGAGCUUAGCUGAUUGGUGAAC | hsa-miR-27b* |
| 577 | UGUGCGCAGGGAGACCUCUCCC | hsa-miR-933 |
| 578 | UGUCUACUACUGGAGACACUGG | hsa-miR-934 |
| 579 | CCAGUUACCGCUUCCGCUACCGC | hsa-miR-935 |
| 580 | ACAGUAGAGGGAGGAAUCGCAG | hsa-miR-936 |
| 581 | AUCCGCGCUCUGACUCUCUGCC | hsa-miR-937 |
| 582 | UGCCCUUAAAGGUGAACCCAGU | hsa-miR-938 |
| 583 | UGGGGAGCUGAGGCUCUGGGGGUG | hsa-miR-939 |
| 584 | CACCCGGCUGUGUGCACAUGUGC | hsa-miR-941 |
| 585 | UGAGCGCCUCGACGACAGAGCCG | hsa-miR-339-3p |
| 586 | UUUUUCAUUAUUGCUCCUGACC | hsa-miR-335* |
| 587 | GCUGACUCCUAGUCCAGGGCUC | hsa-miR-345 |
| 588 | UCUUCUCUGUUUUGGCCAUGUG | hsa-miR-942 |
| 589 | CUGACUGUUGCCGUCCUCCAG | hsa-miR-943 |
| 590 | AAAUUAUUGUACAUCGGAUGAG | hsa-miR-944 |
| 591 | AGCAGAAGCAGGGAGGUUCUCCCA | hsa-miR-298 |
| 592 | UGCAACGAACCUGAGCCACUGA | hsa-miR-891a |
| 593 | CGGGUCGGAGUUAGCUCAAGCGG | hsa-miR-886-5p |
| 594 | CGCGGGUGCUUACUGACCCUU | hsa-miR-886-3p |
| 595 | CACUGUGUCCUUUCUGCGUAG | hsa-miR-892a |
| 596 | CAAGCUCGUGUCUGUGGGUCCG | hsa-miR-99b* |
| 597 | CGUGUUCACAGCGGACCUUGAU | hsa-miR-124* |
| 598 | UCCCUGAGACCCUUUAACCUGUGA | hsa-miR-125a-5p |
| 599 | ACAGGUGAGGUUCUUGGGAGCC | hsa-miR-125 a-3p |
| 600 | AAAGGAUUCUGCUGUCGGUCCCACU | hsa-miR-541* |
| 601 | UGGUGGGCACAGAAUCUGGACU | hsa-miR-541 |
| 602 | UUAAUAUCGGACAACCAUUGU | hsa-miR-889 |
| 603 | UAUACCUCAGUUUUAUCAGGUG | hsa-miR-875-5p |
| 604 | CCUGGAAACACUGAGGUUGUG | hsa-miR-875-3p |
| 605 | UGGAUUUCUUUGUGAAUCACCA | hsa-miR-876-5p |
| 606 | CCACCACCGUGUCUGACACUU | hsa-miR-220b |
| 607 | UUUUGCAAUAUGUUCCUGAAUA | hsa-miR-450b-5p |
| 608 | UUGGGAUCAUUUUGCAUCCAUA | hsa-miR-450b-3p |
| 609 | UACUUGGAAAGGCAUCAGUUG | hsa-miR-890 |
| 610 | UGCAACUUACCUGAGUCAUUGA | hsa-miR-891b |
| 611 | ACACAGGGCUGUUGUGAAGACU | hsa-miR-220c |
| 612 | UACUCAAAAAGCUGUCAGUCA | hsa-miR-888 |
| 613 | GACUGACACCUCUUUGGGUGAA | hsa-miR-888* |
| 614 | CACUGGCUCCUUUCUGGGUAGA | hsa-miR-892b |
| 615 | UAGGUAGUUUCCUGUUGUUGGG | hsa-miR-196b |
| 616 | UCACAGUGAACCGGUCUCUUU | hsa-miR-128a |
| 617 | UAAGGUGCAUCUAGUGCAGUUAG | hsa-miR-18b |
| 618 | UACCCUGUAGAACCGAAUUUGUG | hsa-miR-10b |
| 619 | UAAUCUCAGCUGGCAACUGUGA | hsa-miR-216a |
| 620 | UGAGGUAGUAGUUUGUGCUGUU | hsa-let-7i |
| 621 | UGGAAUGUAAAGAAGUAUGUAU | hsa-miR-1 |
| 622 | UGUAAACAUCCUUGACUGGAAG | hsa-miR-30e |
| 623 | UGGUGGUUUACAAAGUAAUUCA | hsa-miR-876-3p |
| 624 | CACAUUACACGGUCGACCUCU | hsa-miR-323-3p |
| 625 | UCGUACCGUGAGUAAUAAUGCG | hsa-miR-126 |
| 626 | CUGAAGCUCAGAGGGCUCUGAU | hsa-miR-127-5p |
| 627 | UCUCUGGGCCUGUGUCUUAGGC | hsa-miR-330-5p |
| 628 | AUAAAGCUAGAUAACCGAAAGU | hsa-miR-9* |
| 629 | UAUAGGGAUUGGAGCCGUGGCG | hsa-miR-135a* |
| 630 | CUAGGUAUGGUCCCAGGGAUCC | hsa-miR-331-5p |
| 631 | UACCACAGGGUAGAACCACGG | hsa-miR-140-3p |
| 632 | UACUGCAGACAGUGGCAAUCA | hsa-miR-509-5p |
| 633 | UGAUUGGUACGUCUGUGGGUAG | hsa-miR-509-3p |
| 634 | AAAAGUAAUUGUGGUUUUUGCC | hsa-miR-548d-5p |
| 635 | UAUGUAACACGGUCCACUAACC | hsa-miR-411* |
| 636 | UAUGUCUGCUGACCAUCACCUU | hsa-miR-654-3p |
| 637 | UCGGGGAUCAUCAUGUCACGAGA | hsa-miR-542-5p |
| 638 | UACUCAGGAGAGUGGCAAUCAC | hsa-miR-510 |
| 639 | ACUGGACUUGGAGUCAGAAGG | hsa-miR-378 |
| 640 | GCAGUCCAUGGGCAUAUACAC | hsa-miR-455-3p |
| 641 | UGGAGUGUGACAAUGGUGUUUG | hsa-miR-122 |
| 642 | UUUGGUCCCCUUCAACCAGCUG | hsa-miR-133a |
| 643 | UUUGGUCCCCUUCAACCAGCUA | hsa-miR-133b |
| 644 | CAUAAAGUAGAAAGCACUACU | hsa-miR-142-5p |
| 645 | UGAGAUGAAGCACUGUAGCUC | hsa-miR-143 |
| 646 | AACUGGCCUACAAAGUCCCAGU | hsa-miR-193a-3p |
| 647 | UAAUACUGCCUGGUAAUGAUGA | hsa-miR-200b |
| 648 | UCCAGCAUCAGUGAUUUUGUUG | hsa-miR-338-3p |
| 649 | UACAGUACUGUGAUAACUGAA | hsa-miR-101 |
| 650 | CUAGACUGAAGCUCCUUGAGG | hsa-miR-151-3p |
| 651 | UCUGGCUCCGUGUCUUCACUCCC | hsa-miR-149 |
| 652 | UCCCUGUCCUCCAGGAGCUCACG | hsa-miR-339-5p |
| 653 | UUAUAAAGCAAUGAGACUGAUU | hsa-miR-340 |
| 654 | UCCGUCUCAGUUACUUUAUAGC | hsa-miR-340* |
| 655 | UCUCACACAGAAAUCGCACCCGU | hsa-miR-342-3p |
| 656 | UAUGGCUUUUCAUUCCUAUGUGA | hsa-miR-135b |
| 657 | GUGUGCGGAAAUGCUUCUGCUA | hsa-miR-147b |
| 658 | UGAUAUGUUUGAUAUUGGGUU | hsa-miR-190b |
| 659 | AAGGUUACUUGUUAGUUCAGG | hsa-miR-872 |
| 660 | AUUCUGCAUUUUUAGCAAGUUC | hsa-miR-544 |
| 661 | UCAGUAAAUGUUUAUUAGAUGA | hsa-miR-545* |
| 662 | UCAGCAAACAUUUAUUGUGUGC | hsa-miR-545 |
| 663 | CUGCCCUGGCCCGAGGGACCGA | hsa-miR-874 |
| 664 | UAUGGCACUGGUAGAAUUCACU | hsa-miR-183 |
| 665 | GUGAAUUACCGAAGGGCCAUAA | hsa-miR-183* |
| 666 | UGGAGAGAAAGGCAGUUCCUGA | hsa-miR-185 |
| 667 | CUGCCAAUUCCAUAGGUCACAG | hsa-miR-192* |
| 668 | GGUCCAGAGGGGAGAUAGGUUC | hsa-miR-198 |
| 669 | CAUCUUACUGGGCAGCAUUGGA | hsa-miR-200b* |
| 670 | GCCUGCUGGGGUGGAACCUGGU | hsa-miR-370 |
| 671 | AGCUACAUCUGGCUACUGGGU | hsa-miR-222 |
| 672 | AAAAGCUGGGUUGAGAGGGCGA | hsa-miR-320 |
| 673 | GUCCAGUUUUCCCAGGAAUCCCU | hsa-miR-145 |
| 674 | AGGCAAGAUGCUGGCAUAGCU | hsa-miR-31 |
| 675 | UGGGUCUUUGCGGGCGAGAUGA | hsa-miR-193a-5p |
| 676 | UGAGGUAGUAGUUUGUACAGUU | hsa-let-7g |
| 677 | AGAGGUAGUAGGUUGCAUAGUU | hsa-let-7d |
| 678 | AGCUGGUGUUGUGAAUCAGGCCG | hsa-miR-138 |
| 679 | CAAAGAAUUCUCCUUUUGGGCU | hsa-miR-186 |
| 680 | CGUCUUACCCAGCAGUGUUUGG | hsa-miR-200c* |
| 681 | CUCCUACAUAUUAGCAUUAACA | hsa-miR-155* |
| 682 | CAAAUUCGUAUCUAGGGGAAUA | hsa-miR-10a* |
| 683 | UCUACAGUGCACGUGUCUCCAG | hsa-miR-139-5p |
| 684 | AUAAGACGAACAAAAGGUUUGU | hsa-miR-208b |
| 685 | GUGUUGAAACAAUCUCUACUG | hsa-miR-653 |
| 686 | UGCCUGUCUACACUUGCUGUGC | hsa-miR-214* |
| 687 | CAUGGUUCUGUCAAGCACCGCG | hsa-miR-218-2* |
| 688 | UGUCAGUUUGUCAAAUACCCCA | hsa-miR-223 |
| 689 | UCCAUUACACUACCCUGCCUCU | hsa-miR-885-5p |
| 690 | ACUGGACUUAGGGUCAGAAGGC | hsa-miR-422a |
| 691 | AAGCCCUUACCCCAAAAAGUAU | hsa-miR-129* |
| 692 | CAACGGAAUCCCAAAAGCAGCUG | hsa-miR-191 |
| 693 | UAAUACUGCCGGGUAAUGAUGGA | hsa-miR-200c |
| 694 | AGUUCUUCAGUGGCAAGCUUUA | hsa-miR-22* |
| 695 | AUCGGGAAUGUCGUGUCCGCCC | hsa-miR-425* |
| 696 | UUUUGCGAUGUGUUCCUAAUAU | hsa-miR-450a |
| 697 | ACAGUAGUCUGCACAUUGGUUA | hsa-miR-199a-3p |
| 698 | CUUUCAGUCAGAUGUUUGCUGC | hsa-miR-30d* |
| 699 | ACAGCAGGCACAGACAGGCAGU | hsa-miR-214 |
| 700 | CUAUACAAUCUACUGUCUUUC | hsa-let-7a* |
| 10 | CAAAGUGCUUACAGUGCAGGUAG | hsa-miR-17 |
| 701 | CAAAACGUGAGGCGCUGCUAU | hsa-miR-424* |
| 702 | UGCCCUAAAUGCCCCUUCUGGC | hsa-miR-18b* |
| 703 | ACUGUAGUAUGGGCACUUCCAG | hsa-miR-20b* |
| 704 | CAGGUCGUCUUGCAGGGCUUCU | hsa-miR-431* |
| 705 | GGAGACGCGGCCCUGUUGGAGU | hsa-miR-139-3p |
| 706 | CAACAAAUCCCAGUCUACCUAA | hsa-miR-7-2* |
| 707 | ACAGAUUCGAUUCUAGGGGAAU | hsa-miR-10b* |
| 708 | CAAUCAGCAAGUAUACUGCCCU | hsa-miR-34a* |
| 709 | ACCACUGACCGUUGACUGUACC | hsa-miR-181a-2* |
| 710 | AGGUUGUCCGUGGUGAGUUCGCA | hsa-miR-453 |
| 711 | CAUCCCUUGCAUGGUGGAGGG | hsa-miR-188-5p |
| 712 | UCCGGUUCUCAGGGCUCCACC | hsa-miR-671-3p |
| 713 | UAGUGCAAUAUUGCUUAUAGGGU | hsa-miR-454 |
| 714 | UGCGGGGCUAGGGCUAACAGCA | hsa-miR-744 |
| 715 | CUGUUGCCACUAACCUCAACCU | hsa-miR-744* |
| 716 | AAAUCUCUGCAGGCAAAUGUGA | hsa-miR-216b |
| 717 | UGAGGUUGGUGUACUGUGUGUGA | hsa-miR-672 |
| 718 | CGGCUCUGGGUCUGUGGGGA | hsa-miR-760 |
| 719 | AACUGUUUGCAGAGGAAACUGA | hsa-miR-452 |
| 720 | CUCAUCUGCAAAGAAGUAAGUG | hsa-miR-452* |
| 721 | AGGUUACCCGAGCAACUUUGCAU | hsa-miR-409-5p |
| 722 | GAAUGUUGCUCGGUGAACCCCU | hsa-miR-409-3p |
| 723 | AACCAUCGACCGUUGAGUGGAC | hsa-miR-181c* |
| 724 | UUUGGCAAUGGUAGAACUCACACU | hsa-miR-182 |
| 725 | CGGCAACAAGAAACUGCCUGAG | hsa-miR-196a* |
| 726 | UACUGCAUCAGGAACUGAUUGGA | hsa-miR-217 |
| 727 | AAGACGGGAGGAAAGAAGGGAG | hsa-miR-483-5p |
| 728 | UCACUCCUCUCCUCCCGUCUU | hsa-miR-483-3p |
| 729 | UGAGGGGCAGAGAGCGAGACUUU | hsa-miR-423-5p |
| 730 | AAGGAGCUUACAAUCUAGCUGGG | hsa-miR-708 |
| 731 | CAACUAGACUGUGAGCUUCUAG | hsa-miR-708* |
| 732 | AGGGACGGGACGCGGUGCAGUG | hsa-miR-92b* |
| 733 | GAUGAGCUCAUUGUAAUAUGAG | hsa-miR-556-5p |
| 734 | AUAUUACCAUUAGCUCAUCUUU | hsa-miR-556-3p |
| 735 | GAAAUCAAGCGUGGGUGAGACC | hsa-miR-551b* |
| 736 | CGAAAACAGCAAUUACCUUUGC | hsa-miR-570 |
| 737 | CACGCUCAUGCACACACCCACA | hsa-miR-574-3p |
| 738 | AUUCUAAUUUCUCCACGUCUUU | hsa-miR-576-5p |
| 739 | AAGAUGUGGAAAAAUUGGAAUC | hsa-miR-576-3p |
| 740 | AAUGGCGCCACUAGGGUUGUG | hsa-miR-652 |
| 741 | GGGGGUCCCCGGUGCUCGGAUC | hsa-miR-615-5p |
| 742 | UAUUCAGAUUAGUGCCAGUCAUG | hsa-miR-871 |
| 743 | CCUCCCACACCCAAGGCUUGCA | hsa-miR-532-3p |
| 744 | GCAGGAACUUGUGAGUCUCCU | hsa-miR-873 |
| 745 | UUGAAAGGCUAUUUCUUGGUC | hsa-miR-488 |
| 746 | GUGACAUCACAUAUACGGCAGC | hsa-miR-489 |
| 747 | CUUAUGCAAGAUUCCCUUCUAC | hsa-miR-491-3p |
| 748 | UGCCCUGUGGACUCAGUUCUGG | hsa-miR-146b-3p |
| 749 | UUCCUAUGCAUAUACUUCUUUG | hsa-miR-202* |
| 750 | AGAGGUAUAGGGCAUGGGAA | hsa-miR-202 |
| 751 | UGAAGGUCUACUGUGUGCCAGG | hsa-miR-493 |
| 752 | UGAAACAUACACGGGAAACCUC | hsa-miR-494 |
| 753 | CGGGGUUUUGAGGGCGAGAUGA | hsa-miR-193b* |
| 754 | AACUGGCCCUCAAAGUCCCGCU | hsa-miR-193b |
| 755 | CAAACCACACUGUGGUGUUAGA | hsa-miR-497* |
| 756 | GAGUGCCUUCUUUUGGAGCGUU | hsa-miR-515-3p |
| 757 | AAGUGCCUCCUUUUAGAGUGUU | hsa-miR-519e |
| 758 | CUCUAGAGGGAAGCGCUUUCUG | hsa-miR-518e* |
| 759 | AGGCAGCGGGGUGUAGUGGAUA | hsa-miR-885-3p |
| 760 | GUGAACGGGCGCCAUCCCGAGG | hsa-miR-887 |
| 761 | AAACAUUCGCGGUGCACUUCUU | hsa-miR-543 |
| 762 | ACGGGUUAGGCUCUUGGGAGCU | hsa-miR-125b-1* |
| 763 | CCAGUGGGGCUGCUGUUAUCUG | hsa-miR-194* |
| 764 | CCGCACUGUGGGUACUUGCUGC | hsa-miR-106b* |
| 765 | ACUUAAACGUGGAUGUACUUGCU | hsa-miR-302a* |
| 766 | CUCUUGAGGGAAGCACUUUCUGU | hsa-miR-526b |
| 767 | GAAAGUGCUUCCUUUUAGAGGC | hsa-miR-526b* |
| 768 | AAAGUGCAUCCUUUUAGAGGUU | hsa-miR-519b-3p |
| 769 | GAAGGCGCUUCCCUUUAGAGCG | hsa-miR-525-3p |
| 770 | GAACGCGCUUCCCUAUAGAGGGU | hsa-miR-523 |
| 771 | CUCUAGAGGGAAGCACUUUCUC | hsa-miR-518f* |
| 772 | GAAAGCGCUUCUCUUUAGAGG | hsa-miR-518f |
| 773 | CUCUAGAGGGAAGCACUUUCUG | hsa-miR-518d-5p |
| 774 | AGAAUUGUGGCUGGACAUCUGU | hsa-miR-219-2-3p |
| 775 | CUUAGCAGGUUGUAUUAUCAUU | hsa-miR-374b* |
| 776 | CAGUGCAAUGAUAUUGUCAAAGC | hsa-miR-301b |
| 777 | CUACAAAGGGAAGCCCUUUC | hsa-miR-520d-5p |
| 778 | AAAGCGCUUCCCUUCAGAGUG | hsa-miR-518e |
| 779 | CUGCAAAGGGAAGCCCUUUC | hsa-miR-518a-5p |
| 780 | GAAAGCGCUUCCCUUUGCUGGA | hsa-miR-518a-3p |
| 781 | UUCAUUUGGUAUAAACCGCGAUU | hsa-miR-579 |
| 782 | UAACUGGUUGAACAACUGAACC | hsa-miR-582-3p |
| 783 | AAAGUGCUUCCUUUUAGAGGGU | hsa-miR-520c-3p |
| 784 | CAAAGCGCUUCUCUUUAGAGUGU | hsa-miR-518c |
| 785 | AUCGUGCAUCCCUUUAGAGUGU | hsa-miR-517a |
| 786 | CAAAGUGCCUCCCUUUAGAGUG | hsa-miR-519d |
| 787 | CUAUACAACCUACUGCCUUCCC | hsa-let-7b* |
| 788 | UAGAGUUACACCCUGGGAGUUA | hsa-let-7c* |
| 789 | UGAGGUAGGAGGUUGUAUAGUU | hsa-let-7e |
| 790 | CUAUACGGCCUCCUAGCUUUCC | hsa-let-7e* |
| 791 | AAAAGUAAUUGUGGUUUUGGCC | hsa-miR-548b-5p |
| 792 | UGAGAACCACGUCUGCUCUGAG | hsa-miR-589 |
| 793 | AGUGCCUGAGGGAGUAAGAGCCC | hsa-miR-550 |
| 794 | UGUCUCUGCUGGGGUUUCU | hsa-miR-593 |
| 795 | AAAAGUAAUUGCGAGUUUUACC | hsa-miR-548a-5p |
| 796 | AAAAUGGUUCCCUUUAGAGUGU | hsa-miR-522 |
| 797 | AGUCAUUGGAGGGUUUGAGCAG | hsa-miR-616 |
| 798 | AAAGUGCAUCCUUUUAGAGUGU | hsa-miR-519a |
| 799 | UUCUCGAGGAAAGAAGCACUUUC | hsa-miR-516a-5p |
| 800 | CUAUACAAUCUAUUGCCUUCCC | hsa-let-7f-1* |
| 801 | CUAUACAGUCUACUGUCUUUCC | hsa-let-7f-2* |
| 802 | CAGGCCAUAUUGUGCUGCCUCA | hsa-miR-15a* |
| 803 | CCAGUAUUAACUGUGCUGCUGA | hsa-miR-16-1* |
| 804 | ACUGCAGUGAAGGCACUUGUAG | hsa-miR-17* |
| 9 | UAAGGUGCAUCUAGUGCAGAUAG | hsa-miR-18a |
| 805 | ACUGCCCUAAGUGCUCCUUCUGG | hsa-miR-18a* |
| 806 | AGUUUUGCAUAGUUGCACUACA | hsa-miR-19a* |
| 807 | AGUUUUGCAGGUUUGCAUCCAGC | hsa-miR-19b-1* |
| 808 | AGUUUUGCAGGUUUGCAUUUCA | hsa-miR-19b-2* |
| 809 | AACAUCACAGCAAGUCUGUGCU | hsa-miR-499-3p |
| 810 | UAAUCCUUGCUACCUGGGUGAGA | hsa-miR-500 |
| 811 | AAAAGUAAUUGCGGUUUUUGCC | hsa-miR-548c-5p |
| 812 | CACAAGGUAUUGGUAUUACCU | hsa-miR-624 |
| 813 | AGGGGGAAAGUUCUAUAGUCC | hsa-miR-625 |
| 814 | GACUAUAGAACUUUCCCCCUCA | hsa-miR-625* |
| 815 | AUGCUGACAUAUUUACUAGAGG | hsa-miR-628-5p |
| 816 | UCUAGUAAGAGUGGCAGUCGA | hsa-miR-628-3p |
| 817 | AAUGCACCCGGGCAAGGAUUCU | hsa-miR-501-3p |
| 818 | UGGGUUUACGUUGGGAGAACU | hsa-miR-629 |
| 819 | ACUGCAUUAUGAGCACUUAAAG | hsa-miR-20a* |
| 820 | CAACACCAGUCGAUGGGCUGU | hsa-miR-21* |
| 821 | GGGGUUCCUGGGGAUGGGAUUU | hsa-miR-23a* |
| 822 | UGCCUACUGAGCUGAUAUCAGU | hsa-miR-24-1* |
| 823 | UGCCUACUGAGCUGAAACACAG | hsa-miR-24-2* |
| 824 | AGGCGGAGACUUGGGCAAUUG | hsa-miR-25* |
| 825 | CCUAUUCUUGGUUACUUGCACG | hsa-miR-26a-1* |
| 826 | CCUGUUCUCCAUUACUUGGCUC | hsa-miR-26b* |
| 827 | AGGGCUUAGCUGCUUGUGAGCA | hsa-miR-27a* |
| 828 | CACUAGAUUGUGAGCUCCUGGA | hsa-miR-28-3p |
| 829 | ACUGAUUUCUUUUGGUGUUCAG | hsa-miR-29a* |
| 830 | UGAGGUAGUAGGUUGUGUGGUU | hsa-let-7b |
| 831 | UUAAUGCUAAUCGUGAUAGGGGU | hsa-miR-155 |
| 832 | AGCUCGGUCUGAGGCCCCUCAGU | hsa-miR-423-3P |
| 833 | CUGGUACAGGCCUGGGGGACAG | hsa-miR-150* |
| 834 | UCGAGGAGCUCACAGUCUAGU | hsa-miR-151-5P |
| 835 | UGGAGGAGAAGGAAGGUGAUG | hsa-miR-765 |
| 836 | AACAAUAUCCUGGUGCUGAGUG | hsa-miR-338-5P |
| 837 | AUGGAGAUAGAUAUAGAAAU | hsa-miR-620 |
| 838 | UAGAUAAAAUAUUGGUACCUG | hsa-miR-577 |
| 839 | UACAGUAUAGAUGAUGUACU | hsa-miR-144 |
| 840 | UAAUUUUAUGUAUAAGCUAGU | hsa-miR-590-3P |
| 841 | GCUGCGCUUGGAUUUCGUCCCC | hsa-miR-191* |
| 842 | ACCAGGAGGCUGAGGCCCCU | hsa-miR-665 |
| 843 | AGGUGGUCCGUGGCGCGUUCGC | hsa-miR-323-5P |
| 844 | GGCUACAACACAGGACCCGGGC | hsa-miR-187* |
| 845 | AAAGUGCUUCUCUUUGGUGGGU | hsa-miR-520D-3P |
| 846 | CCCCACCUCCUCUCUCCUCAG | hsa-miR-1224-3P |
| 847 | UCCUCUUCUCCCUCCUCCCAG | hsa-miR-877* |
| 848 | UUCUCAAGGAGGUGUCGUUUAU | hsa-miR-513C |
| 849 | UUCACAAGGAGGUGUCAUUUAU | hsa-miR-513B |
| 850 | GUGAGGGCAUGCAGGCCUGGAUGGGG | hsa-miR-1226* |
| 851 | CCUCUUCCCCUUGUCUCUCCAG | hsa-miR-1236 |
| 852 | GUGUCUGGGCGGACAGCUGC | hsa-miR-1231 |
| 853 | GUGGGCGGGGGCAGGUGUGUG | hsa-miR-1228* |
| 854 | GUGGGUACGGCCCAGUGGGGGG | hsa-miR-1225-5P |
| 855 | UCCUUCUGCUCCGUCCCCCAG | hsa-miR-1237 |
| 856 | UGAGCCCCUGUGCCGCCCCCAG | hsa-miR-1225-3P |
| 857 | UGAGCCCUGUCCUCCCGCAG | hsa-miR-1233 |
| 858 | CGUGCCACCCUUUUCCCCAG | hsa-miR-1227 |
| 859 | UGCAGGACCAAGAUGAGCCCU | hsa-miR-1286 |
| 860 | CAAAGGUAUUUGUGGUUUUUG | hsa-miR-548M |
| 861 | AAGCAUUCUUUCAUUGGUUGG | hsa-miR-1179 |
| 862 | UUGCUCACUGUUCUUCCCUAG | hsa-miR-1178 |
| 863 | UCUGCAGGGUUUGCUUUGAG | hsa-miR-1205 |
| 864 | CUUGGCACCUAGCAAGCACUCA | hsa-miR-1271 |
| 865 | AGCCUGAUUAAACACAUGCUCUGA | hsa-miR-1201 |
| 866 | GGGCGACAAAGCAAGACUCUUUCUU | hsa-miR-1273 |
| 867 | AAAAGUAAUUGCGGUCUUUGGU | hsa-miR-548J |
| 868 | AUGGUACCCUGGCAUACUGAGU | hsa-miR-1263 |
| 869 | UGUGAGGUUGGCAUUGUUGUCU | hsa-miR-1294 |
| 870 | UCAAAACUGAGGGGCAUUUUCU | hsa-miR-1323 |
| 871 | GAUGAUGCUGCUGAUGCUG | hsa-miR-1322 |
| 872 | CUGGACUGAGCCGUGCUACUGG | hsa-miR-1269 |
| 873 | CAGGAUGUGGUCAAGUGUUGUU | hsa-miR-1265 |
| 874 | AAGUAGUUGGUUUGUAUGAGAUGGUU | hsa-miR-1244 |
| 875 | UUUAGAGACGGGGUCUUGCUCU | hsa-miR-1303 |
| 876 | AUAUAUGAUGACUUAGCUUUU | hsa-miR-1259 |
| 877 | UAAUUGCUUCCAUGUUU | hsa-miR-302F |
| 878 | UAGCAAAAACUGCAGUUACUUU | hsa-miR-548P |
| 879 | CAAGUCUUAUUUGAGCACCUGUU | hsa-miR-1264 |
| 880 | AGAGGAUACCCUUUGUAUGUU | hsa-miR-1185 |
| 881 | CGGAUGAGCAAAGAAAGUGGUU | hsa-miR-1255B |
| 882 | UAAGUGCUUCCAUGCUU | hsa-miR-302E |
| 883 | UCGUUUGCCUUUUUCUGCUU | hsa-miR-1282 |
| 884 | AGGAUGAGCAAAGAAAGUAGAUU | hsa-miR-1255A |
| 885 | CUGGAGAUAUGGAAGAGCUGUGU | hsa-miR-1270 |
| 886 | UAGGACACAUGGUCUACUUCU | hsa-miR-1197 |
| 887 | CAGGGAGGUGAAUGUGAU | hsa-miR-1321 |
| 888 | UGAGGCAGUAGAUUGAAU | hsa-miR-1827 |
| 889 | CCAGACAGAAUUCUAUGCACUUUC | hsa-miR-1324 |
| 890 | AAAAGUAAUCGCGGUUUUUGUC | hsa-miR-548H |
| 891 | AGCCUGGAAGCUGGAGCCUGCAGU | hsa-miR-1254 |
| 892 | AAAAGUACUUGCGGAUUUUGCU | hsa-miR-548K |
| 893 | ACUCUAGCUGCCAAAGGCGCU | hsa-miR-1251 |
| 894 | UCUGGGCAACAAAGUGAGACCU | hsa-miR-1285 |
| 895 | AAGUGAUCUAAAGGCCUACAU | hsa-miR-1245 |
| 896 | UGGGAACGGGUUCCGGCAGACGCUG | hsa-miR-1292 |
| 897 | UCAGCUGGCCCUCAUUUC | hsa-miR-1207-3P |
| 898 | UUGCAGCUGCCUGGGAGUGACUUC | hsa-miR-1301 |
| 899 | UGCUGGAUCAGUGGUUCGAGUC | hsa-miR-1287 |
| 900 | CUCCUGAGCCAUUCUGAGCCUC | hsa-miR-1200 |
| 901 | GAGGGUCUUGGGAGGGAUGUGAC | hsa-miR-1182 |
| 902 | UGGACUGCCCUGAUCUGGAGA | hsa-miR-1288 |
| 903 | UCCCACCGCUGCCACCC | hsa-miR-1280 |
| 904 | UGGCCCUGACUGAAGACCAGCAGU | hsa-miR-1291 |
| 905 | GUGGGGGAGAGGCUGUC | hsa-miR-1275 |
| 906 | CACUGUAGGUGAUGGUGAGAGUGGGCA | hsa-miR-1183 |
| 907 | CCUGCAGCGACUUGAUGGCUUCC | hsa-miR-1184 |
| 908 | UAAAGAGCCCUGUGGAGACA | hsa-miR-1276 |
| 909 | AAAAGCUGGGUUGAGAGGGCAA | hsa-miR-320B |
| 910 | GAUGAUGAUGGCAGCAAAUUCUGAAA | hsa-miR-1272 |
| 911 | UUUCCGGCUCGCGUGGGUGUGU | hsa-miR-1180 |
| 912 | AGGCAUUGACUUCUCACUAGCU | hsa-miR-1256 |
| 913 | UAGUACUGUGCAUAUCAUCUAU | hsa-miR-1278 |
| 914 | AUGGGUGAAUUUGUAGAAGGAU | hsa-miR-1262 |
| 915 | AACUGGAUCAAUUAUAGGAGUG | hsa-miR-1243 |
| 916 | GGUGGCCCGGCCGUGCCUGAGG | hsa-miR-663B |
| 917 | GUGCCAGCUGCAGUGGGGGAG | hsa-miR-1202 |
| 918 | AGAAGGAAAUUGAAUUCAUUUA | hsa-miR-1252 |
| 919 | UUCAUUCGGCUGUCCAGAUGUA | hsa-miR-1298 |
| 920 | UUAGGCCGCAGAUCUGGGUGA | hsa-miR-1295 |
| 921 | UGGAUUUUUGGAUCAGGGA | hsa-miR-1290 |
| 922 | UUUUCAACUCUAAUGGGAGAGA | hsa-miR-1305 |
| 923 | ACGCCCUUCCCCCCCUUCUUCA | hsa-miR-1249 |
| 924 | ACCUUCUUGUAUAAGCACUGUGCUAAA | hsa-miR-1248 |
| 925 | UGGAGUCCAGGAAUCUGCAUUUU | hsa-miR-1289 |
| 926 | UCGUGGCCUGGUCUCCAUUAU | hsa-miR-1204 |
| 927 | AUUGAUCAUCGACACUUCGAACGCAAU | hsa-miR-1826 |
| 928 | UUUGAGGCUACAGUGAGAUGUG | hsa-miR-1304 |
| 929 | GCAUGGGUGGUUCAGUGG | hsa-miR-1308 |
| 930 | CCCGGAGCCAGGAUGCAGCUC | hsa-miR-1203 |
| 931 | UGUUCAUGUAGAUGUUUAAGC | hsa-miR-1206 |
| 932 | AAAACUGUAAUUACUUUUGUAC | hsa-miR-548G |
| 933 | UCACUGUUCAGACAGGCGGA | hsa-miR-1208 |
| 934 | AAAAACUGAGACUACUUUUGCA | hsa-miR-548E |
| 935 | GUCCCUGUUCAGGCGCCA | hsa-miR-1274A |
| 936 | UCCCUGUUCGGGCGCCA | hsa-miR-1274B |
| 937 | CCUGUUGAAGUGUAAUCCCCA | hsa-miR-1267 |
| 938 | ACGGUGCUGGAUGUGGCCUUU | hsa-miR-1250 |
| 939 | CAAAAGUAAUUGUGGAUUUUGU | hsa-miR-548N |
| 940 | UCUACAAAGGAAAGCGCUUUCU | hsa-miR-1283 |
| 941 | ACCCGUCCCGUUCGUCCCCGGA | hsa-miR-1247 |
| 942 | AGAGAAGAAGAUCAGCCUGCA | hsa-miR-1253 |
| 943 | UCUCGCUGGGGCCUCCA | hsa-miR-720 |
| 944 | AUCCCACCUCUGCCACCA | hsa-miR-1260 |
| 945 | UAUUCAUUUAUCCCCAGCCUACA | hsa-miR-664 |
| 946 | UUGGGACAUACUUAUGCUAAA | hsa-miR-1302 |
| 947 | UUGAGAAGGAGGCUGCUG | hsa-miR-1300 |
| 243 | UCUAUACAGACCCUGGCUUUUC | hsa-miR-1284 |
| 244 | AAAAGUAUUUGCGGGUUUUGUC | hsa-miR-548L |
| 214 | UGGGUGGUCUGGAGAUUUGUGC | hsa-miR-1293 |
| 215 | UCCAGUGCCCUCCUCUCC | hsa-miR-1825 |
| 216 | UUAGGGCCCUGGCUCCAUCUCC | hsa-miR-1296 |
| 217 | AAAAGUAAUUGCGGAUUUUGCC | hsa-miR-548I |
| 102 | AGUGAAUGAUGGGUUCUGACC | hsa-miR-1257 |
| 99 | UCACACCUGCCUCGCCCCCC | hsa-miR-1228 |
| 13 | GACACGGGCGACAGCUGCGGCCC | hsa-miR-602 |
| 12 | CUUCCUCGUCUGUCUGCCCC | hsa-miR-1238 |

*denotes minor sequence as provided by the miRBase database, publicly available at (www.mirbase.org).
MiRNAs included in the miRNA signature are bolded.

### EXAMPLES

### Example 1

### Materials and Methods

### Cell Culture

The following breast cancer cell lines were obtained from the Harris Lab at Yale University School of Medicine: BT-474, SK-BR-3, MDA-MB-361 (MD361), MDA-MB-453 (MD453), UACC812, and UACC893 (labeled “parentals,” or untreated). A second stock of BT-474 cells was obtained from the Kute Lab at Wake Forest University (Yakes F M et al. (2002) Cancer Res 62: 4132-4141). These cell lines were maintained in RPMI 1640 with penicillin/streptomycin, 5% L-glutamine, and 10% FBS. Cells were incubated at 37° C. with 5% carbon dioxide. Two additional cell lines that were developed from resistant BT-474 clones were also obtained from the Kute Lab. After treatment with 10 ug/ml of Herceptin for two weeks, these clones were mechanically separated and replaced in media containing 10 ug/ml where they grew as well as the BT-474 cell line in the absence of Herceptin. Herceptin was obtained from the Harris Lab. Cells were kept frozen in liquid nitrogen, suspended in Recovery Media (Gibco).

### Dose-Response Studies

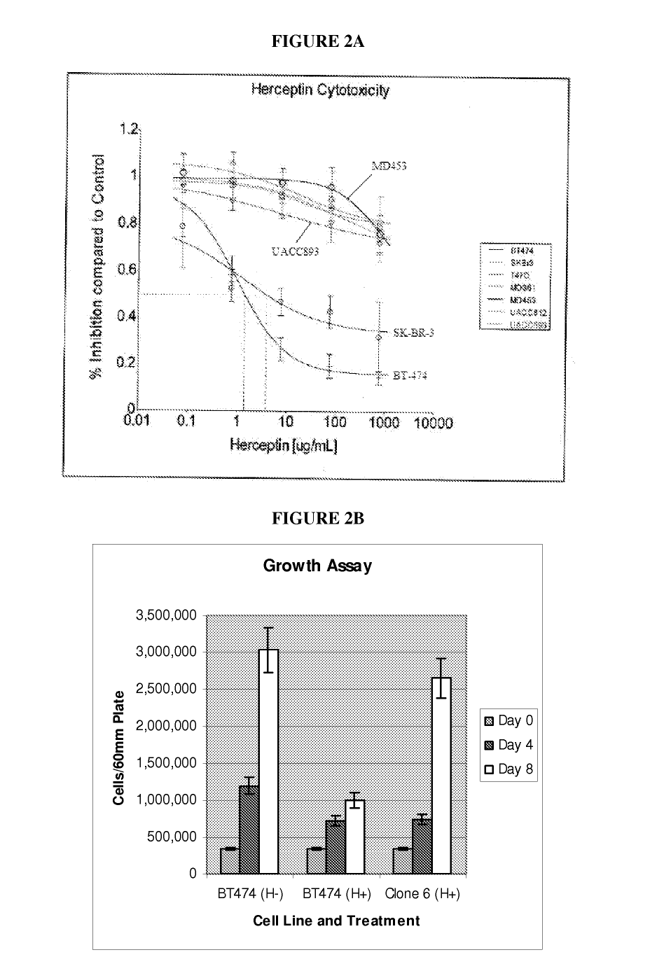

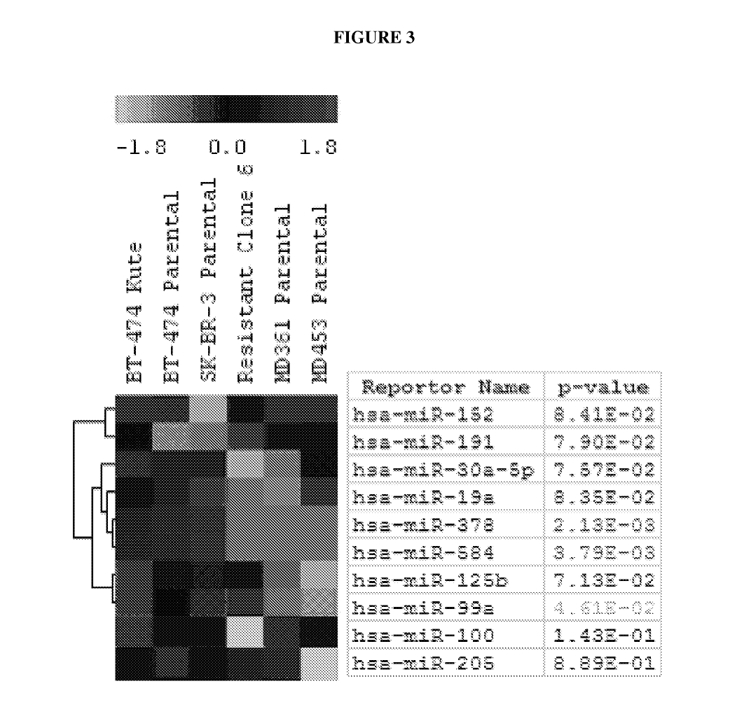

Cells were seeded at 5×10⁴ cells/well in 96-well dishes. After 24 hours, cells were treated in triplicate with serial dilutions of Herceptin in Opti-MEM at doses ranging from 0.1-750 μg/mL. After 5 days, CellTiter 96 Aqueous One solution was added to each well and cells were incubated for 1-2 hours, or until the untreated wells were brown. The plate was read with a SPECTRAax M2 plate reader. Growth inhibition was calculated by converting optical density values to percentages of viable cells compared with untreated cultures. To confirm growth assays on BT-474 and Resistant Clone 6 (previously performed by Dr. Kute), 300,000 cells were plated in 60 mm dishes. Six plates of BT-474 cells were left untreated, while six plates were treated with 10 μg/ml. Resistant Clone 6 cells were only treated. Three plates of each were counted on days 4 and 8 to produce FIG. 2B.

### Total RNA

Cells were harvested by trypsinizing 10 cm tissue culture dishes, and centrifuging to form a pellet from which media was discarded. Cells were washed with cold PBS in preparation for RNA collection. Total RNA was isolated immediately from 10⁷ cells using the mirVANA RNA Isolation Kit (Ambion, Inc., Austin, Tex.) according to the protocol of the manufacturer for total RNA isolation. Total RNA was quantified using the DU-64 spectrophotometer (Beckman) and stored at −80° C.

### MicroRNA Microarray and Statistical Analysis

A total of 10 μg was then subjected to microarray analysis. To confirm the quality of the RNA a UV test was performed and the samples were enriched for miRNAs by using a cut-off filter (μm100 from Microcon®—modified procedure). The microRNAs were then labeled and hybridized to a microarray chip with multiple repeat regions and a miRNA probe region, which detects miRNA transcripts listed in Sanger miRBase Release 9.0. This consists of 440 human miRNA sequences. Multiple control probes were included in each chip. The control probes were used for quality controls of chip production, sample labeling and assay conditions. For the in-depth data analysis of Herceptin-sensitive versus Herceptin-resistant samples, multi-array normalization and clustering analysis were performed. T-Tests were performed on two groups, sensitive and resistant, to identify miRNAs that were significantly differentiated between the sensitive and resistant cell lines.

### RT-qPCR

Reverse transcription of 10 ng of total RNA using specific Taqman miRNA probes (Applied Biosystems) yielded a cDNA template that was then amplified by quantitative PCR using Taqman Universal PCR Master Mix (Applied Biosystems). For normalization and relative quantitation, each sample was reverse transcribed and amplified with control primer RNU6B (CGCAAGGAUGACACGCAAAUUCGUGAAGCGUUCCAUAUUUUU, SEQ ID NO: 213). PCR conditions were 50° C. for 2 minutes and 95° C. for 10 minutes followed by 40 cycles of 95° C. for 15 seconds, 60° C. for 1 minute. miR-25, miR-99a, miR-100, miR-125b, miR-205, and let- 7a-specific probes were used.

### Transfection and Viability Assay.

Pre-miR-100 (5 nmol) was purchased from Ambion. This unprocessed oligo was used for transfection in combination with Xtreme Gene transfection agent. Cells were plated in 35 mm plates without penicillin/streptomycin. Twenty-four hours later the cells were transfected and allowed to sit for another 24 hours, at which point they were plated. For the viability assay, 5000 cells were plated per well in a 96-well plate. They were plated in media with varying concentrations of Herceptin (0-250 ug/ml). After 7 days, CellTiter was added (as mentioned in under cell culture) and results were analyzed.

### Example 2

### Herceptin Responsiveness in HER2 Positive Breast Cancer Cell Lines

For this study, several breast cancer cell lines that highly express HER-2 were obtained. HER2 expression levels in these breast cancer cell lines were analyzed by both IHC and FISH. The response of these HER2 positive breast cancer cells to Herceptin treatment was characterized (FIG. 2A), indicating that, as in tumor tissue, drug response spans a broad spectrum, from complete sensitivity (>50% growth inhibition after 5 days at concentrations above 10 ug/ml) to complete resistance (<5% growth inhibition observed after 5 days at concentrations up to 100 μg/ml). BT-474 and SK-BR-3 cell lines were sensitive while MD361 and MD453 were resistant to Herceptin. The incomplete resistance observed in UACC812 and UACC893 resulted in the exclusion of these cell lines from further analysis. In addition, two resistant clones expanded from BT-474 by treatment with Herceptin for two weeks were obtained. Both growth inhibition in BT-474 and lack of growth inhibition in Resistant Clone 6 by Herceptin (10 μg/ml) is shown in FIG. 2B, wherein cells were counted at three time points (0, 4, and 8 days).

### Example 3

### miRNA Profiling in Cell Lines Separates Lines by Herceptin Sensitivity

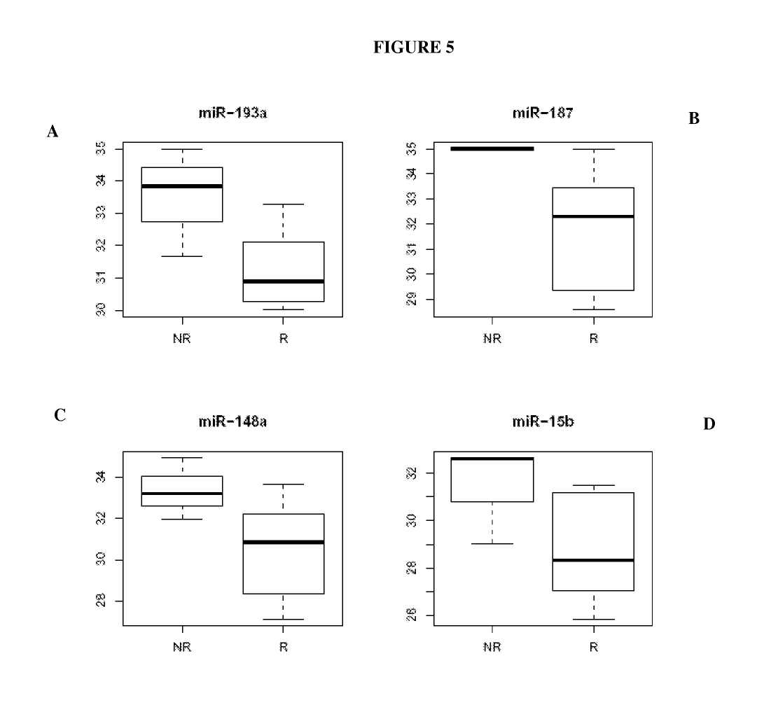

Total RNA was harvested from each cell line and microRNA microarray analysis was performed. Cluster analysis identified several miRNAs that differentiated the Herceptin sensitive from the Herceptin resistant cell lines. This included clustering the derived resistant BT474 clone with the Herceptin resistant cell lines. FIG. 4 shows miRNAs with significantly altered expression levels.

### Example 4

### miRNA Profiles in Human Her-2 Positive Tumors

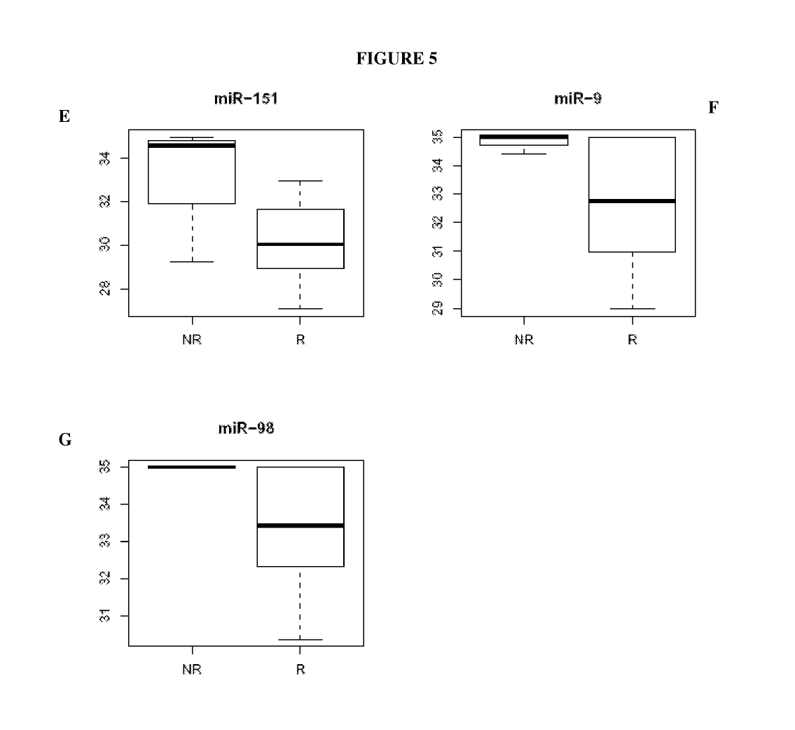

We and others have found that Her-2 positive tumors have unique miRNA signatures from other subtypes of breast cancer, especially the triple negative cohort. In addition however, we found that there was considerable heterogeneity within the Her-2 positive patient tumors (FIG. 5A-G).

### Example 5

### miRNA Signatures Separate Her-2 Positive Herceptin Responsive and Non-Responsive Tumors

To determine whether differences in miRNA signatures separate Herceptin responders from non-responders, miRNA signatures were compared in patients having tumors with known Herceptin responses. For the purposes of this study, patients having tumors with known Herceptin responses fell into one of two categories: those patients with metastatic disease and measured response or those patients who received Herceptin chemotherapy before surgery, with measured tumor responses. In the latter case only patients with pathologic complete responses (responders) or stable/progressive disease (non-responders) were used for the analysis.

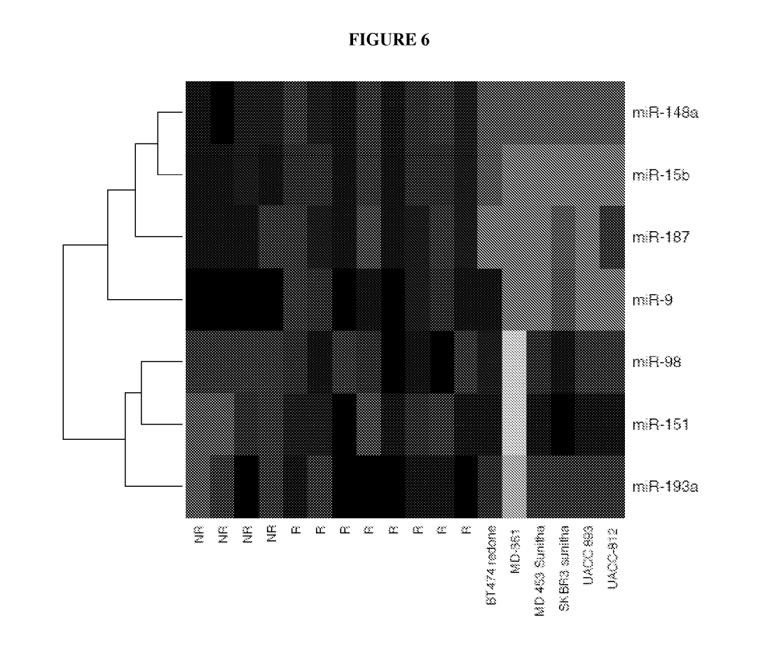

Using miRNA clustering methods of the invention, the analysis demonstrated that patients with Her-2 positive tumors that responded to Herceptin therapy had significantly different miRNA signatures than patients whose tumors did not respond to Herceptin therapy (FIGS. 4 and 5A-G). While in cell lines it was not determined that the same miRNAs predicted separation between Herceptin responsive lines and non-responsive lines, one of the cell lines (MD-361) did look most like the non-responders group, which was consistent with this cell line being non-responsive to Herceptin (FIG. 6).

Thus, a miRNA signature has been determined that differentiates between breast cancer patients having Her-2 positive tumors who do or do not respond to the anti-HER2 treatment, Herceptin.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

We claim:

1. A miRNA signature that indicates a HER2 positive breast cancer cell that is responsive to a HER2-targeted therapy, the signature comprising the determination of the decreased abundance of one or more miRNAs selected from the group consisting of hsa-miR-148a (SEQ ID NO: 92), hsa-miR-151 (SEQ ID NO: 205), hsa-miR-193a (SEQ ID NO: 114), hsa-miR-15b (SEQ ID NO: 27), hsa-miR-98 (SEQ ID NO: 191), hsa-miR-9 (SEQ ID NO: 196), hsa-miR-187 (SEQ ID NO: 109) compared to a HER2 positive breast cancer cell that is non-responsive to a HER2-targeted therapy, or the determination of the increased abundance of one or more miRNAs selected from the group consisting of hsa-miR-126 (SEQ ID NO: 76), hsa-miR-451 (SEQ ID NO: 271), and hsa-miR-218 (SEQ ID NO: 138) compared to a HER2 positive breast cancer cell that is non-responsive to a HER2-targeted therapy.

2. The miRNA signature of claim 1, wherein the HER2-targeted therapy is trastuzumab.

3. The miRNA signature of claim 1, wherein the HER2 positive breast cancer cell is positive for a second hormone receptor.

4. The miRNA signature of claim 1, wherein the second hormone receptor is the estrogen receptor or the progesterone receptor.

5. A method of determining a miRNA signature that distinguishes between a HER2-positive breast tumor that is responsive to HER2-targeted therapy and a HER2-positive breast tumor that is non-responsive to HER2-targeted therapy, comprising:
(a) obtaining a sample of HER2-positive breast cancer that is non-responsive to HER2-targeted therapy;
(b) determining the expression level of one or more miRNAs selected from the group consisting of hsa-miR-148a, hsa-miR-151, hsa-miR-193a, hsa-miR-15b, hsa-miR-98, hsa-miR-9, hsa-miR-187, hsa-miR-126, hsa-miR-451, and hsa-miR-218 from said non-responsive tumor; and
(c) comparing the expression level of the isolated miRNA in said non-responsive sample to a known expression level of the isolated miRNA in a HER2-positive breast tumor that is responsive to HER2-targeted therapy;
wherein the presence of a statistically-significant difference between the observed expression level of the isolated miRNA and the known expression level of said miRNA specifies a miRNA signature that distinguishes between a HER2-positive breast tumor that is responsive to HER2-targeted therapy and a HER2-positive breast tumor that is non-responsive to HER2-targeted therapy.

6. The method of claim 5, wherein the statistically-significant difference is a decrease in the expression level of hsa-miR-126, hsa-miR-451, or hsa-miR-218 in the non-responsive sample compared to the known level.

7. The method of claim 5, wherein the statistically-significant difference is an increase in the expression level of hsa-miR-148a, hsa-miR-151, hsa-miR-193a, hsa-miR-15b, hsa-miR-98, hsa-miR-9, or hsa-miR-187 in the non-responsive sample compared to the known level.

8. The method of claim 5, wherein the known level is calculated, retrieved from a database, or obtained experimentally.

9. The method of claim 5, wherein the HER2-targeted therapy is trastuzumab.

10. The method of claim 5, wherein the non-responsive breast tumor resides in the breast or at a second location in the body.

11. The method of claim 5, wherein the determining step further comprises normalizing the isolated miRNA expression level from the non-responsive sample to a control RNA.

12. The method of claim 11, further comprising:
(a) normalizing the isolated miRNA expression level from a HER2 positive breast tumor that is responsive to a HER2-targeted therapy to a control RNA; and
(b) comparing the expression levels of the isolated miRNA from the non-responsive and responsive samples,
wherein the presence of a statistically-significant difference between the expression levels of the isolated miRNA in the non-responsive and the responsive samples specifies a miRNA signature that distinguishes between a HER2-positive breast tumor that is responsive to HER2-targeted therapy and a HER2-positive breast tumor that is non-responsive to HER2-targeted therapy.

13. The method of claim 5, wherein the control RNA is a non-coding RNA selected from the group consisting of transfer RNA (tRNA), small nuclear RNA (snRNA) and small nucleolar RNA (snoRNA).

14. The method of claim 5, wherein the control RNA is a non-coding RNA of between 45 and 200 nucleotides.

15. The method of claim 5, wherein the control RNA is highly- and invariably-expressed between a responsive and non-responsive breast tumor.

16. A method of predicting the responsiveness of a breast tumor to HER-2-targeted therapy, comprising detecting the presence or absence of the miRNA signature of claim 1 in a sample from a breast tumor, wherein the presence of the miRNA signature within the sample indicates that the breast tumor is responsive to HER-2-targeted therapy.

17. The method of claim 16, wherein the HER-2-targeted therapy is trastuzumab.

18. The method of claim 16, wherein the breast tumor resides in the breast or at a second location in the body.

19. The method of claim 16, wherein the detecting step further comprises normalizing the miRNA expression level of the isolated miRNA to a control RNA.

20. The method of claim 16, wherein the control RNA is RNU6B (SEQ ID NO: 213).
